(19) United States
(12) Patent Application Publication
(10) Pub. No.: US 2022/0339143 A1
(43) Pub. Date: Oct. 27, 2022

(54) TAK-925 FOR USE IN TREATING NARCOLEPSY

(71) Applicant: Takeda Pharmaceutical Company Limited, Osaka (JP)

(72) Inventors: Deborah HARTMAN, Cambridge, MA (US); Rebecca EVANS, Cambridge, MA (US); Helene FAESSEL, Cambridge, MA (US); Shinichiro TANAKA, Cambridge, MA (US)

(21) Appl. No.: 17/642,331

(22) PCT Filed: Sep. 12, 2020
(86) PCT No.: PCT/IB2020/058483

§ 371 Date: Mar. 11, 2022

Related U.S. Application Data
(60) Provisional application No. 63/031,686, filed on May 29, 2020.
(60) Provisional application No. 62/900,298, filed on Sep. 13, 2019.

(51) Int. Cl. A61K 31/445; A61P 25/26; A61K 9/00

(52) U.S. Cl. CPC A61K 31/445; A61P 25/26; A61K 9/0019

(57) ABSTRACT

A method for treating narcolepsy type 1 in a subject in need thereof is disclosed, comprising administering to the subject an effective amount of methyl 3-((methylsulfonyl)anhino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. Compositions for treating narcolepsy type 1 comprising Compound (I) are also disclosed.

### CROSS-REFERENCE TO RELATED APPLICATIONS

This application claims priority to U.S. Provisional Application No. 62/900,298 filed Sep. 13, 2019 and 63/031,686 filed May 29, 2020.

### BACKGROUND OF THE INVENTION

Narcolepsy is a severe neurological disorder characterized by excessive daytime sleepiness (EDS) and cataplexy. Additional symptoms include hypnagogic/hypnopompic hallucinations, sleep paralysis and disturbed nighttime sleep, which altogether comprise the narcolepsy symptom pentad. Stimulants (e.g. modafinil) show certain level of effect for EDS and antidepressants (e.g. clomipramine) are used for cataplexy treatment. Sodium oxybate and pitolisant treat both EDS and cataplexy. However, the current therapies do not completely address the full extent and spectrum of narcolepsy symptoms in clinical practice.

Narcolepsy type 1 is associated with the loss of orexin producing neurons. The orexin (OX) receptor is a G-protein-coupled receptor that has 2 subtypes, orexin type-1 receptor (OX1R) and orexin type-2 receptor (OX2R). Upon activation, OX1R and OX2R couple with Gq protein to increase intracellular calcium (Ca²⁺) concentration. This application discloses methyl 3-((methylsulfonyl)amino)-2-(((4-phenylcyclohexyl)oxy)methyl)piperidine-1-carboxylate (Compound (I)), compositions comprising Compound (I), and the use of Compound (I) for the treatment of narcolepsy type 1 and/or one or more symptoms of narcolepsy type 1.

### SUMMARY OF THE INVENTION

Disclosed herein is a method for treating narcolepsy type 1 in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

In some embodiments, Cmax for administration of Compound (I) is about 8.30 ng/mL or more.

In some embodiments, AUC∞ for administration of Compound (I) is about 75.6 ng*h/mL or more.

In some embodiments, Cmax/Dose for administration of Compound (I) is about 1.66 ng/mL/mg or more.

In some embodiments, AUC∞/Dose for administration of Compound (I) is about 15.1 ng*h/mL/mg or more.

In some embodiments, the administration is non-oral administration. In some embodiments, the non-oral administration is intravenous administration, subcutaneous administration, transdermal administration or transmucosal administration. In some embodiments, the non-oral administration is intravenous administration.

In some embodiments, the administration is a single daily administration or a multiple daily administration.

Further disclosed herein is a method for increasing wakefulness or decreasing excessive sleepiness in a subject in need thereof, comprising administering to the subject an effective amount of methyl 3-((methylsulfonyl)amino)-2-(((4-phenylcyclohexyl)oxy)methyl) piperidine-1-carboxylate (Compound (I)), or a salt thereof, wherein plasma concentration for Compound (I) is about 5.04 ng/mL or more for about 1 hour or more. In some embodiments, the administration is non-oral administration.

Further disclosed herein is a method for decreasing cataplexy-like events in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, the administration is non-oral administration.

Further disclosed herein is a method for increasing intracellular calcium concentration in a subject in need thereof, comprising administering to the subject methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

Further disclosed herein is a method for increasing sleep latency in maintenance of wakefulness test (MWT) in a subject in need thereof, comprising administering to the subject an effective amount of methyl 3-((methylsulfonyl)amino)-2-(((4-phenylcyclohexyl)oxy) methyl)piperidine-1-carboxylate (Compound (I)), or a salt thereof, wherein plasma concentration for Compound (I) is about 5.04 ng/mL or more for about 1 hour or more. In some embodiments, the administration is non-oral administration.

Further disclosed herein is a method for improving Karolinska Sleepiness Scale (KSS) rating in a subject in need thereof, comprising administering to the subject an effective amount of methyl 3-((methylsulfonyl)amino)-2-(((4-phenylcyclohexyl)oxy)methyl) piperidine-1-carboxylate (Compound (I)), or a salt thereof, wherein plasma concentration for Compound (I) is about 5.04 ng/mL or more for about 1 hour or more. In some embodiments, the administration is non-oral administration.

Further disclosed herein is a method for treating a disease associated with reduced orexin level in a subject in need thereof, comprising administering to the subject an effective amount of methyl 3-((methylsulfonyl)amino)-2-(((4-phenylcyclohexyl)oxy)methyl) piperidine-1-carboxylate (Compound (I)), or a salt thereof, wherein plasma concentration for Compound (I) is about 5.04 ng/mL or more for about 1 hour or more. In some embodiments, the administration is non-oral administration.

In some embodiments, the effective amount is between about 3 mg to about 500 mg. In some embodiments, the effective amount is between about 5 mg to about 300 mg. In some embodiments, the effective amount is between about 5 mg to about 100 mg. In some embodiments, the effective amount is between about 5 mg to about 50 mg.

In some embodiments, Compound (I) is administered at least once per day.

In some embodiments, any of the methods disclosed herein further comprise administering one or more additional therapies. In some embodiments, the one or more additional therapies is selected from a stimulant, antidepressant, central nervous system depressant, and histamine 3 (H3) receptor antagonist.

In some embodiments of any of the methods disclosed herein, the plasma concentration for Compound (I) of about 5.04 ng/ml or more for a period of about 1 hour or more represents an average plasma concentration level for a group of treated subjects and the time period of 1 hour or more begins at any time point following administration.

In some embodiments of any of the methods disclosed herein, the plasma concentration for Compound (I) of about 5.04 ng/ml or more for a period of about 1 hour or more represents the plasma concentration level for the individually treated subject and the time period of 1 hour or more begins at any time point following administration to that subject.

In some embodiments, Compound (I) is an optically active compound. In some embodiments, Compound (I) is methyl (2R,3S)-3-((methylsulfonyl)amino)-2-(((cis-4-phenylcyclohexyl)oxy)methyl)piperidine-1-carboxylate (Compound (A)).

Further disclosed herein is a pharmaceutical composition comprising (a) methyl 3-((methylsulfonyl)amino)-2-(((4-phenylcyclohexyl)oxy)methyl)piperidine-1-carboxylate (Compound (I)), or a salt thereof; and (b) a pharmaceutically acceptable carrier therefor, which provides a plasma concentration for Compound (I) of about 5.04 ng/mL or more for about 1 hour or more.

In some embodiments, the pharmaceutical composition provides a Cmax for Compound (I) of about 8.30 ng/mL or more.

In some embodiments, the pharmaceutical composition provides an AUC∞ for Compound (I) of about 75.6 ng*h/mL or more.

In some embodiments, the pharmaceutical composition is formulated for non-oral administration.

In some embodiments, Compound (I) is an optically active compound. In some embodiments, Compound (I) is methyl (2R,3S)-3-((methylsulfonyl)amino)-2-(((cis-4-phenylcyclohexyl)oxy)methyl)piperidine-1-carboxylate (Compound (A)).

### BRIEF DESCRIPTION OF THE DRAWINGS

FIG. 1 shows the demographics and baseline characteristics of healthy subjects and healthy elderly subjects.

FIG. 2A-C show the demographics and baseline characteristics of subjects with narcolepsy type 1 (NT1 patients).

FIG. 3 shows treatment emergent adverse events (TEAEs) in healthy subjects from cohorts 1 and 2.

FIG. 4 shows treatment emergent adverse events (TEAEs) in healthy subjects from cohorts S1, S2, 3, and 4.

FIG. 5 shows treatment emergent adverse events (TEAEs) subjects with narcolepsy type 1 (NT1 patients).

FIG. 6 shows a mean and standard deviation plot of plasma concentrations of Compound A in healthy subjects from cohorts 1, 2, S1 and S2.

FIG. 7 shows the mean and standard deviation plot of plasma concentrations of Compound A after a single IV infusion of Compound A in patients with narcolepsy (pk analysis set). awk=awaking time, bed=bed time, SD=standard deviation.

FIG. 8 shows a bar chart of average sleep latency in MWT (Cohorts 5, 6, 7) (PD analysis set).

FIG. 9A shows the means plot of sleep latency in each session in MWT (Cohorts 5, 6, 7) (PD analysis set).

FIG. 9B shows the Least Square (LS) mean differences plot of sleep latency in each session in MWT (Cohorts 5, 6, 7) (PD analysis set).

FIG. 10A shows the means plot of daytime sleepiness as assessed by KSS by visit (Cohorts 5, 6, 7) (PD analysis set).

FIG. 10B shows the Least Square (LS) mean differences plot of daytime sleepiness as assessed by KSS by visit (Cohorts 5, 6, 7) (PD Analysis Set).

FIG. 11 shows an overview of the Study Schedule (NT1 Patients).

FIG. 12 shows an overview of Schedule of Study Procedures (NT1 Patients).

FIG. 13A shows mean and standard deviation plot of plasma concentrations of Compound A given as a 9-hour IV infusion on day 1 in NT1 patients (Cohorts B1, B2) (PK Set).

FIG. 13B shows mean and standard deviation plot of plasma concentrations of Compound A given as a 9-hour IV infusion on day 7 in NT1 patients (Cohorts B1, B2) (PK Set).

FIG. 14A shows average sleep latency in MWT (Cohorts B1, B2).

FIG. 14B shows change from baseline by Visit (Cohorts B1, B2).

FIG. 15 shows mean and standard deviation plot of sleep latency in each session in MWT by visit (Cohorts B1, B2).

FIG. 16 shows mean and standard deviation plot of change from time-matched baseline in KSS (Cohorts B1, B2).

FIG. 17A shows mean and standard deviation plot of change from time-matched baseline in PVT (Cohorts B1, B2).

FIG. 17B shows mean and standard deviation plot of change from time-matched baseline in PVT (Cohorts B1, B2).

### DETAILED DESCRIPTION OF THE INVENTION

Disclosed herein is methyl 3-((methylsulfonyl)amino)-2-(((4-phenylcyclohexyl)oxy) methyl)piperidine-1-carboxylate (Compound (I)), or a salt thereof, compositions and kits comprising Compound (I), or a salt thereof, and methods of using Compound (I), or a salt thereof.

Disclosed herein are methods for treating narcolepsy type 1 in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

Further disclosed herein is methyl 3-((methylsulfonyl)amino)-2-(((4-phenylcyclohexyl)oxy)methyl)piperidine-1-carboxylate (Compound (I)), or a salt thereof for use in treating narcolepsy type 1 in a subject in need thereof. In some embodiments, the plasma concentration for Compound (I) is about 5.04 ng/mL or more for about 1 hour or more.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for treating narcolepsy type 1 in a subject in need thereof. In some embodiments, the plasma concentration for Compound (I) is about 5.04 ng/mL or more for about 1 hour or more.

Disclosed herein are methods for increasing wakefulness or decreasing excessive sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, orexin level in the subject is compromised or partially compromised. In some embodiments, orexin level in the subject is reduced, below normal or below average. In some embodiments, the orexin level of the subject in need thereof is cerebral spinal fluid (CSF) hypocretin 1 (orexin A) concentration, measured by immunoreactivity, that is either 110 pg/mL or less, or less than one-third of mean values obtained in normal subjects with the same standardized assay. In some embodiments, the subject is suffering from or diagnosed as narcolepsy (e.g., narcolepsy type 1). In some embodiments, the excessive sleepiness is daytime excessive sleepiness.

Further disclosed herein is methyl 3-((methylsulfonyl)amino)-2-(((4-phenylcyclohexyl)oxy)methyl)piperidine-1-carboxylate (Compound (I)), or a salt thereof for use in increasing wakefulness or decreasing excessive sleepiness in a subject in need thereof. In some embodiments, the plasma concentration for Compound (I) is about 5.04 ng/mL or more for about 1 hour or more. In some embodiments, orexin level in the subject is compromised or partially compromised. In some embodiments, orexin level in the subject is reduced, below normal or below average. In some embodiments, the orexin level of the subject in need thereof is cerebral spinal fluid (CSF) hypocretin 1 (orexin A) concentration, measured by immunoreactivity, that is either 110 pg/mL or less, or less than one-third of mean values obtained in normal subjects with the same standardized assay. In some embodiments, the subject is suffering from or diagnosed as narcolepsy (e.g., narcolepsy type 1). In some embodiments, the excessive sleepiness is daytime excessive sleepiness.

Further disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing wakefulness or decreasing excessive sleepiness in a subject in need thereof. In some embodiments, the plasma concentration for Compound (I) is about 5.04 ng/mL or more for about 1 hour or more. In some embodiments, orexin level in the subject is compromised or partially compromised. In some embodiments, orexin level in the subject is reduced, below normal or below average. In some embodiments, the orexin level of the subject in need thereof is cerebral spinal fluid (CSF) hypocretin 1 (orexin A) concentration, measured by immunoreactivity, that is either 110 pg/mL or less, or less than one-third of mean values obtained in normal subjects with the same standardized assay. In some embodiments, the subject is suffering from or diagnosed as narcolepsy (e.g., narcolepsy type 1). In some embodiments, the excessive sleepiness is daytime excessive sleepiness.

Alternatively, the method for decreasing a cataplexy-like event (e.g., cataplexy) in a subject in need thereof, comprises administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1. In some embodiments, the cataplexy-like event is cataplexy.

Further disclosed herein is methyl 3-((methylsulfonyl)amino)-2-(((4-phenylcyclohexyl)oxy)methyl)piperidine-1-carboxylate (Compound (I)), or a salt thereof for use in decreasing a cataplexy-like event (e.g., cataplexy) in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1. In some embodiments, the cataplexy-like event is cataplexy.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for decreasing a cataplexy-like event (e.g., cataplexy) in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1. In some embodiments, the cataplexy-like event is cataplexy.

Further disclosed herein are methods for increasing intracellular calcium concentration in a subject in need thereof, comprising administering to the subject methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Further disclosed herein is methyl 3-((methylsulfonyl)amino)-2-(((4-phenylcyclohexyl)oxy)methyl)piperidine-1-carboxylate (Compound (I)), or a salt thereof for use in increasing intracellular calcium concentration in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing intracellular calcium concentration in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Disclosed herein are methods for increasing sleep latency in maintenance of wakefulness test (MWT) in a subject in need thereof, comprising administering to the subject an effective amount of methyl 3-((methylsulfonyl)amino)-2-(((4-phenylcyclohexyl) oxy)methyl)piperidine-1-carboxylate (Compound (I)), or a salt thereof, wherein plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Further disclosed herein is methyl 3-((methylsulfonyl)amino)-2-(((4-phenylcyclohexyl)oxy)methyl)piperidine-1-carboxylate (Compound (I)), or a salt thereof for use in increasing sleep latency in maintenance of wakefulness test (MWT) in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing sleep latency in maintenance of wakefulness test (MWT) in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Disclosed herein are methods for improving Karolinska Sleepiness Scale (KSS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Further disclosed herein is methyl 3-((methylsulfonyl)amino)-2-(((4-phenylcyclohexyl)oxy)methyl)piperidine-1-carboxylate (Compound (I)), or a salt thereof for use in improving Karolinska Sleepiness Scale (KS S) rating in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for improving Karolinska Sleepiness Scale (KSS) rating in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1.

Disclosed herein are methods for increasing wakefulness or decreasing excessive sleepiness for about 4 hours or more in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is maintained at about 5.04 ng/mL or more. In some embodiments, orexin level in the subject is compromised or partially compromised. In some embodiments, the subject is suffering from or diagnosed as narcolepsy type 1. In some embodiments, the excessive sleepiness is daytime excessive sleepiness.

Disclosed herein is methyl 3-((methylsulfonyl)amino)-2-(((4-phenylcyclohexyl) oxy)methyl)piperidine-1-carboxylate (Compound (I)), or a salt thereof for use in increasing wakefulness or decreasing excessive sleepiness for about 4 hours or more in a subject in need thereof. In some embodiments, the plasma concentration for Compound (I) is maintained at about 5.04 ng/mL or more. In some embodiments, orexin level in the subject is compromised or partially compromised. In some embodiments, the subject is suffering from or diagnosed as narcolepsy type 1. In some embodiments, the excessive sleepiness is daytime excessive sleepiness.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing wakefulness or decreasing excessive sleepiness for about 4 hours or more in a subject in need thereof. In some embodiments, the plasma concentration for Compound (I) is maintained at about 5.04 ng/mL or more. In some embodiments, orexin level in the subject is compromised or partially compromised. In some embodiments, the subject is suffering from or diagnosed as narcolepsy type 1. In some embodiments, the excessive sleepiness is daytime excessive sleepiness.

Disclosed herein are methods for decreasing or treating excessive sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1. In some embodiments, the excessive sleepiness is daytime excessive sleepiness.

Disclosed herein is methyl 3-((methylsulfonyl)amino)-2-(((4-phenylcyclohexyl) oxy)methyl)piperidine-1-carboxylate (Compound (I)), or a salt thereof for use in decreasing or treating excessive sleepiness in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1. In some embodiments, the excessive sleepiness is daytime excessive sleepiness.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for decreasing or treating excessive sleepiness in a subject in need thereof. In some embodiments, the plasma concentration for Compound (I) is about 5.04 ng/mL or more for about 1 hour or more. In some embodiments, orexin level in the subject is compromised or partially compromised. In some embodiments, the subject is suffering from or diagnosed as narcolepsy type 1. In some embodiments, the excessive sleepiness is daytime excessive sleepiness.

Disclosed herein are methods for treating a disease associated with reduced orexin level in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

Further disclosed herein is methyl 3-((methylsulfonyl)amino)-2-(((4-phenylcyclohexyl)oxy)methyl)piperidine-1-carboxylate (Compound (I)), or a salt thereof for use in treating a disease associated with reduced orexin level in a subject in need thereof. In some embodiments, the plasma concentration for Compound (I) is about 5.04 ng/mL or more for about 1 hour or more.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for treating a disease associated with reduced orexin level in a subject in need thereof. In some embodiments, the plasma concentration for Compound (I) is about 5.04 ng/mL or more for about 1 hour or more.

Disclosed herein are methods for increasing alertness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof. In some embodiments, the plasma concentration for Compound (I) is about 5.04 ng/mL or more for about 1 hour or more. In some embodiments, the plasma concentration for Compound (I) is about 10.48 ng/mL or more for about 1 hour or more. In some embodiments, the subject is suffering from or diagnosed as narcolepsy type 1.

Disclosed herein is methyl 3-((methylsulfonyl)amino)-2-(((4-phenylcyclohexyl)oxy)methyl)piperidine-1-carboxylate (Compound (I)), or a salt thereof for use in increasing alertness in a subject in need thereof. In some embodiments, the plasma concentration for Compound (I) is about 5.04 ng/mL or more for about 1 hour or more. In some embodiments, the plasma concentration for Compound (I) is about 10.48 ng/mL or more for about 1 hour or more. In some embodiments, the subject is suffering from or diagnosed as narcolepsy type 1.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing alertness in a subject in need thereof. In some embodiments, the plasma concentration for Compound (I) is about 5.04 ng/mL or more for about 1 hour or more. In some embodiments, the plasma concentration for Compound (I) is about 10.48 ng/mL or more for about 1 hour or more. In some embodiments, the subject is suffering from or diagnosed as narcolepsy type 1.

Disclosed herein are methods for improving Epworth Sleepiness Scale (ESS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more. In some embodiments, the plasma concentration for Compound (I) is about 17.65 ng/mL or more for about 1 hour or more. In some embodiments, the subject is suffering from or diagnosed as narcolepsy type 1.

Further disclosed herein is methyl 3-((methylsulfonyl)amino)-2-(((4-phenylcyclohexyl)oxy)methyl)piperidine-1-carboxylate (Compound (I)), or a salt thereof for use in improving Epworth Sleepiness Scale (ESS) rating in a subject in need thereof. In some embodiments, the plasma concentration for Compound (I) is about 5.04 ng/mL or more for about 1 hour or more. In some embodiments, the plasma concentration for Compound (I) is about 17.65 ng/mL or more for about 1 hour or more. In some embodiments, the subject is suffering from or diagnosed as narcolepsy type 1.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for improving Epworth Sleepiness Scale (ESS) rating in a subject in need thereof. In some embodiments, the plasma concentration for Compound (I) is about 5.04 ng/mL or more for about 1 hour or more. In some embodiments, the plasma concentration for Compound (I) is about 17.65 ng/mL or more for about 1 hour or more. In some embodiments, the subject is suffering from or diagnosed as narcolepsy type 1.

The methods and uses disclosed herein may treat narcolepsy type 1 in a subject in need thereof. In some embodiments, treating narcolepsy type 1 may comprise reducing or alleviating one or more symptoms of narcolepsy type 1. The one or more symptoms of narcolepsy type 1 may be selected from excessive daytime sleepiness (EDS) and cataplexy. In some embodiments, the one or more symptoms of narcolepsy type 1 is selected from excessive daytime sleepiness (EDS) and cataplexy. Narcolepsy may be diagnosed by diagnostic criteria generally used in the field, e.g., the third edition of the International Classification of Sleep Disorders (ICSD-3) and the fifth edition of the Diagnostic and Statistical Manual of Mental Disorders (DSM-5).

The methods and uses disclosed herein may increase wakefulness and/or decrease and/or treat excessive sleepiness in a subject in need thereof. In some embodiments, excessive sleepiness as used herein is also known as excessive daytime sleepiness (EDS) or excessive need for sleep (ENS). In some embodiments, wakefulness and/or decrease and/or treatment of excessive sleepiness is determined by electroencephalogram (EEG) and/or electromyogram (EMG). In some embodiments, wakefulness and/or decrease of sleepiness is determined by using the Maintenance Wakefulness Test (MWT). The MWT may be quantified by EEG. An electroencephalogram (EEG) is a test that detects electrical activity in the brain using small, metal discs or electrodes attached to the scalp. In some embodiments, wakefulness and/or decrease of sleepiness is determined by using the multiple sleep latency test (MSLT) or the Oxford Sleep Resistance (OSLER) test. In some embodiments, the test is the Karolinska Sleepiness Scale (KSS), the Epworth Sleepiness Scale (ESS) or the Stanford Sleepiness Scale. In some embodiments, the subject with the excessive sleepiness may suffer from or be diagnosed with narcolepsy type 1, narcolepsy type 2, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); or disturbance of consciousness such as coma and the like; or narcolepsy syndrome accompanied by narcolepsy-like symptoms; hypersomnolence or hypersomnia syndrome accompanied by daytime hypersomnia (e.g., Parkinson's disease, Guillain-barre syndrome and Kleine Levin syndrome); Parkinson's disease, Alzheimer's Disease, DLB (Lewy body dementia), Prader-Willi Syndrome, depressions (depression, atypical depression, major depressive disorder, treatment resistant depression), ADHD or other disorders of vigilance; or residual excessive daytime sleepiness in sleep apnea syndrome (e.g., obstructive sleep apnea, obstructive sleep apnea with use of continuous positive airway pressure); or the like. In some embodiments, the excessive sleepiness of the subject may be caused by or associated with narcolepsy type 1, narcolepsy type 2, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); or disturbance of consciousness such as coma and the like; or narcolepsy syndrome accompanied by narcolepsy-like symptoms; hypersomnolence or hypersomnia syndrome accompanied by daytime hypersomnia (e.g., Parkinson's disease, Guillain-barre syndrome and Kleine Levin syndrome); Parkinson's disease, Alzheimer's Disease, DLB (Lewy body dementia), Prader-Willi Syndrome, depressions (depression, atypical depression, major depressive disorder, treatment resistant depression), ADHD or other disorders of vigilance; or residual excessive daytime sleepiness in sleep apnea syndrome (e.g., obstructive sleep apnea, obstructive sleep apnea with use of continuous positive airway pressure); or the like.

The methods and uses disclosed herein may decrease cataplexy-like events (e.g., cataplexy) in a subject in need thereof. In some embodiments, the number of cataplexy-like events is decreased by 1, 2, 3, 4, 5, 6, 7, 8, 9, 10, 11, 12, 13, 14, 15, 16, 17, 18, 19, or 20 or more cataplexy-like events. In some embodiments, the number of cataplexy-like events is decreased by at least 1, 2, 3, 4, 5, 6, 7, or 8 or more events in a 24 hour period. In some embodiments, the number of cataplexy-like events is decreased by 50%, 55%, 60%, 65%, 70%, 75%, 80%, 85%, 90% or 95% or more cataplexy-like events, comparing with the case a subject is not administered by Compound (I). In some embodiments, the number of cataplexy-like events is decreased by at least 1, 2, 3, 4, 5, 6, 7, 8, 9, 10, 11, 12, 13, 14, 15, 16, 17, 18, 19, or 20 or more events in a 1-, 2-, 3-, 4-, 5-, 6-, 7-, 8-, 9-, 10-, 11-, 12-, 13-, 14-, or 15-day period. The cataplexy-like events include cataplexy.

The methods and uses disclosed herein may increase sleep latency in maintenance of wakefulness test (MWT) in a subject in need thereof. In some embodiments, the sleep latency in MWT increased by at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% or more. In some embodiments, the sleep latency in MWT increased by at least 1, 2, 3, 4, 5, 6, 7, 8, 9, 10, 11, 12, 13, 14, 15, 16, 17, 18, 19, or 20 or more minutes.

The methods and uses disclosed herein may decrease daytime sleepiness or improve Karolinska Sleepiness Scale (KSS) rating in a subject in need thereof. In some embodiments, the KSS rating is improved 1, 2, 3, 4, or 5 or more ratings. In some embodiments, the subject has a KSS rating of 1, 2, 3, 4, or 5 after treatment with Compound (I).

The methods and uses disclosed herein may increase intracellular calcium concentration in a subject in need thereof. In some embodiments, the intracellular calcium concentration is increased by at least 1%, 2%, 3%, 4%, 5%, 6%, 7%, 8%, 9%10%, 11%, 12%, 13%, 14%, 15%, 16%, 17%, 18%, 19%, or 20% or more.

The methods and uses disclosed herein may treat a disease associated with reduced orexin level in a subject in need thereof. The reduced orexin level in the subject means the orexin level of the subject is reduced, below normal or below average level of orexin of a subject who is not compromised or whose orexin level is normal. In some embodiments, the reduced orexin level of the subject is the cerebral spinal fluid (CSF) hypocretin 1 (orexin A) concentration, measured by immunoreactivity, that is either 110 pg/mL or less, or less than one-third of mean values obtained in normal subjects with the same standardized assay. In some embodiments, a disease associated with reduced orexin level may be narcolepsy type 1, narcolepsy type 2, hypersomnia, hypersomnolence, sleep apnea syndrome (e.g., obstructive sleep apnea, obstructive sleep apnea with use of continuous positive airway pressure); or disturbance of consciousness such as coma and the like; or narcolepsy syndrome accompanied by narcolepsy-like symptoms; hypersomnolence or hypersomnia syndrome accompanied by daytime hypersomnia (e.g., Parkinson's disease, Guillain-barre syndrome and Kleine Levin syndrome); excessive daytime sleepiness in Parkinson's disease, Alzheimer's Disease, DLB (Lewy body dementia), Prader-Willi Syndrome, depressions (depression, atypical depression, major depressive disorder, treatment resistant depression), ADHD, sleep apnea syndrome (e.g., obstructive sleep apnea, obstructive sleep apnea with use of continuous positive airway pressure) or other disorders of vigilance; or residual excessive daytime sleepiness in sleep apnea syndrome (e.g., obstructive sleep apnea, obstructive sleep apnea with use of continuous positive airway pressure); or the like.

The methods and uses disclosed herein may treat a disease selected from narcolepsy type 1, narcolepsy type 2, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); and disturbance of consciousness such as coma and the like; and narcolepsy syndrome accompanied by narcolepsy-like symptoms; hypersomnolence or hypersomnia syndrome accompanied by daytime hypersomnia (e.g., Parkinson's disease, Guillain-barre syndrome and Kleine Levin syndrome); excessive daytime sleepiness in Parkinson's disease, Alzheimer's Disease, DLB (Lewy body dementia), Prader-Willi Syndrome, depressions (depression, atypical depression, major depressive disorder, treatment resistant depression), ADHD, sleep apnea syndrome (e.g., obstructive sleep apnea, obstructive sleep apnea with use of continuous positive airway pressure) and other disorders of vigilance; or residual excessive daytime sleepiness in sleep apnea syndrome (e.g., obstructive sleep apnea, obstructive sleep apnea with use of continuous positive airway pressure); and the like.

The methods and uses disclosed herein may comprise performing one or more tests to quantify a subject's sleepiness. In some embodiments, the test is selected from the multiple sleep latency test (MSLT), maintenance of wakefulness test (MWT), and the Oxford Sleep Resistance (OSLER) test. In some embodiments, the test is MWT. In some embodiments, the test is the Karolinska Sleepiness Scale (KSS), the Epworth Sleepiness Scale (ESS) or the Stanford Sleepiness Scale.

The methods and uses disclosed herein comprise administering Compound (I) to a subject in need thereof. In some embodiments, Compound (I) is administered orally. In some embodiments, Compound (I) is administered non-orally. In some embodiments, the non-oral administration is intravenous administration, subcutaneous administration, transdermal administration, intradermal administration or transmucosal administration. In some embodiments, the non-oral administration is intravenous administration. In some embodiments, the non-oral administration is subcutaneous administration. In some embodiments, the non-oral administration is transdermal administration. In some embodiments, the non-oral administration is transmucosal administration. In some embodiments, Compound (I) is administered intravenously. Alternatively, or additionally, Compound (I) may be administered as an infusion. Administering Compound (I) as an infusion may comprise administering Compound (I) through a needle or catheter.

Compound (I) can be administered orally and non-orally such as intramuscular, intraperitoneal, intravenous, intraarterial, intraventricular, intracisternal injection or infusion; subcutaneous injection; or implant; or inhalation spray, intratracheal, nasal, vaginal, rectal, subdermal, transdermal, intradermal, epidural, ocular insert or ocular instillation administration, in a suitable unit dosage form containing a pharmaceutically acceptable conventional nontoxic carrier, adjuvant and vehicle suitable for each administration route.

In some embodiments, Compound (I) is administered as an infusion for at least 10, 20, 30, 40, 50, 60, 70, 80, 90, 100, 120, 140, 160, or 180 or more minutes. Compound (I) may be administered as an infusion for at least 1, 2, 3, 4, 5, 6, 7, 8, 9, 10, 11, 12, 13, 14, 15, 16, 17, 18, 19, or 20 or more hours. Compound (I) may be administered as an infusion for at least 2 hours. In some embodiments, the total time for administering Compound (I) is consecutive (e.g., OX2R is administered as an infusion for at least 2 consecutive hours). Alternatively, the total time for administering Compound (I) is intermittent (e.g., OX2R is administered as an infusion for 1 hour, then the infusion is stopped for period of time, and the infusion is restarted for another hour).

Alternatively, or additionally, administering Compound (I) may comprise administering an effective amount of Compound (I), in some embodiments, administering Compound (I) may comprise administering a therapeutically effective amount of Compound (I). The effective amount of Compound (I) may be between about 3 mg to about 500 mg. The effective amount of Compound (I) may be between about 5 mg to about 400 mg. The effective amount of Compound (I) may be between about 5 mg to about 300 mg of Compound (I). The effective amount of Compound (I) may be between about 5 mg to about 200 mg. The effective amount of Compound (I) may be between about 5 mg to 100 mg of Compound (I). The effective amount of Compound (I) may be between about 5 mg to 50 mg of Compound (I).

The effective amount of Compound (I) may be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115 or 120 mg. The effective amount of Compound (I) may be at least 3 mg. The effective amount of Compound (I) may be at least 4 mg. The effective amount of Compound (I) may be at least 5 mg. The effective amount of Compound (I) may be at least 6 mg. The effective amount of Compound (I) may be at least 7 mg. The effective amount of Compound (I) may be at least 10 mg. The effective amount of Compound (I) may be at least 15 mg. The effective amount of Compound (I) may be at least 20 mg. The effective amount of Compound (I) may be at least 30 mg. The effective amount of Compound (I) may be at least 40 mg. The effective amount of Compound (I) may be at least 50 mg.

The effective amount of Compound (I) may be less than 300, 290, 280, 275, 270, 260, 250, 240, 230, 225, 220, 210, 200, 175, 150, 125, 100, 90, 80, 70, 60, or 50 mg. The effective amount of Compound (I) may be less than 250 mg. The effective amount of Compound (I) may be less than 200 mg. The effective amount of Compound (I) may be less than 150 mg. The effective amount of Compound (I) may be less than 100 mg. The effective amount of Compound (I) may be less than 50 mg.

The effective amount of Compound (I) may be between 5 and 300 mg. The effective amount of Compound (I) may be between 5 and 250 mg. The effective amount of Compound (I) may be between 5 and 200 mg. The effective amount of Compound (I) may be between 5 and 150 mg. The effective amount of Compound (I) may be between 5 and 100 mg. The effective amount of Compound (I) may be between 5 and 50 mg.

The effective amount of Compound (I) may be between 7 and 300 mg. The effective amount of Compound (I) may be between 7 and 250 mg. The effective amount of Compound (I) may be between 7 and 200 mg. The effective amount of Compound (I) may be between 7 and 150 mg. The effective amount of Compound (I) may be between 7 and 100 mg. The effective amount of Compound (I) may be between 7 and 50 mg.

The effective amount of Compound (I) may be between 10 and 300 mg. The effective amount of Compound (I) may be between 10 and 250 mg. The effective amount of Compound (I) may be between 10 and 200 mg. The effective amount of Compound (I) may be between 10 and 150 mg. The effective amount of Compound (I) may be between 10 and 100 mg. The effective amount of Compound (I) may be between 10 and 50 mg.

Depending on the subject and the period for administration of Compound (I), the effective amount may change. In some embodiments, the effective amount of Compound (I) may be gradually increased during the administration period of Compound (I) for the purpose of performing the intended or desired effect, or achieving the same or desired plasma concentration for Compound (I). In some embodiments, the effective amount of Compound (I) is gradually increased within the range from about 5 mg to about 300 mg of Compound (I). In some embodiments, the effective amount of Compound (I) is gradually increased within the range from about 7 mg to about 300 mg of Compound (I). In some embodiments, the effective amount of Compound (I) is gradually increased within the range from about 10 mg to about 300 mg of Compound (I). In some embodiments, the effective amount of Compound (I) is gradually increased within the range from about 5 mg to about 200 mg of Compound (I). In some embodiments, the effective amount of Compound (I) is gradually increased within the range from about 7 mg to about 200 mg of Compound (I). In some embodiments, the effective amount of Compound (I) is gradually increased within the range from about 10 mg to 200 mg of Compound (I). In some embodiments, the effective amount of Compound (I) is gradually increased within the range from about 5 mg to about 100 mg of Compound (I). In some embodiments, the effective amount of Compound (I) is gradually increased within the range from about 7 mg to about 100 mg of Compound (I). In some embodiments, the effective amount of Compound (I) is gradually increased within the range from about 10 mg to 100 mg of Compound (I). In some embodiments, the effective amount of Compound (I) is gradually increased within the range from about 5 mg to about 50 mg of Compound (I). In some embodiments, the effective amount of Compound (I) is gradually increased within the range from about 7 mg to about 50 mg of Compound (I). In some embodiments, the effective amount of Compound (I) is gradually increased within the range from about 10 mg to about 50 mg of Compound (I).

The plasma concentration for Compound (I) may be about 5.04 ng/mL or more for about 1 hour or more.

The plasma concentration for Compound (I) may be at least 5.04, 6.38, 7.68, 7.72, 7.78, 8.75, 9.82, 10.09, 10.48, 12.40, 13.11, 14.38, 15.52, 15.65, 17.30, 17.65, 18.87, 19.08, 21.50, 24.13, 42.61, 57.64, 61.53, 73.30, 74.08, 80.45, 90.53, 96.00 or 118.70 ng/mL for at least 1 hour. The plasma concentration for Compound (I) may be at least 5.04, 6.38, 7.68, 7.72, 7.78, 8.75, 9.82, 10.09, 10.48, 12.40, 13.11, 14.38, 15.52, 15.65, 17.30, 17.65, 18.87, 19.08, 21.50, 24.13, 42.61, 57.64, 61.53, 73.30, 74.08, 80.45, 90.53, 96.00 or 118.70 ng/mL for at least 2 hours. The plasma concentration for Compound (I) may be at least 5.04, 6.38, 7.68, 7.72, 7.78, 8.75, 9.82, 10.09, 10.48, 12.40, 13.11, 14.38, 15.52, 15.65, 17.30, 17.65, 18.87, 19.08, 21.50, 24.13, 42.61, 57.64, 61.53, 73.30, 74.08, 80.45, 90.53, 96.00 or 118.70 ng/mL for at least 4 hours. The plasma concentration for Compound (I) may be at least 5.04, 6.38, 7.68, 7.72, 7.78, 8.75, 9.82, 10.09, 10.48, 12.40, 13.11, 14.38, 15.52, 15.65, 17.30, 17.65, 18.87, 19.08, 21.50, 24.13, 42.61, 57.64, 61.53, 73.30, 74.08, 80.45, 90.53, 96.00 or 118.70 ng/mL for at least 6 hours. The plasma concentration for Compound (I) may be at least 5.04, 6.38, 7.68, 7.72, 7.78, 8.75, 9.82, 10.09, 10.48, 12.40, 13.11, 14.38, 15.52, 15.65, 17.30, 17.65, 18.87, 19.08, 21.50, 24.13, 42.61, 57.64, 61.53, 73.30, 74.08, 80.45, 90.53, 96.00 or 118.70 ng/mL for at least 8 hours. The plasma concentration for Compound (I) may be at least 5.04, 6.38, 7.68, 7.72, 7.78, 8.75, 9.82, 10.09, 10.48, 12.40, 13.11, 14.38, 15.52, 15.65, 17.30, 17.65, 18.87, 19.08, 21.50, 24.13, 42.61, 57.64, 61.53, 73.30, 74.08, 80.45, 90.53, 96.00 or 118.70 ng/mL for at least 12 hours.

The plasma concentration for Compound (I) may be at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145 or 150 ng/mL for at least 1 hour. The plasma concentration for Compound (I) may be at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145 or 150 ng/mL for at least 2 hours. The plasma concentration for Compound (I) may be at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145 or 150 ng/mL for at least 4 hours. The plasma concentration for Compound (I) may be at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145 or 150 ng/mL for at least 6 hours. The plasma concentration for Compound (I) may be at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145 or 150 ng/mL for at least 8 hours. The plasma concentration for Compound (I) may be at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145 or 150 ng/mL for at least 12 hours.

The plasma concentration for Compound (I) may be between 5.04 and 500 ng/mL for at least 1, 2, 4, 6, 8 or 12 hours. The plasma concentration for Compound (I) may be between 5.04 and 400 ng/mL for at least 1, 2, 4, 6, 8 or 12 hours. The plasma concentration for Compound (I) may be between 5.04 and 300 ng/mL for at least 1, 2, 4, 6, 8 or 12 hours. The plasma concentration for Compound (I) may be between 5.04 and 200 ng/mL for at least 1, 2, 4, 6, 8 or 12 hours.

The plasma concentration for Compound (I) may be between 6.38 and 500 ng/mL for at least 1, 2, 4, 6, 8 or 12 hours. The plasma concentration for Compound (I) may be between 6.38 and 400 ng/mL for at least 1, 2, 4, 6, 8 or 12 hours. The plasma concentration for Compound (I) may be between 6.38 and 300 ng/mL for at least 1, 2, 4, 6, 8 or 12 hours. The plasma concentration for Compound (I) may be between 6.38 and 200 ng/mL for at least 1, 2, 4, 6, 8 or 12 hours.

The plasma concentration for Compound (I) may be between 7.72 and 500 ng/mL for at least 1, 2, 4, 6, 8 or 12 hours. The plasma concentration for Compound (I) may be between 7.72 and 400 ng/mL for at least 1, 2, 4, 6, 8 or 12 hours. The plasma concentration for Compound (I) may be between 7.72 and 300 ng/mL for at least 1, 2, 4, 6, 8 or 12 hours. The plasma concentration for Compound (I) may be between 7.72 and 200 ng/mL for at least 1, 2, 4, 6, 8 or 12 hours.

The plasma concentration for Compound (I) may be between 8.75 and 500 ng/mL for at least 1, 2, 4, 6, 8 or 12 hours. The plasma concentration for Compound (I) may be between 8.75 and 400 ng/mL for at least 1, 2, 4, 6, 8 or 12 hours. The plasma concentration for Compound (I) may be between 8.75 and 300 ng/mL for at least 1, 2, 4, 6, 8 or 12 hours. The plasma concentration for Compound (I) may be between 8.75 and 200 ng/mL for at least 1, 2, 4, 6, 8 or 12 hours.

The plasma concentration for Compound (I) may be between 10.09 and 500 ng/mL for at least 1, 2, 4, 6, 8 or 12 hours. The plasma concentration for Compound (I) may be between 10.09 and 400 ng/mL for at least 1, 2, 4, 6, 8 or 12 hours. The plasma concentration for Compound (I) may be between 10.09 and 300 ng/mL for at least 1, 2, 4, 6, 8 or 12 hours. The plasma concentration for Compound (I) may be between 10.09 and 200 ng/mL for at least 1, 2, 4, 6, 8 or 12 hours.

The plasma concentration for Compound (I) may be between 10.48 and 500 ng/mL for at least 1, 2, 4, 6, 8 or 12 hours. The plasma concentration for Compound (I) may be between 10.48 and 400 ng/mL for at least 1, 2, 4, 6, 8 or 12 hours. The plasma concentration for Compound (I) may be between 10.48 and 300 ng/mL for at least 1, 2, 4, 6, 8 or 12 hours. The plasma concentration for Compound (I) may be between 10.48 and 200 ng/mL for at least 1, 2, 4, 6, 8 or 12 hours.

The plasma concentration for Compound (I) may be between 14.38 and 500 ng/mL for at least 1, 2, 4, 6, 8 or 12 hours. The plasma concentration for Compound (I) may be between 14.38 and 400 ng/mL for at least 1, 2, 4, 6, 8 or 12 hours. The plasma concentration for Compound (I) may be between 14.38 and 300 ng/mL for at least 1, 2, 4, 6, 8 or 12 hours. The plasma concentration for Compound (I) may be between 14.38 and 200 ng/mL for at least 1, 2, 4, 6, 8 or 12 hours.

The plasma concentration for Compound (I) may be between 17.3 and 500 ng/mL for at least 1, 2, 4, 6, 8 or 12 hours. The plasma concentration for Compound (I) may be between 17.3 and 400 ng/mL for at least 1, 2, 4, 6, 8 or 12 hours. The plasma concentration for Compound (I) may be between 17.3 and 300 ng/mL for at least 1, 2, 4, 6, 8 or 12 hours. The plasma concentration for Compound (I) may be between 17.3 and 200 ng/mL for at least 1, 2, 4, 6, 8 or 12 hours.

The plasma concentration for Compound (I) may be between 17.65 and 500 ng/mL for at least 1, 2, 4, 6, 8 or 12 hours. The plasma concentration for Compound (I) may be between 17.65 and 400 ng/mL for at least 1, 2, 4, 6, 8 or 12 hours. The plasma concentration for Compound (I) may be between 17.65 and 300 ng/mL for at least 1, 2, 4, 6, 8 or 12 hours. The plasma concentration for Compound (I) may be between 17.65 and 200 ng/mL for at least 1, 2, 4, 6, 8 or 12 hours.

The plasma concentration for Compound (I) may be between 21.5 and 500 ng/mL for at least 1, 2, 4, 6, 8 or 12 hours. The plasma concentration for Compound (I) may be between 21.5 and 400 ng/mL for at least 1, 2, 4, 6, 8 or 12 hours. The plasma concentration for Compound (I) may be between 21.5 and 300 ng/mL for at least 1, 2, 4, 6, 8 or 12 hours. The plasma concentration for Compound (I) may be between 21.5 and 200 ng/mL for at least 1, 2, 4, 6, 8 or 12 hours.

The plasma concentration for Compound (I) may be between 61.53 and 500 ng/mL for at least 1, 2, 4, 6, 8 or 12 hours. The plasma concentration for Compound (I) may be between 61.53 and 400 ng/mL for at least 1, 2, 4, 6, 8 or 12 hours. The plasma concentration for Compound (I) may be between 61.53 and 300 ng/mL for at least 1, 2, 4, 6, 8 or 12 hours. The plasma concentration for Compound (I) may be between 61.53 and 200 ng/mL for at least 1, 2, 4, 6, 8 or 12 hours.

The plasma concentration for Compound (I) may be between 74.08 and 500 ng/mL for at least 1, 2, 4, 6, 8 or 12 hours. The plasma concentration for Compound (I) may be between 74.08 and 400 ng/mL for at least 1, 2, 4, 6, 8 or 12 hours. The plasma concentration for Compound (I) may be between 74.08 and 300 ng/mL for at least 1, 2, 4, 6, 8 or 12 hours. The plasma concentration for Compound (I) may be between 74.08 and 200 ng/mL for at least 1, 2, 4, 6, 8 or 12 hours.

The plasma concentration for Compound (I) may be between 96.00 and 500 ng/mL for at least 1, 2, 4, 6, 8 or 12 hours. The plasma concentration for Compound (I) may be between 96.00 and 400 ng/mL for at least 1, 2, 4, 6, 8 or 12 hours. The plasma concentration for Compound (I) may be between 96.00 and 300 ng/mL for at least 1, 2, 4, 6, 8 or 12 hours. The plasma concentration for Compound (I) may be between 96.00 and 200 ng/mL for at least 1, 2, 4, 6, 8 or 12 hours.

In some embodiments, the plasma concentration for Compound (I) represents an average plasma concentration level for a group of treated subjects and the time period of 1 hour or more begins at any time point following administration. In some embodiments, the plasma concentration for Compound (I) of about 5.04 ng/ml or more for a period of about 1 hour or more represents an average plasma concentration level for a group of treated subjects and the time period of 1 hour or more begins at any time point following administration. In some embodiments, if the average plasma concentration for a group of treated subjects meets a condition, e.g., about 5.04 ng/ml or more for a period of about 1 hour or more, the plasma concentration for the individually treated subject may deviate from the condition. Such deviation is still within the scope of the invention. Accordingly, in some embodiments, the average plasma concentration for a group of treated subjects is about 5.04 ng/ml for a period of about 1 hour or more, wherein the plasma concentration for an individually treated subject is greater than the average plasma concentration for the group of treated subjects. Alternatively, or additionally, in some embodiments, the average plasma concentration for a group of treated subjects is about 5.04 ng/ml for a period of about 1 hour or more, wherein the plasma concentration for an individually treated subject is less than the average plasma concentration for the group of treated subjects.

In some embodiments, the plasma concentration for Compound (I) represents the plasma concentration level for the individually treated subject and the time period of 1 hour or more begins at any time point following administration to that subject. In some embodiments, the plasma concentration for Compound (I) of about 5.04 ng/ml or more for a period of about 1 hour or more represents the plasma concentration level for the individually treated subject and the time period of 1 hour or more begins at any time point following administration to that subject.

The Cmax for administration of Compound (I) may be about 8.30 ng/mL or more. The Cmax for administration of Compound (I) may be at least 8.30, 10.3, 12.2, 15, 20, 25, 30, 35, 40, 45, 50, 60, 70, 80, 90 or 100 ng/mL. The Cmax for administration of Compound (I) may be at least 10.3 ng/mL. The Cmax for administration of Compound (I) may be at least 12.2 ng/mL. The Cmax for administration of Compound (I) may be at least 22.0 ng/mL. The Cmax for administration of Compound (I) may be at least 98.3 ng/mL.

The Cmax for administration of Compound (I) may be about 600 ng/mL or less. The Cmax for administration of Compound (I) may be at most 600, 550, 500, 450, 400, 350, 300, 250, 200, 150 or 100 ng/mL.

The Cmax/Dose for administration of Compound (I) may be about 1.66 ng/mL/mg or more. The Cmax/Dose for administration of Compound (I) may be at least 1.0, 1.1, 1.2, 1.3, 1.4, 1.5, 1.6, 1.66, 1.7, 1.8, 1.9, 2.0, 2.05, 2.1, 2.2, 2.3, 2.4, 2.44, 2.5, 2.6, 2.7, 2.8, 2.9 or 3.0 ng/mL/mg. The Cmax/Dose for administration of Compound (I) may be at least 1.66 ng/mL/mg. The Cmax/Dose for administration of Compound (I) may be at least 2.44 ng/mL/mg. The Cmax/Dose for administration of Compound (I) may be at least 1.96 ng/mL/mg. The Cmax/Dose for administration of Compound (I) may be at least 2.20 ng/mL/mg.

The AUC∞ for administration of Compound (I) may be about 75.6 ng*h/mL or more. The AUC∞ for administration of Compound (I) may be at least 60, 65, 70, 75.6, 75, 80, 85, 89.3, 90, 95, 100, 103, 150, 200, 250, 300, 400, 500, 600, 700, 800, 900 or 1000 ng*h/mL. The AUC∞ for administration of Compound (I) may be at least 89.3 ng*h/mL. The AUC∞ for administration of Compound (I) may be at least 103 ng*h/mL. The AUC∞ for administration of Compound (I) may be at least 201 ng*h/mL. The AUC∞ for administration of Compound (I) may be at least 959 ng*h/mL.

The AUC∞ for administration of Compound (I) may be about 5000 ng*h/mL or less. The AUC∞ for administration of Compound (I) may be at most 5000, 4500, 4000, 3500, 3000, 2500, 2000, 1500, or 1000 ng*h/mL.

The AUC∞/Dose for administration of Compound (I) may be about 15.1 ng*h/mL/mg or more. The AUC∞/Dose for administration of Compound (I) may be at least 10, 11, 12, 13, 14, 15, 15.1, 16, 17, 17.9, 18, 19, 20, 20.6, 21, 22, 23, 24, 25, 26, 27, 28, 29 or 30 ng*h/mL/mg. The AUC∞/Dose for administration of Compound (I) may be at least 17.9 ng*h/mL/mg. The AUC∞/Dose for administration of Compound (I) may be at least 20.6 ng*h/mL/mg. The AUC∞/Dose for administration of Compound (I) may be at least 17.95 ng*h/mL/mg. The AUC∞/Dose for administration of Compound (I) may be at least 21.45 ng*h/mL/mg.

In some embodiments, the plasma concentration for Compound (I) is also about a half of Cmax for administration of Compound (I) or less at about 1 hour prior to sleep time. In some embodiments, the plasma concentration for Compound (I) is further about a quarter of Cmax for administration of Compound (I) or less at about 1 hour prior to sleep time. In some embodiments, the plasma concentration for Compound (I) is also further about a half of 5.04 ng/mL or less at about 1 hour prior to sleep time. In some embodiments, the plasma concentration for Compound (I) is also further about a quarter of 5.04 ng/mL or less at about 1 hour prior to sleep time.

Compound (I) may be administered at least 1, 2, 3, 4, 5, 6, 7, 8, 9, 10 or more times per day. In some embodiments, Compound (I) is administered at least once per day. In some embodiments, the administration of Compound (I) is a multiple daily administration. In some embodiments, Compound (I) is administered at least twice per day.

Compound (I) may be administered at least 1, 2, 3, 4, 5, 6, 7, 8, 9, 10, 11, 12, 13, or 14 or more times per week. In some embodiments, Compound (I) is administered at once per week. In some embodiments, Compound (I) is administered at least twice per week. In some embodiments, Compound (I) is administered at least 3 times per week. In some embodiments, Compound (I) is administered at least 4 times per week.

Compound (I) may be administered at least 1, 2, 3, 4, 5, 6, 7, 8, 9, 10, 11, 12, 13, 14, 15, 16, 17, 18, 19, or 20 or more times per month. Compound (I) may be administered at least 4 times per month.

The methods disclosed herein may further comprise administering one or more additional therapies. The kits and compositions disclosed herein may further comprise one or more additional therapies. The one or more additional therapies may be selected from stimulant, antidepressant, central nervous system depressant, histamine 3 (H3) receptor antagonist, and any other concomitant drugs described herein. In some embodiments, the stimulant is modafinil. In some embodiments, the antidepressant is clomipramine. In some embodiments, the central nervous system depressant is sodium oxybate. In some embodiments, the H3 receptor antagonist is pitolisant.

Examples of the concomitant drug include, but are not limited to, the following. A therapeutic drug for narcolepsy (e.g., methylphenidate, amphetamine, pemoline, phenelzine, protriptyline, sodium oxybate, modafinil, caffeine, pitolisant, solriamfetol), antiobesity drug (amphetamine, benzfetamine, bromocriptine, bupropion, diethylpropion, exenatide, fenfluramine, liothyronine, liraglutide, mazindol, methamphetamine, octreotide, octreotide, orlistat, phendimetrazine, phendimetrazine, phenmetrazine, phentermine, Qnexa (registered trade mark), phenylpropanolamine, pramlintide, propylhexedrine, recombinant leptin, sibutramine, topiramate, zimelidine, zonisamide, Lorcaserin, metformin), acetylcholine esterase inhibitor (e.g., donepezil, rivastigmine, galanthamine, zanapezil, idebenone, tacrine), antidementia agent (e.g., memantine), inhibitor of β-amyloid protein production, secretion, accumulation, aggregation and/or deposition, β-secretase inhibitor (e.g., 6-(4-biphenylyl) methoxy-2-[2-(N,N-dimethylamino)ethyl]tetralin, 6-(4-biphenylyl)methoxy-2-(N,N-dimethyl amino)methyltetralin, 6-(4-biphenylyl)methoxy-2-(N,N-dipropylamino)methyltetralin, 2-(N,N-dimethylamino)methyl-6-(4′-methoxybiphenyl-4-yl)methoxytetralin, 6-(4-biphenylyl) methoxy-2-[2-(N,N-diethylamino)ethyl]tetralin, 2-[2-(N,N-dimethylamino)ethyl]-6-(4′-methylbiphenyl-4-yl)methoxytetralin, 2-[2-(N,N-dimethylamino)ethyl]-6-(4′-methoxy biphenyl-4-yl)methoxytetralin, 6-(2′,4′-dimethoxybiphenyl-4-yl)methoxy-2-[2-(N,N-dimethylamino)ethyl]tetralin, 6-[4-(1,3-benzodioxol-5-yl)phenyl]methoxy-2-[2-(N,N-dimethylamino)ethyl]tetralin, 6-(3′,4′-dimethoxybiphenyl-4-yl)methoxy-2-[2-(N,N-dimethylamino)ethyl]tetralin, an optically active form thereof, a salt thereof and a hydrate thereof, OM99-2 (WO01/00663)), γ-secretase inhibitor, β-amyloid protein aggregation inhibitor (e.g., PTI-00703, ALZHEMED (NC-531), PPI-368 (National Publication of International Patent Application No. 11-514333), PPI-558 (National Publication of International Patent Application No. 2001-500852), SKF-74652 (Biochem. J. (1999), 340(1), 283-289)), β-amyloid vaccine, β-amyloid-degrading enzyme and the like, brain function enhancer (e.g., aniracetam, nicergoline), therapeutic drug for Parkinson's disease [(e.g., dopamine receptor agonist (e.g., L-DOPA, bromocriptine, pergolide, talipexole, pramipexole, cabergoline, amantadine), monoamine oxidase enzyme (MAO) inhibitor (e.g., deprenyl, selegiline, remacemide, riluzole), anticholinergic agent (e.g., trihexyphenidyl, biperiden), COMT inhibitor (e.g., entacapone)], therapeutic drug for amyotrophic lateral sclerosis (e.g., riluzole etc., neurotrophic factor), therapeutic drug for abnormal behavior accompanying progress of dementia, wandering and the like (e.g., sedative, anti-anxiety drug), apoptosis inhibitor (e.g., CPI-1189, IDN-6556, CEP-1347), neuronal differentiation/regenerate promoter (e.g., leteprinim, xaliproden; SR-57746-A), SB-216763, Y-128, VX-853, prosaptide, 5,6-dimethoxy-2-[2,2,4,6,7-pentamethyl-3-(4-methylphenyl)-2,3-dihydro-1-benzofuran-5-yl]isoindoline, 5,6-dimethoxy-2-[3-(4-isopropylphenyl)-2,2,4,6,7-pentamethyl-2,3-dihydro-1-benzofuran-5-yl]isoindoline, 6-[3-(4-isopropylphenyl)-2,2,4,6,7-pentamethyl-2,3-dihydro-1-benzofuran-5-yl]-6,7-dihydro-5H-[1,3]dioxolo[4,5-f]isoindole and an optically active form, salt or hydrate thereof), non-steroidal antiinflammatory agents (meloxicam, tenoxicam, indomethacin, ibuprofen, celecoxib, rofecoxib, aspirin, indomethacin etc.), steroid drug (dexamethasone, hexestrol, cortisone acetate etc.), disease-modifying anti-rheumatic drug (DMARDs), anti-cytokine drug (e.g., TNF inhibitor, MAP kinase inhibitor), therapeutic agent for incontinence, frequent urination (e.g., flavoxate hydrochloride, oxybutynin hydrochloride, propiverine hydrochloride), phosphodiesterase inhibitor (e.g., sildenafil(citrate)), dopamine agonist (e.g., apomorphine), antiarrhythmic drugs (e.g., mexiletine), sex hormone or a derivative thereof (e.g., progesterone, estradiol, estradiol benzoate), therapeutic agent for osteoporosis (e.g., alfacalcidol, calcitriol, elcatonin, calcitonin salmon, estriol, ipriflavone, pamidronate disodium, alendronate sodium hydrate, incadronate disodium), parathyroid hormone (PTH), calcium receptor antagonists, therapeutic drug for insomnia (e.g., benzodiazepines medicament, non-benzodiazepines medicament, melatonin agonist, orexin receptor antagonists), therapeutic drug for schizophrenia (e.g., typical antipsychotic agents such as haloperidol and the like; atypical antipsychotic agents such as clozapine, olanzapine, risperidone, aripiprazole and the like; medicament acting on metabotropic glutamate receptor or ion channel conjugated-type glutamate receptor; phosphodiesterase inhibitor), benzodiazepines medicament (chlordiazepoxide, diazepam, potassium clorazepate, lorazepam, clonazepam, alprazolam etc.), L-type calcium channel inhibitor (pregabalin etc.), tricyclic or tetracyclic antidepressant (imipramine hydrochloride, amitriptyline hydrochloride, desipramine hydrochloride, clomipramine hydrochloride etc.), selective serotonin reuptake inhibitor (fluvoxamine maleate, fluoxetine hydrochloride, citalopram hydrobromide, sertraline hydrochloride, paroxetine hydrochloride, escitalopram oxalate etc.), serotonin-noradrenaline reuptake inhibitor (venlafaxine hydrochloride, duloxetine hydrochloride, desvenlafaxine hydrochloride etc.), noradrenaline reuptake inhibitor (reboxetine mesylate etc.), mirtazapine, trazodone hydrochloride, nefazodone hydrochloride, bupropion hydrochloride, setiptiline maleate, 5-HT_{1A} agonist, (buspirone hydrochloride, tandospirone citrate, osemozotan hydrocloride etc.), 5-HT_{2A} antagonist, 5-HT_{2A} inverse agonist, 5-HT₃ antagonist (cyamemazine etc.), heart non-selective R inhibitor (propranolol hydrochloride, oxprenolol hydrochloride etc.), histamine H₁ antagonist (hydroxyzine hydrochloride etc.), CRY antagonist, other antianxiety drug (meprobamate etc.), tachykinin antagonist (MK-869, saredutant etc.), medicament that acts on metabotropic glutamate receptor, CCK antagonist, β3 adrenaline antagonist (amibegron hydrochloride etc.), GAT-1 inhibitor (tiagabine hydrochloride etc.), N-type calcium channel inhibitor, carbonic anhydrase II inhibitor, NMDA glycine moiety agonist, NMDA antagonist (memantine etc.), peripheral benzodiazepine receptor agonist, vasopressin antagonist, vasopressin V1b antagonist, vasopressin V1a antagonist, phosphodiesterase inhibitor, opioid antagonist, opioid agonist, uridine, nicotinic acid receptor agonist, thyroid hormone (T3, T4), TSH, TRH, MAO inhibitor (phenelzine sulfate, tranylcypromine sulfate, moclobemide etc.), COMT inhibitor (entacapone etc.), therapeutic drug for bipolar disorder (lithium carbonate, sodium valproate, lamotrigine, riluzole, felbamate etc.), cannabinoid CB1 antagonist (rimonabant etc.), FAAH inhibitor, sodium channel inhibitor, anti-ADHD drug (methylphenidate hydrochloride, methamphetamine hydrochloride etc.), therapeutic drug for alcoholism, therapeutic drug for autism, therapeutic drug for chronic fatigue syndrome, therapeutic drug for spasm, therapeutic drug for fibromyalgia syndrome, therapeutic drug for headache, therapeutic drug for quitting smoking, therapeutic drug for myasthenia gravis, therapeutic drug for cerebral infarction, therapeutic drug for mania, therapeutic drug for hypersomnia, therapeutic drug for pain, therapeutic drug for dysthymia, therapeutic drug for autonomic ataxia, therapeutic drug for male and female sexual dysfunction, therapeutic drug for migraine, therapeutic drug for pathological gambler, therapeutic drug for restless legs syndrome, therapeutic drug for substance addiction, therapeutic drug for alcohol-related syndrome, therapeutic drug for irritable bowel syndrome, therapeutic drug for ALS (riluzole etc., neurotrophic factor etc.), therapeutic drug for lipid abnormality such as cholesterol-lowering drug (statin series (pravastatin sodium, atorvastatin, simvastatin, rosuvastatin etc.), fibrate (clofibrate etc.), squalene synthetase inhibitor), therapeutic drug for abnormal behavior or suppressant of dromomania due to dementia (sedatives, antianxiety drug etc.), anti-obesity drug, therapeutic drug for diabetes, therapeutic agent for diabetic complications, therapeutic drug for hypertension, therapeutic drug for hypotension, diuretic, chemotherapeutic agent, immunotherapeutic agent, antithrombotic agent, anti-cancer agent and the like.

Two or more kinds of the above-mentioned concomitant drug may be used in a mixture at an appropriate ratio.

Compound (I) can also be used in combination with biologics (e.g., antibody drug, nucleic acid or nucleic acid derivative, aptamer drug, vaccine preparation), or can be combined with a gene therapy method and the like and applied as a combination therapy, or can also be used in combination with a treatment in psychiatric field without using drugs.

Examples of the treatment method in the psychiatric field without using drug include modified electroconvulsive therapy, deep brain stimulation therapy, repetitive transcranial magnetic stimulation therapy, psychotherapy including cognitive behavioral therapy and the like.

Further disclosed herein are pharmaceutical compositions comprising Compound (I). In some embodiments, the pharmaceutical composition comprises (a) methyl 3-((methylsulfonyl)amino)-2-(((4-phenylcyclohexyl)oxy)methyl)piperidine-1-carboxylate (Compound (I)), or a salt thereof; and (b) a pharmaceutically acceptable carrier therefor, which provides a plasma concentration for Compound (I) of about 5.04 ng/mL or more for about 1 hour or more.

In some embodiments, the pharmaceutical composition provides a Cmax for Compound (I) of about 8.30 ng/mL or more. The pharmaceutical composition may provide a Cmax for Compound (I) of at least 8.30, 10.3, 12.2, 15, 20, 25, 30, 35, 40, 45, 50, 60, 70, 80, 90 or 100 ng/mL. The pharmaceutical composition may provide a Cmax for Compound (I) of at least 10.3 ng/mL. The pharmaceutical composition may provide a Cmax for Compound (I) of at least 12.2 ng/mL. The pharmaceutical composition may provide a Cmax for Compound (I) of at least 22.0 ng/mL. The pharmaceutical composition may provide a Cmax for Compound (I) of at least 98.3 ng/mL.

In some embodiments, the pharmaceutical composition provides a Cmax/Dose for Compound (I) of about 1.66 ng/mL/mg or more. The pharmaceutical composition may provide a Cmax/Dose for Compound (I) of at least 1.0, 1.1, 1.2, 1.3, 1.4, 1.5, 1.6, 1.66, 1.7, 1.8, 1.9, 2.0, 2.05, 2.1, 2.2, 2.3, 2.4, 2.44, 2.5, 2.6, 2.7, 2.8, 2.9 or 3.0 ng/mL/mg. The pharmaceutical composition may provide a Cmax/Dose for Compound (I) of at least 1.66 ng/mL/mg. The pharmaceutical composition may provide a Cmax/Dose for Compound (I) of at least 2.44 ng/mL/mg. The pharmaceutical composition may provide a Cmax/Dose for Compound (I) of at least 1.96 ng/mL/mg. The pharmaceutical composition may provide a Cmax/Dose for Compound (I) of at least 2.20 ng/mL/mg.

In some embodiments, the pharmaceutical composition provides an AUC∞ for Compound (I) of about 75.6 ng*h/mL or more. The pharmaceutical composition may provide an AUC∞ for Compound (I) of at least 60, 65, 70, 75.6, 75, 80, 85, 89.3, 90, 95, 100, 103, 150, 200, 250, 300, 400, 500, 600, 700, 800, 900 or 1000 ng*h/mL. The pharmaceutical composition may provide an AUC∞ for Compound (I) of at least 89.3 ng*h/mL. The pharmaceutical composition may provide an AUC∞ for Compound (I) of at least 103 ng*h/mL. The pharmaceutical composition may provide an AUC∞ for Compound (I) of at least 201 ng*h/mL. The pharmaceutical composition may provide an AUC∞ for Compound (I) of at least 959 ng*h/mL.

In some embodiments, the pharmaceutical composition provides an AUC∞/Dose for Compound (I) of about 15.1 ng*h/mL/mg or more. The pharmaceutical composition may provide an AUC∞ for Compound (I) of at least 10, 11, 12, 13, 14, 15, 15.1, 16, 17, 17.9, 18, 19, 20, 20.6, 21, 22, 23, 24, 25, 26, 27, 28, 29 or 30 ng*h/mL/mg. The pharmaceutical composition may provide an AUC∞ for Compound (I) of at least 17.9 ng*h/mL/mg. The pharmaceutical composition may provide an AUC∞ for Compound (I) of at least 20.6 ng*h/mL/mg. The pharmaceutical composition may provide an AUC∞ for Compound (I) of at least 17.95 ng*h/mL/mg. The pharmaceutical composition may provide an AUC∞ for Compound (I) of at least 21.45 ng*h/mL/mg.

The pharmaceutically acceptable carrier may be a cyclodextrin. The cyclodextrin may be betadex sulfobutyl ether sodium.

In some embodiments, various organic or inorganic carrier substances conventionally used as preparation materials are used as a pharmaceutically acceptable carrier. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Examples of the dosage form of the aforementioned pharmaceutical composition include tablet (including sugar-coated tablet, film-coated tablet, orally disintegrating tablet), capsule (including soft capsule, microcapsule), granule, powder, troche, syrup, emulsion, suspension, films (e.g., orally disintegrable films), injection (e.g., subcutaneous injection, intravenous injection, intramuscular injection, intraperitoneal injection, drip infusion), external preparation (e.g., dermal preparation, ointment), suppository (e.g., rectal suppository, vaginal suppository), pellet, nasal preparation, pulmonary preparation (inhalant), eye drop and the like, which can be respectively safely administered orally or non-orally (e.g., topical, rectal, intravenous administration). These preparations may be a release control preparation (e.g., sustained-release microcapsule) such as an immediate-release preparation, a sustained-release preparation and the like.

In some embodiments, the pharmaceutical composition is formulated for oral administration. In some embodiments, the pharmaceutical composition is formulated for non-oral administration. In some embodiments, the pharmaceutical composition is formulated for intravenous administration, subcutaneous administration, transdermal administration, intradermal administration or transmucosal administration. In some embodiments, the pharmaceutical composition is formulated for intravenous administration. In some embodiments, the pharmaceutical composition is formulated for subcutaneous administration. In some embodiments, the pharmaceutical composition is formulated for transdermal administration.

In some embodiments, Compound (I) is an optical active compound. In some embodiments, Compound (I) is methyl (2R,3S)-3-((methylsulfonyl)amino)-2-(((cis-4-phenylcyclohexyl)oxy)methyl)piperidine-1-carboxylate (Compound A) in any of the methods, the uses and the pharmaceutical compositions disclosed herein. Compound (I) including its salt and its optical active compound may be produced as disclosed in WO2017/135306. In any of the uses and the pharmaceutical compositions disclosed herein, Compound (I) is used in an effective amount thereof. Examples of the effective amount include between about 3 mg and about 500 mg, between about 5 mg and about 300 mg, and between about 5 mg and about 100 mg. The effective amount is preferably between about 5 mg and about 50 mg.

Further disclosed herein are kits comprising Compound (I). In some embodiments, the kit comprises (a) a container comprising methyl 3-((methylsulfonyl)amino)-2-(((4-phenylcyclohexyl)oxy)methyl)piperidine-1-carboxylate (Compound (I)), or a salt thereof, and (b) instructions for administering Compound (I).

The kits disclosed herein may further comprise an additional container comprising saline.

The container may be a glass vial. Alternatively, the container may be a syringe.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “administration” of an agent to a subject includes any route of introducing or delivering the agent to a subject to perform its intended function. Administration can be carried out by any suitable non-oral route, including, but not limited to, intravenously, intramuscularly, intraperitoneally, subcutaneously, and other suitable routes as described herein. Administration includes self-administration and the administration by another.

As used herein, the term “effective amount” or “therapeutically effective amount” refers to a quantity of Compound (I) sufficient to achieve a desired effect or a desired therapeutic effect. In the context of therapeutic applications, the amount of Compound (I) administered to the subject can depend on the type and severity of the disease (e.g., narcolepsy, narcolepsy type 1) or symptom and on the characteristics of the individual, such as general health, age, sex, body weight and tolerance to drugs. The skilled artisan will be able to determine appropriate dosages depending on these and other factors.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “an OX2R agonist” refers to methyl 3-((methylsulfonyl)amino)-2-(((4-phenylcyclohexyl)oxy)methyl)piperidine-1-carboxylate (“Compound (I)”), or a salt thereof. In some embodiments, Compound (I) is methyl (2R,3S)-3-((methylsulfonyl)amino)-2-(((cis-4-phenylcyclohexyl)oxy)methyl)piperidine-1-carboxylate (Compound A).

### EXAMPLES

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example I: Human Study of Safety/Tolerability, Pharmacokinetics and Pharmacodynamics of a Single Dose of an Orexin Type-2 Receptor (OX2R) Agonist in Healthy Subjects and NT1 Patients

This study investigates (i) the safety and tolerability of Compound A (methyl (2R,3S)-3-((methylsulfonyl)amino)-2-(((cis-4-phenylcyclohexyl)oxy)methyl)piperidine-1-carboxylate) when a single dose given as a 9 hour continuous IV infusion is administered to healthy adults, healthy elderly subjects and patients with narcolepsy type 1 (NT1); (ii) the pharmacokinetics of Compound A when a single dose given as a 9 hour continuous IV infusion is administered to healthy adults, healthy elderly subjects and patients with narcolepsy type 1; and (iii) the pharmacodynamics (PD) effects of Compound A, primarily by evaluation of the sleep latency on MWT when a single dose given as a 9 hour continuous IV infusion is administered to patients with narcolepsy type 1.

Study Overview

An overview of the study is shown in Table 1. The treatment schedule for Parts 1 and 2 are shown in Tables 2 and 3, respectively. Subjects re-visit the hospital on Day 7 and the same schedule (after Day −1 and subsequent days) is repeated for the subsequent Dose Levels after washout period of at least one day.

In Part 2, NT1 patients received a single dose of Compound A or placebo administered as a continuous IV infusion over a 9-hour period during the day. A 40-minute Maintenance of Wakefulness Test (MWT) was conducted 4 times during the continuous IV infusion to assess effects on wakefulness in soporific conditions at 2, 4, 6 and 8 hours from start of infusion. Three cohorts of patients were enrolled (n=14 total).

TABLE 1
Study Treatment Groups

| | | Subjects | | | |
|---|---|---|---|---|---|
| Part | Cohort | Sample Size | Study Design | Dosage | Randomization |
| 1¹⁾ | 1²⁾ | Healthy adults | Double-blind, | Compound A 7 mg (Dose | Each Cohort |
| | | n = 8 | Cross-over | Level 1) or placebo | Compound A: 6 |
| | 2 | | | Compound A 14 mg | subjects, |
| | | | | (Dose Level 2) or placebo | Placebo: 2 |
| | 1 | | | Compound A 28 mg | subjects |
| | | | | (Dose Level 3) or placebo | |
| | 2 | | | Compound A 56 mg | |
| | | | | (Dose Level 4) or placebo | |
| | 1 | | | Compound A 112 mg | |
| | | | | (Dose Level 5) or placebo | |
| | 2 | | | Compound A 134.4 mg | |
| | | | | (Dose Level 6) or placebo | |
| | S1 | Healthy adults; | Double-blind, | Compound A 180 mg or | Compound A: 6 |
| | | n = 8 | parallel group | placebo | subjects, |
| | | | | | Placebo: 2 |
| | | | | | subjects |
| | S2 | Healthy adults; | Double-blind, | Compound A 240 mg or | Compound A: 6 |
| | | n = 8 | parallel group | placebo | subjects, |
| | | | | | Placebo: 2 |
| | | | | | subjects |
| | 3 | Healthy elderly; | Double-blind, | Compound A 112 mg or | Compound A: 6 |
| | | n = 8 | parallel group | placebo | subjects, |
| | | | | | Placebo: 2 |
| | | | | | subjects |
| | 4 | Healthy adults | Unblinded | Compound A 112 mg | Compound A: 4 |
| | | (CSF) n = 4 | | | subjects |
| 2¹⁾ | 5 | NT1 patients; | Double-blind; | Compound A 44.8³⁾ mg or | Each period: |
| | | n = 4 | (sponsor-open) | placebo | Compound A: 2 |
| | | | 2 × 2 Cross-over | | subjects, |
| | 6 | NT1 patients; | Double-blind; | Compound A 11.2 mg or | Placebo: 2 |
| | | n = 4 | (sponsor-open) | placebo | subjects |
| | | | 2 × 2 Cross-over | | |
| | 7 | NT1 patients; | Double-blind; | Compound A 5 mg or | Each period: |
| | | n = 6 | (sponsor-open) | placebo | Compound A: 3 |
| | | | 2 × 2 Cross-over | | subjects, |
| | | | | | Placebo: 3 |
| | | | | | subjects |

¹⁾In Part 1, Dose Levels 1, 3 and 5 were tested in the same set of subjects, and similarly, Dose Levels 2, 4 and 6 were evaluated in the different set of the same subjects. In S1 and S2 cohorts, different subjects were used in each cohort. In Part 2, different subjects were used in each Cohort.
²⁾In Cohort 1 Dose Level 1, 2 sentinel subjects were enrolled at first and one subject each was assigned randomly to either Compound A or placebo group to evaluate the safety and tolerability of Compound A. Once the safety and tolerability of these two subjects was evaluated, additional 6 subjects were enrolled and 5 subjects and 1 subject of the 6 subjects were assigned randomly to the Compound A and placebo groups, respectively, to evaluate the safety and tolerability of Compound A.
³⁾In Part 2, the dose level used in Cohort 5 was equal to one-third of the maximum dose of Compound A of which the safety and tolerability had been already confirmed in Part 1 when Cohort 5 was started.

TABLE 2
Part 1: Healthy volunteers study schedule

| | | Treatment Period | | Follow-up | |
|---|---|---|---|---|---|
| | | Study Drug | | Period | |
| | Base- | Administration/ | | Hospital | Washout |
| Screening | line | Sampling | Sampling | Visit | Period* |

| | | | | |
|---|---|---|---|---|
| Day −28 | Day −1 | Day 1 | Day 2 | Day 7 |
| to −2 | | | | |

Hospitalization Period

*Based on the PK results, washout period in addition to the period after dosing to follow-up period was not needed because of short half-life of Compound A.

TABLE 3
Part 2: Narcolepsy type 1 subjects study schedule

| | | Crossover period | | | | Follow-up |
|---|---|---|---|---|---|---|
| | Baseline | Study Drug | | Study Drug | | Period |
| | MWT/ | Administration/ | Sampling/ | Administration/ | | Hospital |
| Screening | PSG | Sampling/MWT | PSG | Sampling/MWT | Sampling | Visit |
| Day −42 | Day −1 | Day 1 | Day 2 | Day 3 | Day 4 | Day 7 |
| to −2* | | | | | | |

Hospitalization Period

*For the subjects receiving prohibited concomitant medications, a washout period will be added during the screening period.

Endpoints

Primary Endpoints

Safety and tolerability: adverse events, vital signs, 12-lead ECG and clinical laboratory tests (hematological tests, blood biochemical tests and urinalysis)

Pharmacokinetics: Concentrations of Compound A in plasma, and CSF, and pharmacokinetics parameters.

Secondary Endpoints

The average sleep latency in the Maintenance Wakefulness Test (MWT)

Exploratory Endpoints

Sleep related parameters in the MWT (sleep latency (SL) in each session).

Daytime sleepiness as assessed by the Karolinska Sleepiness Scale (KSS).

Assessment of cataplexy and other sleep manifestations using a daily diary to collect episodes of cataplexy and sleep symptoms associated with NT1.

Selection of Study Population

Inclusion Criteria:
a. Healthy adult participants and Healthy elderly participants:
   i. Participant weighs at least 50 kg (Healthy adult participants)/40 kg (Healthy elderly participants) and has a body mass index (BMI) from 18.5 to 30 kg/m², inclusive at Screening.
b. Narcolepsy patients:
   i. Patient weighs at least 40 kg inclusive at Screening.
   ii. A diagnosis of narcolepsy Type 1, as defined by the International Classification of Sleep Disorders, Third Edition (ICSD-3).
   iii. HLA narcolepsy test positivity.
   iv. At Day −1, Epworth sleepiness scale (ESS) score ≥10.
   v. Blood pressure <140 systolic and <90 diastolic. The patient may have a history of hypertension and be on antihypertensive medication treatment as long as the BP meets these criteria.

Exclusion Criteria:
a. All Participants:
   i. Participant has a history of drug abuse (defined as any illicit drug use) or a history of alcohol abuse within 2 years prior to the Screening visit.
   ii. Past or current epilepsy, seizure, tremor or the disorders of related symptoms.
   iii. Has a lifetime history of major psychiatric disorder, such as major depressive disorder, bipolar disorder, or schizophrenia.
b. HV (only Cohort 4):
   i. Participant has had CSF collection performed within 14 days prior to Check-in (Day −1).
c. Narcolepsy patients
   i. Medical disorder associated with excessive sleepiness other than narcolepsy (including sleep apnea syndrome).
   ii. Excessive caffeine (>400 mg/day) use one week prior to study.

Subject Characteristics

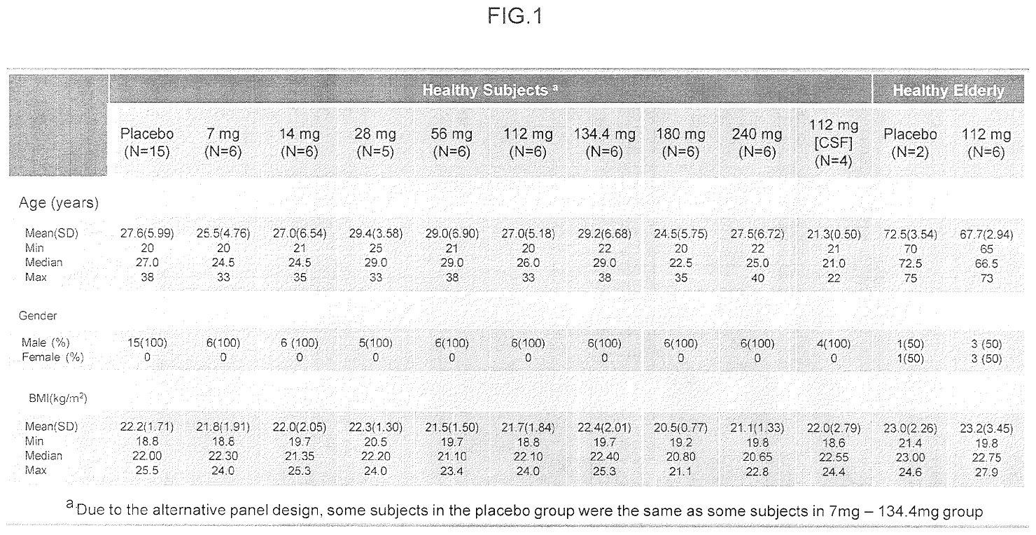

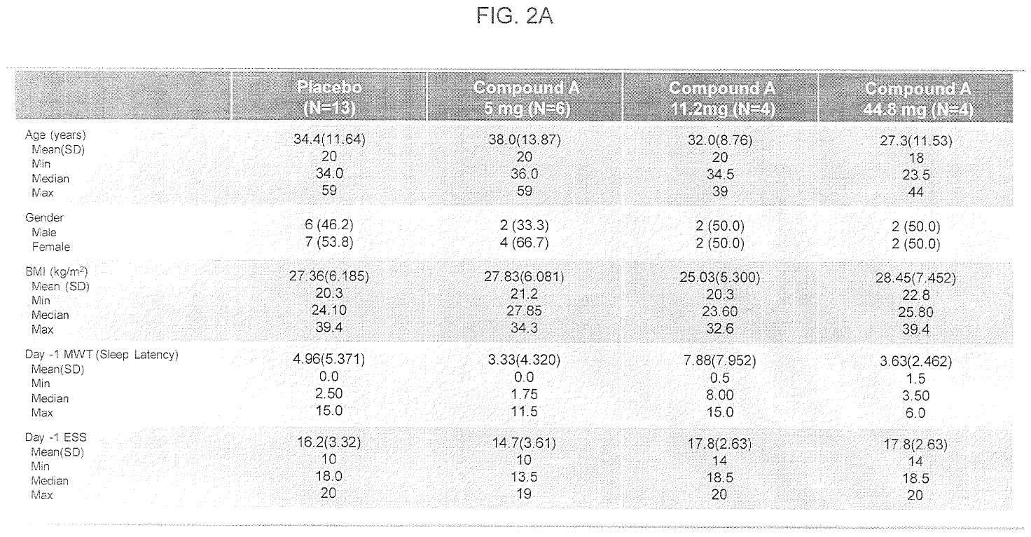

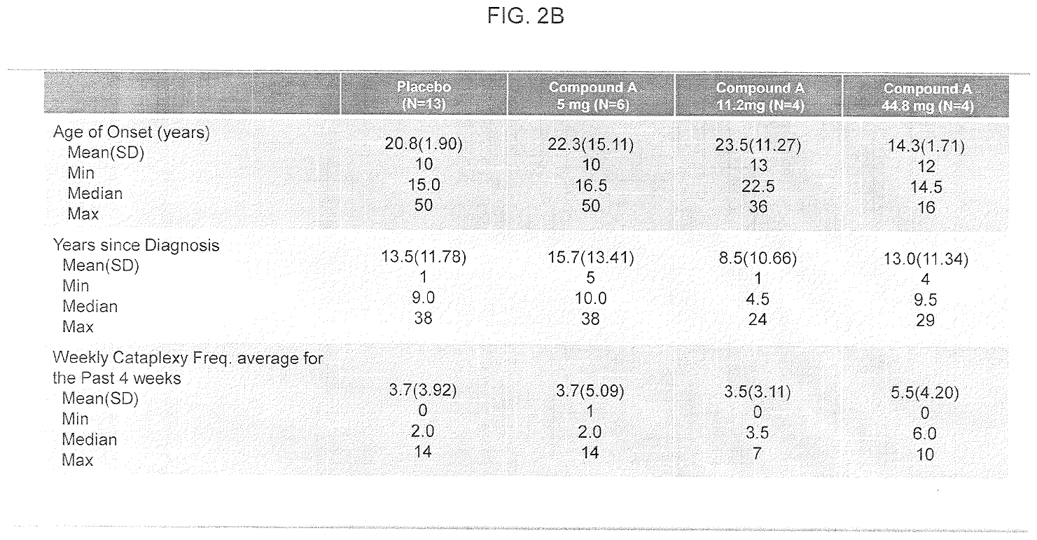

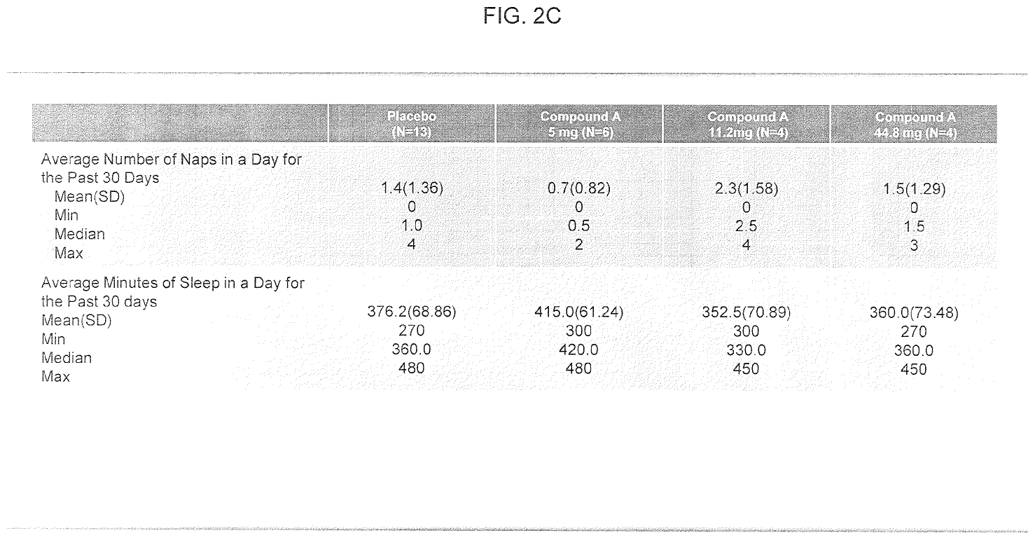

The demographics and baseline characteristics for healthy subjects are shown in FIG. 1 and for subjects with narcolepsy type 1 are shown in FIG. 2A-C. For FIG. 2A-C, due to the crossover design, some subjects with narcolepsy type 1 in the placebo group were the same as some subjects with narcolepsy type 1 in 5 mg-44.8 mg groups. In addition, one subject with narcolepsy type 1 in the 44.8 mg group did not receive a placebo.

Table 4 shows the subject demographics for each cohort.

TABLE 4
Subject Demographics by Cohort

| | Cohort 1, 2 | Cohort S1, S2 | Cohort 3 | Cohort 4 |
|---|---|---|---|---|
| | (N = 16) | (N = 16) | (N = 8) | (N = 4) |
| Age (years) | | | | |
| Mean | 27.8 | 25.7 | 68.9 | 21.3 |
| SD | 5.80 | 5.69 | 3.60 | 0.50 |
| Gender | | | | |
| Male | 16 (100.0) | 16 (100.0) | 4 (50.0) | 4 (100.0) |
| Female | 0 | 0 | 4 (50.0) | 0 |
| BMI (kg/m²) | | | | |
| Mean | 21.94 | 21.24 | 23.16 | 22.03 |
| SD | 1.675 | 1.512 | 3.041 | 2.791 |

### Example I-1: Human Study of Safety/Tolerability of an Orexin 2 Receptor (OX2R) Agonist in Healthy Subjects and NT1 Patients

Investigation of Safety of Compound A

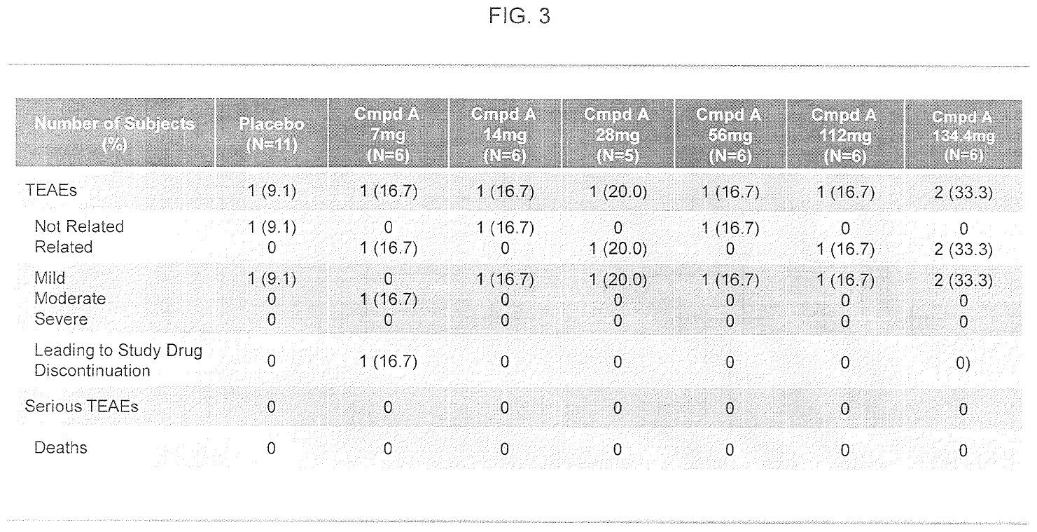

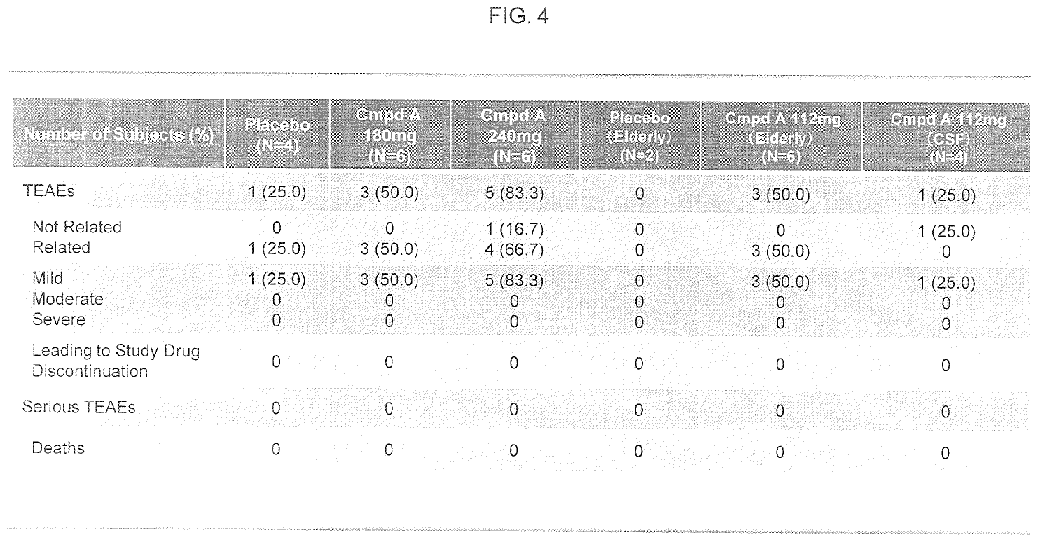

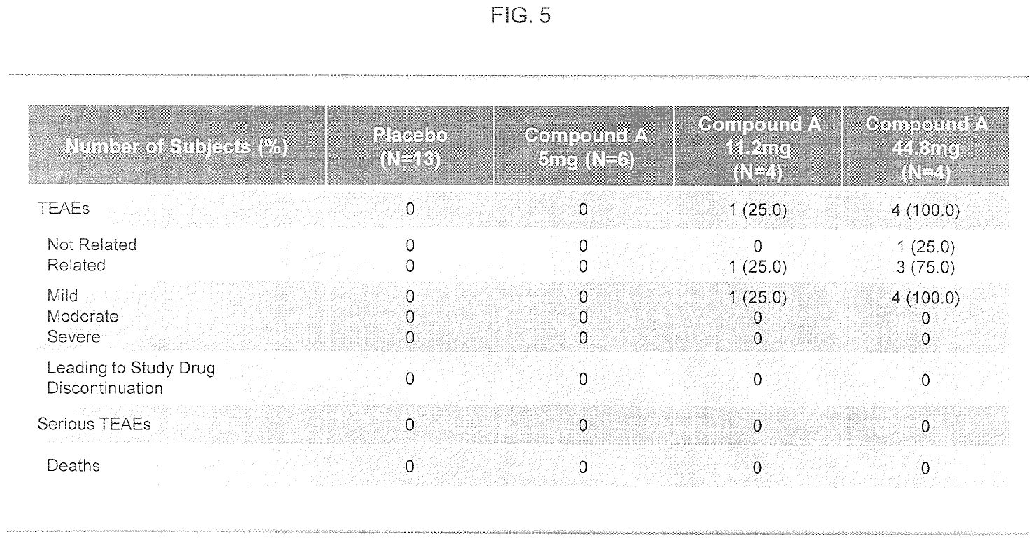

To investigate the safety of Compound A, the treatment-emergent adverse events (TEAEs) for healthy subjects and subjects with narcolepsy type 1 (NT1 patients) treated with Compound A were evaluated. A summary of the TEAEs for healthy subjects from cohorts 1 and 2 is shown in FIG. 3, for healthy subjects from cohorts S1, S2, 3, and 4 is shown in FIG. 4, and for NT1 patients is shown in FIG. 5.

Safety Conclusions

Incidence of AEs in Compound A groups was generally higher than that in the placebo group, but all of the AEs were mild (except 1 moderate influenza) and no serious AEs were observed.

Commonly observed AEs in Compound A groups were BP increased and heart rate increased which are consistent with the mechanism of action. These changes are felt to be consistent with target effects. Incidences for these AEs increased from 134.4 mg in a dose dependent manner and were most clearly observed in the 240 mg group.

At the 112 mg dose, the incidence of BP increase in healthy elderly subjects (aged between 65 and 80 years at the time of signing the consent form) was higher than in healthy adults.

Blood pressure increase, heart rate increase, central nervous system (CNS) related symptoms (euphoric mood, hypnagogic hallucination and logorrhea) and salivary hypersecretion were observed in NT1 at the highest dose (44.8 mg) administered in this population, suggesting a more pronounced effect of Compound A in narcolepsy type 1 patients compared with healthy population at similar doses. A dose dependent effect on BP and heart rate increase was not notable in NT1 subjects at doses up to 44.8 mg, the highest dose tested in this population.

Overall single dose administration of Compound A in a slow infusion over 9 hours up to 240 mg in healthy volunteers, of 112 mg in elderly and up to 44.8 mg in narcolepsy type 1 patients was well-tolerated with no major safety concerns.

### Example I-2A: Compound a Single Dose PK in Healthy Subjects (Cohorts 1, 2, S1 and S2)

Following single IV infusion, mean plasma systemic exposure of Compound A increased approximately dose proportionally over the dose range studied in healthy adult subjects, as evidenced by the lack of apparent difference in AUC∞/Dose and Cmax/Dose of Compound A across doses of 7 to 240 mg. On average, the time to reach the plateau appeared to be approximately 3 to 4 hours after the start of infusion. It was observed that Compound A concentrations at the plateau noticeably decreased around 4 hours post-dose, thus resulting in lower concentrations by the end of infusion. After switching off the IV infusion, Compound A plasma concentrations declined rapidly and in a biphasic manner with a mean terminal disposition phase t_{1/2z} ranging approximately from 3.4 to 5.1 hours across all doses. Mean estimates of plasma clearance of Compound A ranged from 43 to 60 L/h after IV administration. Table 5 provides a summary of plasma concentrations of Compound A after a single IV infusion in healthy subjects. Table 6 provides a summary of pharmacokinetic parameters of Compound A after a single IV infusion in healthy subjects.

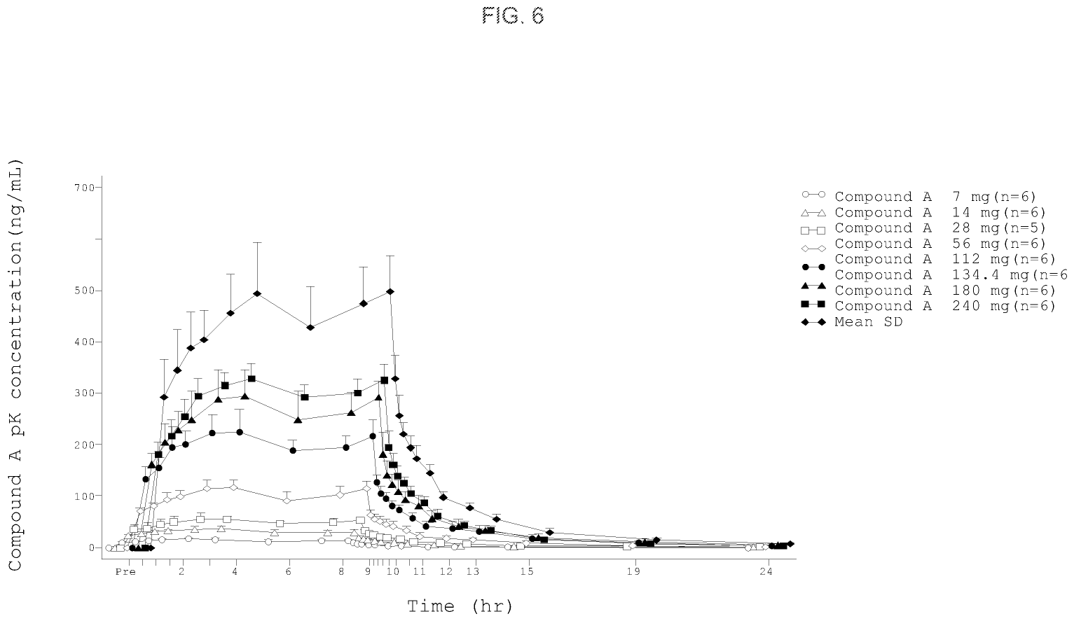

Mean and standard deviation plots of plasma concentrations by Compound A in healthy subjects from cohorts 1, 2, S1 and S2 are presented in FIG. 6.

### Example I-2B: Compound a Single Dose PK in Patients with Narcolepsy (Cohorts 5, 6 and 7)

Summaries of plasma concentrations by visit are presented in Table 7.

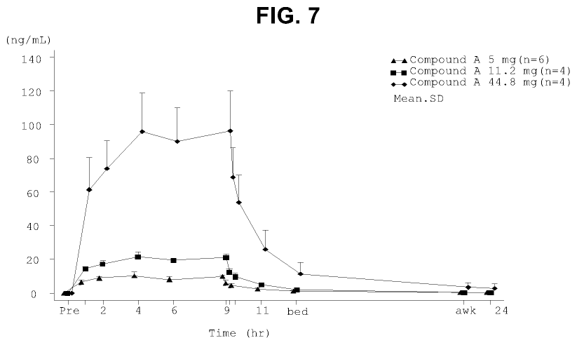

Mean and standard deviation plots of plasma concentrations by Compound A for NT1 are presented in FIG. 7.

Summaries of PK parameters are presented in Table 8.

Following IV administration, mean Compound A concentration-time profiles, systemic exposures and clearance in NT1 patients were consistent with those observed in healthy adults. Mean plasma systemic exposure of Compound A increased approximately dose proportionally over the dose range, as evidenced by the lack of apparent difference in AUC∞/Dose and Cmax/Dose of Compound A across doses of 5 to 44.8 mg. On average, the time to reach the plateau appeared to be approximately 4 hours after the start of infusion. It was observed that Compound A concentrations at the plateau noticeably decreased around 4 hours post-dose, thus resulting in lower concentrations by the end of infusion. After switching off the IV infusion, Compound A plasma concentrations declined rapidly and in a biphasic manner with a mean terminal disposition phase t_{1/2z} ranging approximately from 3.5 to 4.1 hours across the doses. Mean estimates of plasma clearance of Compound A ranged from 50 to 57 L/h after IV administration.

### Example I-2C: Compound a CSF PK in Healthy Subjects (Cohort 4)

Summaries of PK parameters are presented in Tables 9 and 10.

Following a single 9-hour IV infusion of 112 mg to healthy adult subjects in Cohort 4, Compound A plasma concentration-time profile was consistent with that observed in Cohort 1. At 6 hours after the start of infusion, mean ratio CSF/plasma concentration of Compound A was estimated to be about 2.8%.

Table 10 shows the summary of CSF Concentrations by Visit (Cohort 4).

### Example I-3: Sleep Latency in MWT

Investigation of Pharmacodynamics of Compound A

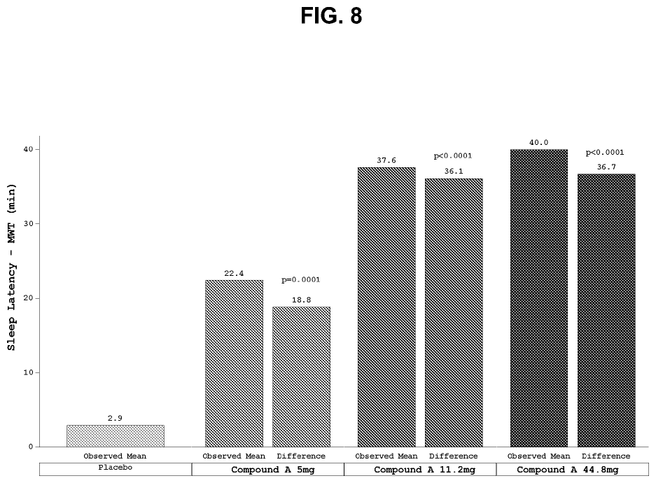

The pharmacodynamics (PD) for sleepiness is shown in FIG. 8, which depicts the average post-dose MWT sleep latency for NT1 patients overall all dose time points. Due to the crossover design, some subjects in the placebo group were the same as some subjects in 5 mg-44.8 mg group.

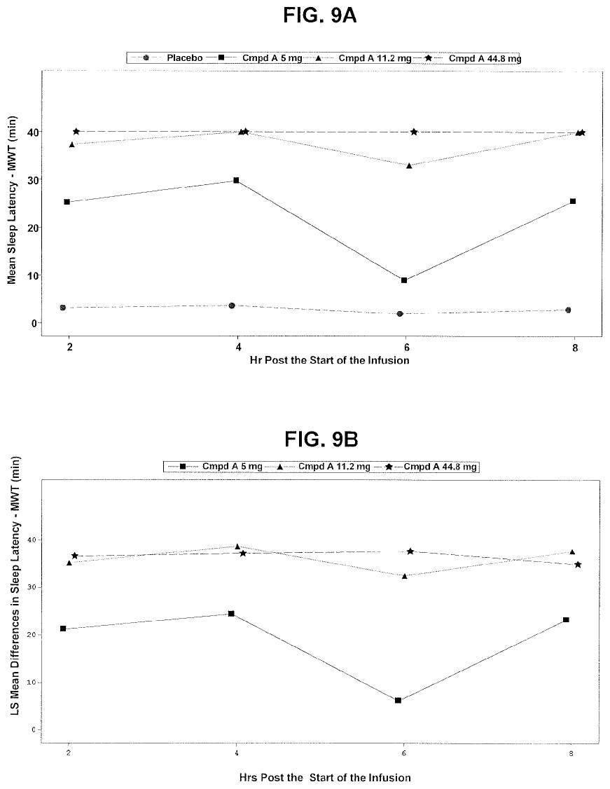

FIG. 9A shows the PD for sleepiness, which is represented as a graph of average post-dose MWT sleep latency versus time points in NT1 patients.

FIG. 9B shows the PD for sleepiness, which is represented as a graph of difference (95% CI) from placebo in post-dose MWT sleep latency versus time points in NT1 patients.

The means (standard deviations [SDs]) of average sleep latency in MWT (min) in the placebo, Compound A 5 mg, Compound A 11.2 mg and Compound A 44.8 mg groups were 2.88 (1.646), 22.38 (10.125), 37.59 (4.813) and 40.00 (0.000), respectively (FIG. 8 and Table 11). The Least Square (LS) mean difference (95% confidence interval [CI]) between Compound A 5 mg minus placebo, Compound A 11.2 mg minus placebo and Compound A 44.8 mg minus placebo were 18.792 (11.987, 25.596), 36.063 (27.729, 44.396) and 36.662 (27.344, 45.979), respectively.

Table 11 shows the summary of Average Sleep Latency in MWT (min, PD Analysis Set).

During 9 hour infusion of Compound A, a significant increase in sleep latency was observed in all active groups compared to placebo, and the maximum sleep latency in a 40 minute-trial MWT, which is 40 min, was achieved in Compound A 11.2 mg (3 out of 4 subjects) and 44.8 mg (4 out of 4 subjects in all four 40-min trials) dosing groups. For session based sleep latency assessment, the lunch session (14:00) showed relatively shorter sleep latency both in placebo and 5 mg active group of Compound A relative to other 3 sessions, while the mean sleep latency in that session was still over 30 min in higher dose groups of Compound A.

Overall, the result supported a clear dose dependent effect of Compound A on wakefulness as measured by the time to sleep onset on the MWT in human narcolepsy type 1 patients.

The summary of sleep related parameters in MWT by visit is presented in Table 12, the average post-dose MWT sleep latency by time points is shown in FIG. 9A and the difference (95% CI) from placebo in post-dose MWT sleep latency by time points is shown in FIG. 9B.

TABLE 12
Summary of Sleep Latency in MWT
by Visit (min, PD Analysis Set)

| | | Compound A | Compound A | Compound A |
|---|---|---|---|---|
| Visit/ | Placebo | 5 mg | 11.2 mg | 44.8 mg |
| Statistics | (N = 13) | (N = 6) | (N = 4) | (N = 4) |
| 10:00 | | | | |
| Mean | 3.15 | 25.25 | 37.38 | 40.00 |
| SD | 2.258 | 16.443 | 5.250 | 0.000 |
| 95% CI | NA | 10.182, 32.318 | 21.695, 48.805 | 21.609, 51.752 |
| 12:00 | | | | |
| Mean | 3.58 | 29.75 | 40:00 | 40.00 |
| SD | 3.252 | 15.145 | 0.000 | 0.000 |
| 95% CI | NA | 15.152, 33.681 | 27.403, 50.097 | 24.508, 49.967 |
| 14:00 | | | | |
| Mean | 1.92 | 8.92 | 33.00 | 40.00 |
| SD | 1.913 | 4.944 | 14.000 | 0.000 |
| 95% CI | NA | −1.403, 13.736 | 23.229, 41.771 | 27.441, 47.963 |
| 16:00 | | | | |
| Mean | 2.88 | 25.58 | 40.00 | 40.00 |
| SD | 2.518 | 16.402 | 0.000 | 0.000 |
| 95% CI | NA | 12.895, 33.772 | 24.966, 50.534 | 20.872, 49.243 |

CI = confidence interval,
NA = not applicable,
SD = standard deviation
The start time of infusion was 7:45.
Note:
95% CI is the 2-sided 95% CI for the Least Square mean difference between the Compound A and placebo from ANOVA models.

This study demonstrated that an OX2R agonist, such as Compound A, had significant wake-promoting effects in NT1 patients. Doses of 5 mg (n=6), 11.2 mg (n=4), and 44.8 mg (n=4) resulted in a dose-dependent effect with a mean sleep latency of 22.4, 37.6, and 40 min, respectively, compared to a combined mean of 2.88 min in the placebo crossover period. This is significantly greater (p<0.001) than the placebo. Also, the lower bounds of the 95% Confidence Interval (CI) for the difference between Compound A and placebo are all above 3 min. The posterior probability of a greater than 3 min placebo-adjusted drug effect is above 95%.

### Example I-4: Subjective Daytime Sleepiness as Assessed by KSS

Daytime sleepiness as assessed by KSS at predose and hourly until 11 hours after the start of infusion is presented in Table 13 as summary, in Table 14 as ANOVA and in Table 15 as ANOVA of change from baseline.

KSS ratings were lower (greater alertness) in the Compound A group versus pooled placebo group at all time points taken over 9 hour study drug infusion period. The LS mean difference (95% CI) of KSS during the 9 hour of infusion between Compound A 5 mg and placebo, Compound A 11.2 mg and placebo and Compound A 44.8 mg and placebo ranged from −3.500 ([−6.003, −0.997], [−6.280, −0.720] and [−6.511, −0.489] respectively at 2 hour, 6 hour and 7 hour after the start of infusion) to −0.667 (−2.641, 1.307), from −4.000 (−7.331, −0.669) to −1.500 (−3.439, 0.439) and from −6.947 (−9.139, −4.756) to −2.868 (−6.851, 1.114), respectively, though the difference became less clear one hour after the end of infusion in between and active drug groups in this analysis. KSS results were consistent with the results on the MWT for time to sleep onset.

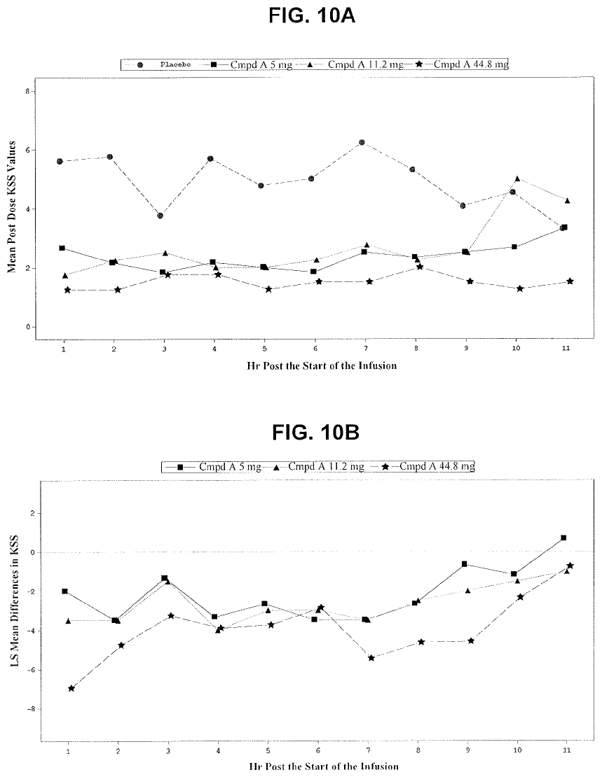

The post-dose KSS observed values by time points are shown in FIG. 10A and the difference from placebo in post-dose KSS by time points is shown in FIG. 10B.

### Example I-5: Additional Exploratory PD Endpoints

“Number of Times of a Cataplexy Episode” is presented in Tables 16 and 17.

One patient included in cohort 7 had a high frequency of cataplexy that may have been due to rebound. This data skewed the overall data on the number of cataplexy episodes in this small sample size study. It should be noted that the frequency of cataplexy compared to baseline frequency of this same subject was remarkably improved during the infusion of Compound A but was reported as almost the same as baseline during placebo infusion (Table 16), which may suggest that the single dose of Compound A was effective in suppressing cataplexy during the infusion, but cataplexy returned once the drug concentration was below a certain level. Number of Times of a Cataplexy Episode in NT1 Patients (Cohorts 5, 6, 7) is shown in Table 17.

TABLE 16
Number of Events of Cataplexy per Week in one particular patient

| | | During | Post | During | Post |
|---|---|---|---|---|---|
| | | Placebo | Placebo | Cmpd A | Cmpd A |
| Screening | Baseline | infusion | infusion | infusion | infusion |
| 2 | 808 | 83 | 65 | 0 | 58 |

TABLE 17
Number of Times of a Cataplexy Episode in NT1 Patients (Cohorts 5, 6, 7) (PD Analysis Set)
Number of Times of a Cataplexy Episode

| | | | Cmpd A | | Cmpd A | | Cmpd A | |
|---|---|---|---|---|---|---|---|---|
| | Placebo | | 5 mg | | 11.2 mg | | 44.8 mg | |
| | (N = 13) | | (N = 6) | | (N = 4) | | (N = 4) | |
| | During | After End | During | After End | During | After End | During | After End |
| | Infusion | of Infusion | Infusion | of Infusion | Infusion | of Infusion | Infusion | of Infusion |
| Mean (SD) | 6.9 (22.92) | 5.6 (17.88) | 0.0 (0.00) | 10.2 (23.45) | 0.8 (1.50) | 0.0 (0.00) | 0.0 (0.00) | 0.8 (1.50) |
| Median | 0.0 | 0.0 | 0.0 | 0.5 | 0.0 | 0.0 | 0.0 | 0.0 |
| Min, Max | 0, 83 | 0, 65 | 0, 0 | 0, 58 | 0, 3 | 0, 0 | 0, 0 | 0, 3 |

| | | | | |
|---|---|---|---|---|
| Subjects without | 7 (53.8) | 3 (50.0) | 3 (75.0) | 3 (75.0) |
| any Events N (%) | | | | |

Pharmacokinetic/Pharmacodynamic Conclusions

Following a single 9-hour IV infusion, the plateau of Compound A plasma concentration appeared to have been reached within 3 to 4 hours after the start of infusion across all dose levels studied.

After the IV infusion ended, Compound A plasma concentrations declined rapidly and in a biphasic manner with a mean terminal disposition phase t_{1/2z} ranging approximately from 3.4 to 5.1 hours.

Mean estimates of plasma clearance of Compound A ranged from 43 to 60 L/h in healthy subjects.

There was no apparent difference in AUC∞/Dose and Cmax/Dose of Compound A across doses of 7 to 240 mg indicating that Compound A exhibits linear pharmacokinetics.

In healthy elderly subjects, Compound A plasma mean exposures were on average approximately 18%-27% higher than those of the younger healthy adults, which was consistent with the observed 20% reduction in Compound A clearance when compared to younger healthy subjects.

Mean ratio CSF/plasma concentration of Compound A was estimated to be about 2.8%.

Systemic exposures and clearance of Compound A in NT1 patients were comparable with those observed in healthy adults.

Trend for the observed BP and pulse rate increases in healthy subjects and Compound A concentration was observed. Trend was less apparent in patients with narcolepsy as they were treated with the lower dose levels.

Mean MWT sleep latency was 40 minutes and 37.59 minutes in the 44.8 and 11.2 mg dose groups, respectively, vs. 2.88 minutes for the pooled placebo group.

Mean MWT sleep latency was 22.38 minutes in the 5 mg group vs 2.88 minutes for pooled placebo group. A dose response for Compound A was observed for effect on sleep latency on the MWT.

Mean reduction in KSS from baseline was greater for Compound A vs placebo, indicating more alertness with Compound A. KSS results were correlated with plasma concentrations and consistent with MWT assessments. While less clear than the results on the MWT, some dose response was observed.

### Example II: Human Multi Study to Evaluate Safety, Tolerability, Pharmacokinetics and Pharmacodynamics of Multiple Doses of an Orexin Type-2 Receptor (OX2R) Agonist in NT1 Patients

The purpose of the study was to evaluate the safety, tolerability, pharmacokinetics and pharmacodynamics of Compound A after multiple IV administration in patients with NT1. NT1 patients were evaluated in Cohort B1 and B2 (Table 19). Compound A or placebo were administered via IV infusion over 9 hours once daily for 7 days. A total of 13 patients with NT1 were treated with Compound A or placebo (4, 4, and 5 subjects in the placebo, Compound A 11 mg, and Compound A 44 mg groups, respectively). In this part, potential efficacy of Compound A was evaluated with MWT, ESS, KSS, night-time PSG (NPSG), cataplexy frequency assessment, and PGI-C as an exploratory PD assessment.

TABLE 19
Outline of the Study Parts and Dosing Cohorts

| Subject | Cohort/Panel | Daily dose level | Dosing regimen |
|---|---|---|---|
| NT1 patients | B1 | 11 mg | IV infusion over 9 |
| | B2 | 44 mg | hours for 7 days |

Abbreviations: IV, intravenous;
NT1, narcolepsy type 1

Overview of Key Study Procedures

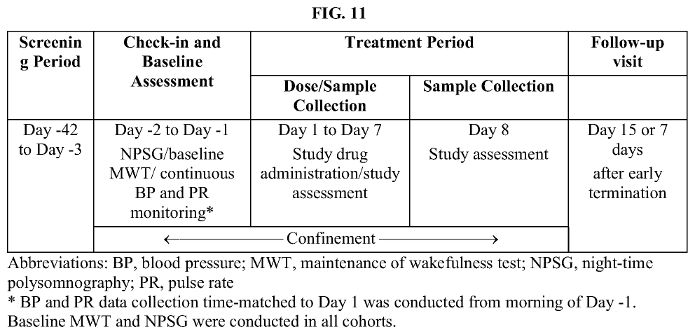

An overview of study schedule and PD testing in NT1 Patients is illustrated in FIG. 11. After screening, patients eligible for these parts had to discontinue any medication for narcolepsy including medications used for EDS or cataplexy. The medications had to be stopped for a minimum of 7 days or at least 5 half-lives of each medication, whichever was longer, before the first day of dosing (Day 1). As for a subject having possibility of getting injured due to severe cataplexy, the subject might have been confined to the site before Day −2 at a discretion of the investigator. In all cohorts of these parts, subjects underwent NPSG on Day −2 and baseline MWT sessions 4 times on Day −1 (at around 10:00, 12:00, 14:00, and 16:00). Study drug dosing via IV infusion was started at around 08:00. MWT was conducted on Day 1 at around 10:00, 12:00, 14:00, and 16:00. Subjects were allowed to take a nap on the days with no MWT assessment. At night of Day 6, the subjects underwent NPSG once again to evaluate the effect of multiple daytime dosing on night-time sleep structure. On Day 7, the subjects underwent MWT at the same hours as on Day −1. Cataplexy was to be evaluated by using the self-recorded sleep diary. The subjects were discharged from the study site on Day 8.

Main Criteria for Selection of Study Population

Subject eligibility was confirmed according to the following criteria

Inclusion Criteria:
The patient must be aged 18 to 80 years, inclusive, at the time of informed consent.
The patient weighs at least 40 kg inclusive at Screening.
The patient must have a diagnosis of NT1, as defined by the International Classification of Sleep Disorders, Third Edition (ICSD-3).
The patient's Epworth sleepiness scale (ESS) is ≥10 at baseline.
HLA narcolepsy test positivity.
Have ≥3 episodes of cataplexy/week reported during the screening period.

Exclusion Criteria:
The patients consume excessive amounts, defined as greater than 6 servings (1 serving is approximately equivalent to 120 mg of caffeine) of coffee, tea, cola, energy drinks, or other caffeinated beverages per day.
The patients have a moderate to severe substance use disorder.
The patients have a risk of suicide according to endorsement of item 4 or 5 with Screening/Baseline visit C-SSRS or has made a suicide attempt in the previous 6 months.
The patients have a lifetime history of major psychiatric disorder, such as bipolar disorder or schizophrenia. Subject who has history of major depressive disorder (MDD) may be included but a subject having active MDD currently or in the past 6 months is excluded.
The patients experienced sleep wake cycle disturbance with external factors such as irregular work hours.

### Example II-1: Multiple-Dose PK of Intravenous Compound a in Patients with NT1

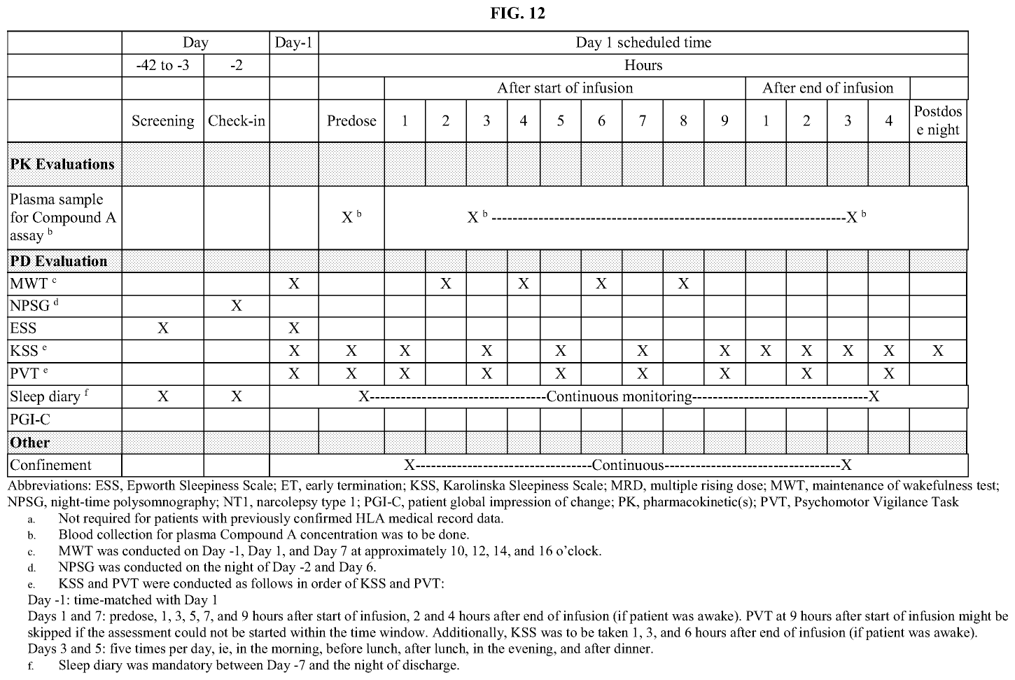

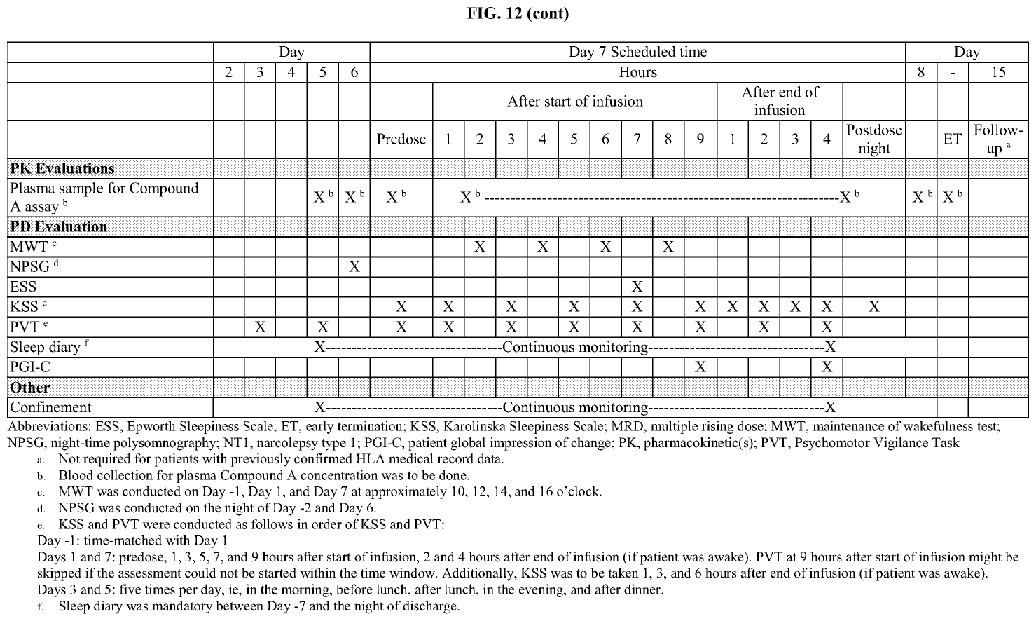

Blood samples used for PK analysis of Compound A was collected according to Table 20. See also FIG. 12 for whole schedules of study procedures.

TABLE 20
Blood Sampling for Pharmacokinetic analysis (NT1 Patients)

| Analyzed | | Study | |
|---|---|---|---|
| substances | Samples | date | Blood sampling time |
| Compound | Plasma | Days 1-8 | B1 and B2 |
| A | | | Days 1 and 7: Predose, 1, 2, 4, 6, |
| | | | and 9 hours after start of infusion, |
| | | | 0.17, 0.5, 2, 6, 10, and 15 hours |
| | | | after end of infusion. |
| | | | B1 and B2 |
| | | | Days 5 and 6: Predose, 9 hours after |
| | | | start of infusion |

Summaries of plasma concentrations of Compound A are presented in Table 21. Mean and standard deviation plots of plasma Compound A concentrations on Day 1 and Day 7 are presented in FIGS. 13A and B, respectively. Summaries of PK parameters of Compound A on Day 1 and Day 7 are presented in Table 22 and Table 23 respectively. Based on R_{ac(AUC)} and R_{ac(Cmax)} values of approximately 1, no drug accumulation with QD dosing after a 9-hour IV infusion daily for 7 days was observed.

TABLE 21
Summary of Plasma Concentration by Visit by Treatment Group (Cohort B1, B2) (PK set)

| | | Visit | | | | | | | | | | |
|---|---|---|---|---|---|---|---|---|---|---|---|---|
| | | Predose | [symbol] | [symbol] | [symbol] | [symbol] | [symbol] | [symbol] | [symbol] | [symbol] | [symbol] | [symbol] |
| Analyte/ | | at Day | at Day | at Day | at Day | at Day | at Day | at Day | at Day | at Day | at Day | at Day |
| Treatment | Statistics | 1 | 1 | [symbol] | [symbol] | [symbol] | [symbol] | [symbol] | [symbol] | [symbol] | [symbol] | [symbol] |
| Cmpd A (ng/mL) | | | | | | | | | | | | |
| Cmpd A 11 mg | N | [symbol] | [symbol] | [symbol] | [symbol] | [symbol] | [symbol] | [symbol] | [symbol] | [symbol] | [symbol] | [symbol] |
| | Mean | [symbol] | [symbol] | [symbol] | [symbol] | [symbol] | [symbol] | [symbol] | [symbol] | [symbol] | [symbol] | [symbol] |
| | SD | [symbol] | [symbol] | [symbol] | [symbol] | [symbol] | [symbol] | [symbol] | [symbol] | [symbol] | [symbol] | [symbol] |
| Cmpd A 44 mg | N | [symbol] | [symbol] | [symbol] | [symbol] | [symbol] | [symbol] | [symbol] | [symbol] | [symbol] | [symbol] | [symbol] |
| | Mean | [symbol] | [symbol] | [symbol] | [symbol] | [symbol] | [symbol] | [symbol] | [symbol] | [symbol] | [symbol] | [symbol] |
| | SD | [symbol] | [symbol] | [symbol] | [symbol] | [symbol] | [symbol] | [symbol] | [symbol] | [symbol] | [symbol] | [symbol] |

[symbol]

| | | Visit | | | | | | | | | | |
|---|---|---|---|---|---|---|---|---|---|---|---|---|
| | | [symbol] | [symbol] | [symbol] | [symbol] | [symbol] | [symbol] | [symbol] | [symbol] | [symbol] | [symbol] | [symbol] |
| Analyte/ | | at Day | at Day | at Day | at Day | at Day | at Day | at Day | at Day | at Day | at Day | at Day |
| Treatment | Statistics | [symbol] | [symbol] | [symbol] | [symbol] | [symbol] | [symbol] | [symbol] | [symbol] | [symbol] | [symbol] | [symbol] |
| Cmpd A (ng/mL) | | | | | | | | | | | | |
| Cmpd A 11 mg | N | [symbol] | [symbol] | [symbol] | [symbol] | [symbol] | [symbol] | [symbol] | [symbol] | [symbol] | [symbol] | [symbol] |
| | Mean | [symbol] | [symbol] | [symbol] | [symbol] | [symbol] | [symbol] | [symbol] | [symbol] | [symbol] | [symbol] | [symbol] |
| | SD | [symbol] | [symbol] | [symbol] | [symbol] | [symbol] | [symbol] | [symbol] | [symbol] | [symbol] | [symbol] | [symbol] |
| Cmpd A 44 mg | N | [symbol] | [symbol] | [symbol] | [symbol] | [symbol] | [symbol] | [symbol] | [symbol] | [symbol] | [symbol] | [symbol] |
| | Mean | [symbol] | [symbol] | [symbol] | [symbol] | [symbol] | [symbol] | [symbol] | [symbol] | [symbol] | [symbol] | [symbol] |
| | SD | [symbol] | [symbol] | [symbol] | [symbol] | [symbol] | [symbol] | [symbol] | [symbol] | [symbol] | [symbol] | [symbol] |

[symbol]

Visit

| | | [symbol] | [symbol] | [symbol] | [symbol] | [symbol] | [symbol] |
|---|---|---|---|---|---|---|---|
| Analyte/ | | at Day | at Day | at Day | at Day | at Day | at Day |
| Treatment | Statistics | [symbol] | [symbol] | [symbol] | [symbol] | [symbol] | [symbol] |
| Cmpd A (ng/mL) | | | | | | | |
| Cmpd A 11 mg | N | [symbol] | [symbol] | [symbol] | [symbol] | [symbol] | [symbol] |
| | Mean | [symbol] | [symbol] | [symbol] | [symbol] | [symbol] | [symbol] |
| | SD | [symbol] | [symbol] | [symbol] | [symbol] | [symbol] | [symbol] |
| Cmpd A 44 mg | N | [symbol] | [symbol] | [symbol] | [symbol] | [symbol] | [symbol] |
| | Mean | [symbol] | [symbol] | [symbol] | [symbol] | [symbol] | [symbol] |
| | SD | [symbol] | [symbol] | [symbol] | [symbol] | [symbol] | [symbol] |

Note:

[symbol]

[symbol]

indicates data missing or illegible when filed

TABLE 22
Summary of Pharmacokinetic Parameters of Compound
A (Cohort B1, B2) on Day 1 (PK set)

| | | Statistics | | | | | |
|---|---|---|---|---|---|---|---|
| | | | | N of Geo | | | |
| Variable | Treatment | N | Mean | Mean | Geo Mean | SD | CV |
| AUClast (h*ng/mL) | Cmpd A 11 mg | 4 | 164.5 | 4 | 163.7 | 18.806 | 11.4 |
| | Cmpd A 44 mg | 5 | 69 [symbol] .0 | 5 | 682.1 | 126.44 | 1 [symbol] .2 |
| AUCinf (h*ng/mL) | Cmpd A 11 mg | 4 | 166.8 | 4 | 165.9 | 19.397 | 11. [symbol] |
| | Cmpd A 44 mg | 5 | 696.8 | 5 | [symbol] 87.9 | 126.8 [symbol] | 18.2 |
| AUCtau (h*ng/mL) | Cmpd A 11 mg | 4 | 165.0 | 4 | 164.2 | 18.257 | 11.1 |
| | Cmpd A 44 mg | 5 | 69 [symbol] .2 | 5 | [symbol] 82.3 | 125.81 | 1 [symbol] .3 |
| Cmax (ng/mL) | Cmpd A 11 mg | 4 | 18.28 | 4 | 18.25 | 1.1026 | 6.0 |
| | Cmpd A 44 mg | 5 | 77. [symbol] 8 | 5 | 7 [symbol] .20 | 15.834 | 20.4 |
| Ceoi (ng/mL) | Cmpd A 11 mg | 4 | 17. [symbol] 3 | 4 | 17.39 | 1. [symbol] 842 | 7.4 |
| | Cmpd A 44 mg | 5 | 72.04 | 5 | 7 [symbol] .20 | 16.79 [symbol] | 26.1 |

| Variable | Treatment | N | Mean | SD |
|---|---|---|---|---|
| tmax (h) | Cmpd A 11 mg | 4 | 5. [symbol] 50 | 2.4557 |
| | Cmpd A 44 mg | 5 | 4.926 | 2.1957 |

| Variable | Treatment | N | Mean | SD | CV |
|---|---|---|---|---|---|
| t½z (h) | Cmpd A 11 mg | 4 | [symbol] .300 | 2.3149 | 39.8 |
| | Cmpd A 44 mg | 5 | 3.2 [symbol] 2 | 0.50 [symbol] 0 | 1 [symbol] .0 |
| Vax (L) | Cmpd A 11 mg | 4 | 129.6 | 2 [symbol] .534 | 22.8 |
| | Cmpd A 44 mg | 5 | 135.0 | 39.487 | 21. [symbol] |
| Vz (L) | Cmpd A 11 mg | 4 | 309.8 | 1 [symbol] 2.92 | 33.2 |
| | Cmpd A 44 mg | 5 | 316.2 | 78.270 | 24.8 |
| CL (L/h) | Cmpd A 11 mg | 4 | 66. [symbol] 3 | 7. [symbol] 390 | 11.7 |
| | Cmpd A 44 mg | 5 | 64.75 | 11.263 | 17.4 |
| Cmax D (ng/mL/mg) | Cmpd A 11 mg | 4 | 1.660 | 0. [symbol] 00 | [symbol] .1 |
| | Cmpd A 44 mg | 5 | 1.7 [symbol] 2 | 0.36129 | 20.5 |
| AUCtau D (h*ng/mL/mg) | Cmpd A 11 mg | 4 | 15.00 | 1.6432 | 11.0 |
| | Cmpd A 44 mg | 5 | 15.70 | 2.8749 | 18.3 |

[symbol]

indicates data missing or illegible when filed

TABLE 23
Summary of Pharmacokinetic Parameters of Compound A (Cohort B1, B2) on Day 7 (PK set)

| Variable | Treatment | N | Mean | N of Geo Mean | Geo Mean | SD | CV |
|---|---|---|---|---|---|---|---|
| AUClast (h*ng/mL) | Cmpd A 11 mg | 4 | [symbol] 83. [symbol] | 4 | 182.4 | 23.000 | [symbol] .5 |
| | Cmpd A 44 mg | 5 | 728. [symbol] | 5 | 7 [symbol] .7 | 102.26 | [symbol] .0 |
| AUCtau (h*ng/mL) | Cmpd A 11 mg | 4 | 183.8 | 4 | 182.7 | 2 [symbol] . [symbol] | 12.3 |
| | Cmpd A 44 mg | 5 | 728.6 | 5 | 722.7 | 102.28 | [symbol] .0 |
| Cmax (ng/mL) | Cmpd A 11 mg | 4 | 20. [symbol] | 4 | 20.49 | 1. [symbol] | 5.1 |
| | Cmpd A 44 mg | 5 | 81. [symbol] | 5 | 81.16 | 11.090 | [symbol] |
| Cmin (ng/mL) | Cmpd A 11 mg | 4 | 0.3258 | 3 | 0.4061 | 0.26019 | 79.9 |
| | Cmpd A 44 mg | 5 | 1.327 | 5 | 1.24 [symbol] | 0.5 [symbol] 62 | 38.4 |
| Ceoi (ng/mL) | Cmpd A 11 mg | 4 | 17.75 | 4 | 17.68 | 1.7176 | 9.7 |
| | Cmpd A 44 mg | 5 | 74.44 | 5 | 73.20 | 14.980 | 20.1 |

| Variable | Treatment | N | Mean | SD | CV |
|---|---|---|---|---|---|
| PTR | Cmpd A 11 mg | 3 | 57.07 | 32.637 | 57.2 |
| | Cmpd A 44 mg | 5 | 70.28 | [symbol] .577 | 44.9 |
| Rac AUC | Cmpd A 11 mg | 4 | 1.118 | 0.14637 | [symbol] .1 |
| | Cmpd A 44 mg | 5 | 1.062 | [symbol] | 6.2 |
| Rac Cmax | Cmpd A 11 mg | 4 | 1.120 | 0.1305 [symbol] | 11.6 |
| | Cmpd A 44 mg | 5 | 1.070 | 0.10144 | 9.5 |

Note:
PTR of one subject d [symbol] d 11 mg was missing because Cmin of the subject was zero.

| Variable | Treatment | N | Mean | SD |
|---|---|---|---|---|
| tmax (h) | Cmpd A 11 mg | 4 | 5.165 | 2.4461 |
| | Cmpd A 44 mg | 5 | 4.930 | 2.1931 |

| Variable | Treatment | N | Mean | SD | CV |
|---|---|---|---|---|---|
| t½z (h) | Cmpd A 11 mg | 4 | 3.595 | [symbol] .3100 | 33.7 |
| | Cmpd A 44 mg | 5 | 4.222 | [symbol] .7716 | 18.3 |
| V [symbol] (L) | Cmpd A 11 mg | 4 | 123.3 | [symbol] .834 | 27.4 |
| | Cmpd A 44 mg | 5 | 134.5 | 26.527 | [symbol] 9.7 |
| Vx (L) | Cmpd A 11 mg | 4 | 313.0 | 110.33 | 35.2 |
| | Cmpd A 44 mg | 5 | 375.2 | 55.980 | 22.9 |
| CL (L/h) | Cmpd A 11 mg | 4 | 60.60 | 7.5242 | 12.4 |
| | Cmpd A 44 mg | 5 | [symbol] 1.40 | 8.0302 | 14.6 |
| Cmax D ( [symbol] g/mL/mg) | Cmpd A 11 mg | 4 | 1.868 | [symbol] 51 | 9.2 |
| | Cmpd A 44 mg | 5 | 1.858 | 0.25103 | 13.6 |
| AUCtau D (h*ng/mL/mg) | Cmpd A 11 mg | 4 | 16.70 | 2.8412 | 12.2 |
| | Cmpd A 44 mg | 5 | 16.56 | 2.32 [symbol] | 14.0 |

[symbol]

indicates data missing or illegible when filed

### Example II-2: MWT

The MWT is a validated objective measure that evaluates a person's ability to remain awake under soporific conditions for a defined period of time. As there is no biological measure of wakefulness, this was measured indirectly by the inability or delayed tendency to fall asleep. This tendency to fall asleep was measured via EEG-derived sleep latency in MWT.

On Day −1, Day 1 and Day 7, 40-minute session (1 session) of MWTs was performed 4 times (approximately at 10:00, 12:00, 14:00, and 16:00) per day. Sleep latency of each session was recorded. Subjects were required to stay awake during an interval between sessions.

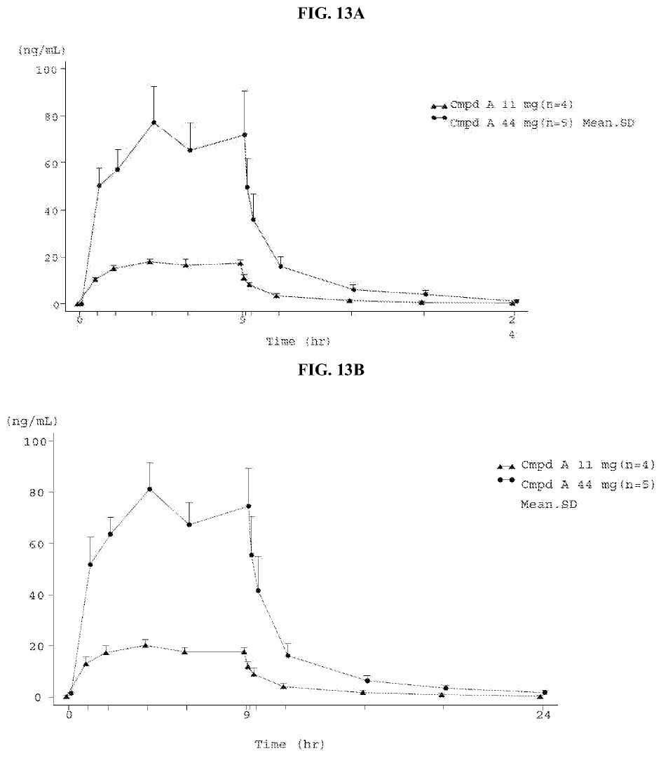

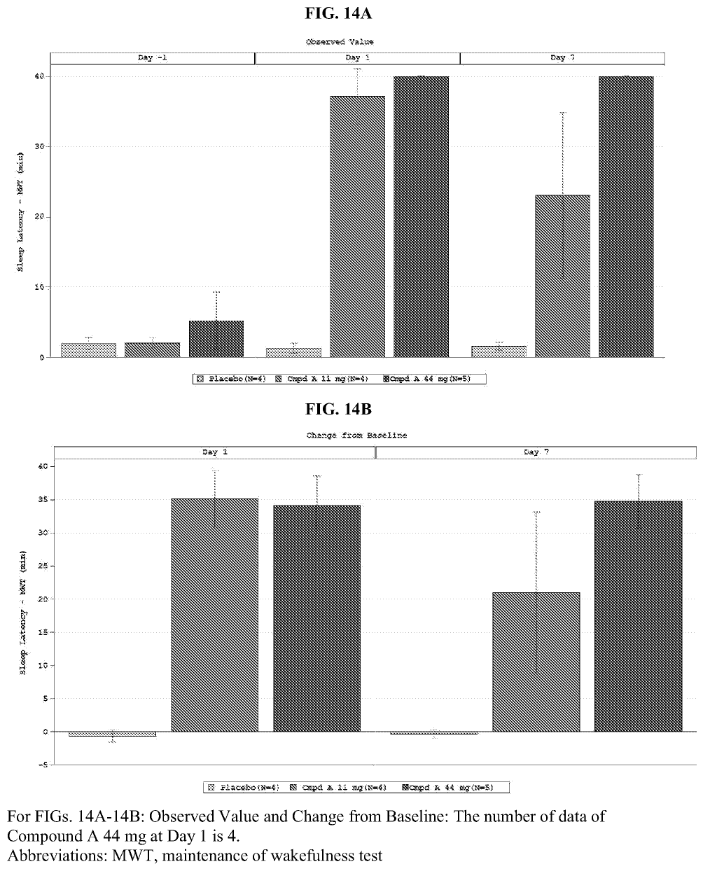

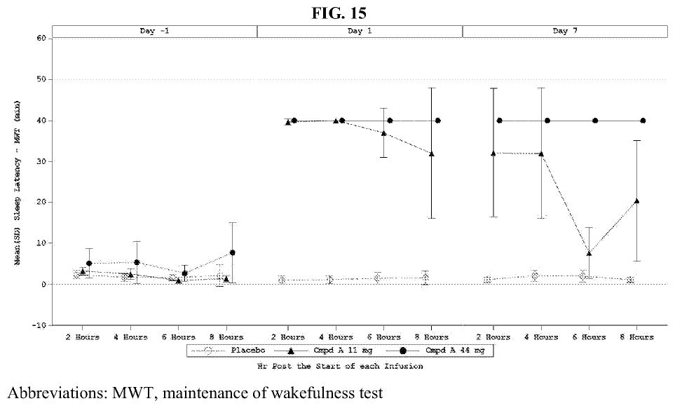

Average sleep latency in MWT and change from baseline by visit are displayed in FIGS. 14A and 14B, respectively. Mean and standard deviation plots of sleep latency in each session in MWT by visit are shown in FIG. 15.

In patients with NT1, the average sleep latency in MW T increased in the Compound A 11 mg group and Compound A 44 mg group compared with the placebo group. On Day 1, the mean sleep latency was 37.16 minutes and 40.00 minutes in the Compound A 11 mg group and Compound A 44 mg group, respectively, compared with 1.31 minutes in the placebo group. On Day 7, the mean sleep latency was 23.03 minutes and 40.00 minutes in the Compound A 11 mg group and Compound A 44 mg group, respectively, compared with 1.59 minutes in the placebo group. The mean changes from baseline in average sleep latency in MWT (min) were −0.66, 35.13, and 34.19 on Day 1 and −0.38, 21.00, and 34.78 on Day 7 for placebo, Compound A 11 mg, and Compound A 44 mg, respectively.

During 9-hour IV infusion of Compound A, a significant increase in sleep latency was observed in all active groups compared with placebo. The maximum sleep latency of 40 minutes was achieved in the Compound A 11 mg group at 4 hours on Day 1 and in the Compound A 44 mg group at all time points on both Day 1 and Day 7.

### Example II-3: ESS

Summaries of ESS and change from baseline are presented in Error! Reference source not found.24.

In patients with NT1, ESS was improved in the Compound A 11 mg and Compound A 44 mg groups compared with the placebo group. On Day 7, the mean ESS score was 9.8 and 0.0 in the Compound A 11 mg group and Compound A 44 mg group, respectively, compared with 16.3 in the placebo group. The mean changes from baseline in ESS were −0.3, −8.3, and −18.6 on Day 7 in the placebo, Compound A 11 mg, and Compound A 44 mg groups, respectively.

TABLE 24
Summary of ESS and Change from Baseline by Visit (Cohort B1, B2)

| | | Compound A | | Compound A | |
|---|---|---|---|---|---|
| Placebo | | 11 mg | | 44 mg | |
| (N = 4) | | (N = 4) | | (N = 5) | |
| | Change | | Change | | Change |
| Observed | from | Observed | from | Observed | from |
| Value | Baseline | Value | Baseline | Value | Baseline |

Baseline (Day −1)

| | | | |
|---|---|---|---|
| N | 4 | 4 | 5 |
| Mean | 16.5 | 18.0 | 18.6 |
| SD | 4.65 | 4.69 | 4.04 |
| Median | 17.5 | 17.5 | 18.0 |
| Min, max | 10, 21 | 13, 24 | 13, 23 |

Day 7

| | | | | | | |
|---|---|---|---|---|---|---|
| N | 4 | 4 | 4 | 4 | 5 | 5 |
| Mean | 16.3 | −0.3 | 9.8 | −8.3 | 0.0 | −18.6 |
| SD | 5.06 | 1.26 | 3.59 | 7.50 | 0.00 | 4.04 |
| Median | 16.5 | 0.0 | 8.5 | −9.5 | 0.0 | −18.0 |
| Min, max | 10, 22 | −2, 1 | 7, 15 | −16, 2 | 0, 0 | −23, −13 |

Abbreviation: ESS, Epworth Sleepiness Scale

### Example II-4: KSS

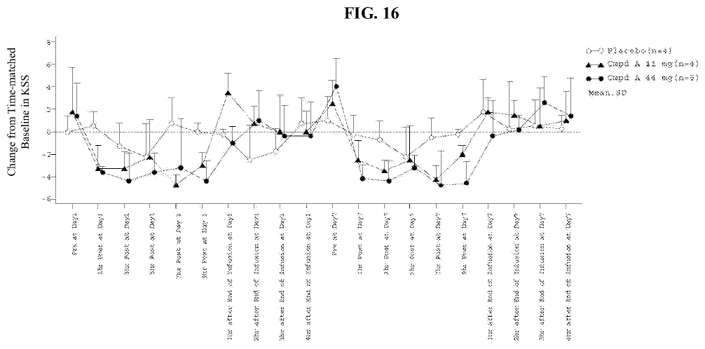

Mean and standard deviation plots of change from time-matched baseline in KSS are provided in FIG. 16.

In patients with NT1, KSS scores were generally lower (greater alertness) in the Compound A 11 mg and Compound A 44 mg groups compared with the placebo group during 9-hour IV infusion period. The mean changes from baseline in KSS at 9 hours postdose were 0.0, −3.0, and −4.4 on Day 1 and −0.3, −2.0, and −4.6 on Day 7 in the placebo, Compound A 11 mg, and Compound A 44 mg groups, respectively.

### Example II-5: PVT

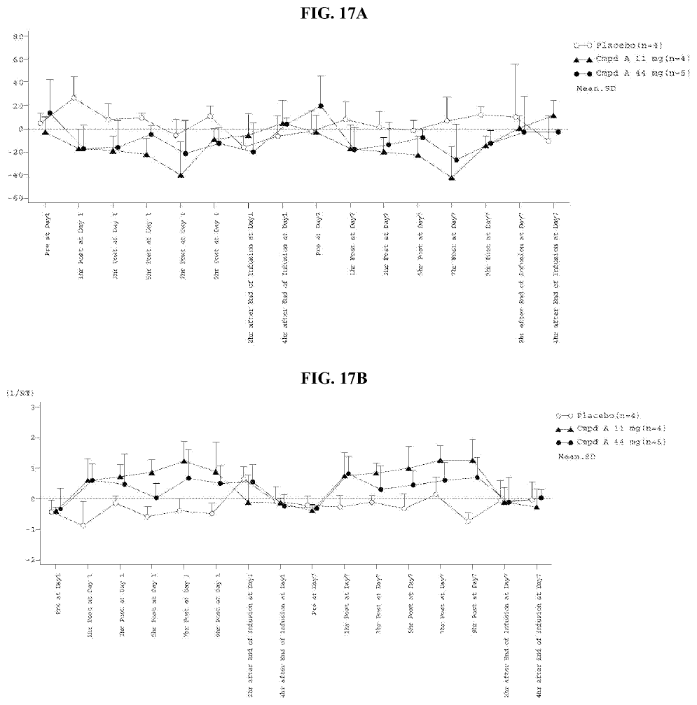

Mean and standard deviation plots of change from time-matched baseline of two PVT parameters are presented in FIGS. 17A and 17B.

In patients with NT1, PVT scores were generally improved (greater alertness) in the Compound A 11 mg and Compound A 44 mg groups compared with the placebo group during 9-hour IV infusion period. The mean changes from baseline in the number of lapses at 9 hours postdose were 10.5, −9.3, and −12.8 on Day 1 and 11.8, −14.8, and −12.8 on Day 7 in the placebo, Compound A 11 mg, and Compound A 44 mg groups, respectively. The mean changes from baseline in mean duration of the reciprocal 10% slowest reaction time (1/RT) at 9 hours postdose were −0.49, 0.88, and 0.51 on Day 1 and −0.73, 1.27, and −0.70 on Day 7 in the placebo, Compound A 11 mg, and Compound A 44 mg groups, respectively.

### Example II-6: Cataplexy

Summaries of number of cataplexy episodes by visit are presented in Table 25 and Table 26.

In patients with NT1, the mean number of cataplexy episodes during IV infusion in 7-day treatment period was reduced from 5.8 to 0 in the Compound A 11 mg group and from 3.2 to 0 in the Compound A 44 mg group compared with from 2.3 to 1.8 in the placebo group.

TABLE 25
Summary of Number of Cataplexy Episodes
by Visit (Cohort B1, B2)

| | | |
|---|---|---|
| | Cmpd A | Cmpd A |
| Placebo | 11 mg | 44 mg |
| (N = 4) | (N = 4) | (N = 5) |

Baseline (Day −7-1 (before infusion))

| | | | |
|---|---|---|---|
| N | 4 | 4 | 5 |
| Mean | 6.5 | 11.5 | 3.6 |
| SD | 5.07 | 10.50 | 2.70 |
| Median | 4.5 | 7.0 | 2.0 |
| Min, max | 3, 14 | 5, 27 | 1, 7 |

Treatment Period (Day 1-7 [until 24 hours after start of infusion])

| | | | |
|---|---|---|---|
| N | 4 | 4 | 5 |
| Mean | 2.8 | 3.0 | 1.4 |
| SD | 3.77 | 4.76 | 1.14 |
| Median | 1.5 | 1.0 | 1.0 |
| Min, max | 0, 8 | 0, 10 | 0, 3 |

TABLE 26
Summary of Number of Cataplexy Episodes
by Visit (Time-matched) (Cohort B1, B2)

| | | |
|---|---|---|
| | Cmpd A | Cmpd A |
| Placebo | 11 mg | 44 mg |
| (N = 4) | (N = 4) | (N = 5) |

Day −7-1 (time matched with during infusion time)

| | | | |
|---|---|---|---|
| N | 4 | 4 | 5 |
| Mean | 2.3 | 5.8 | 3.2 |
| SD | 1.50 | 7.68 | 2.59 |
| Median | 2.0 | 3.0 | 2.0 |
| Min, max | 1, 4 | 0, 17 | 1, 6 |

Day −7-1 (time matched with after infusion time)

| | | | |
|---|---|---|---|
| N | 4 | 4 | 5 |
| Mean | 4.3 | 5.8 | 0.4 |
| SD | 5.85 | 2.99 | 0.55 |
| Median | 1.5 | 5.0 | 0.0 |
| Min, max | 1, 13 | 3, 10 | 0, 1 |

Day 1-7 (during infusion time)

| | | | |
|---|---|---|---|
| N | 4 | 4 | 5 |
| Mean | 1.8 | 0.0 | 0.0 |
| SD | 2.06 | 0.00 | 0.00 |
| Median | 1.5 | 0.0 | 0.0 |
| Min, max | 0, 4 | 0, 0 | 0, 0 |

Day 1-7 (after infusion time up to 24 hours after start of infusion)

| | | | |
|---|---|---|---|
| N | 4 | 4 | 5 |
| Mean | 1.0 | 3.0 | 1.4 |
| SD | 2.00 | 4.76 | 1.14 |
| Median | 0.0 | 1.0 | 1.0 |
| Min, max | 0, 4 | 0, 10 | 0, 3 |

### CONCLUSIONS

Compound A was safe and well tolerated in the multiple IV administrations of up to 44 mg for patients with NT1. There was no SAE, and no TEAE leading to study drug discontinuation. Dose dependent increase in frequency of TEAEs and drug-related TEAEs was generally observed.

A trend for the observed BP and PR increases with increasing Compound A plasma exposure was observed in NT1 patient populations.

Following daily 9-hour IV infusions, mean plasma systemic exposures of Compound A, which were similar between healthy adults and the patients with NT1, generally increased in a dose proportional manner over the dose range studied. Consistent with the short t_{1/2z}, no drug accumulation was observed with QD dosing of 9-hour IV infusions.

In patients with NT1, the mean sleep latency of MWT (min) on Day 7 was 40.00 and 23.03 in the Compound A 44 mg group and the Compound A 11 mg group, respectively, compared with 1.59 in the placebo group. The maximum sleep latency as 40 minutes of wakefulness was achieved in the Compound A 11 mg group at 4 hours on Day 1 and in the Compound A 44 mg group at all time points on both Day 1 and Day 7.

The results of ESS/KSS, PGI-C, PVT and number of naps during the day generally supported the wakefulness PD effect observed in MWT.

In patients with NT1, the mean number of cataplexy episodes during treatment period was decreased from baseline and no cataplexy episodes were reported in the Compound A 11 mg group and Compound A 44 mg group (0 in both groups), while episodes remained in the placebo group (1.8) during IV infusion.

TABLE 18
List of Abbreviations

| Term | Definition |
|---|---|
| AE | adverse event |
| ALT | alanine aminotransferase |
| AST | aspartate aminotransferase |
| AUC | area under the concentration-time curve |
| AUCₗₐₛₜ | Area under the concentration-time curve from time 0 to time of the last |
| | quantifiable concentration |
| AUC_{∞} (or AUC_{inf}) | area under the concentration-time curve from time 0 to infinity |
| AUC_{τ} | Area under the plasma concentration-time curve during a dosing interval |
| BMI | body mass index |
| BP | blood pressure |
| BSES | betadex sulfobutyl ether sodium |
| Cₑₒᵢ | concentration at end of infusion |
| Cₘₐₓ | maximum observed concentration |
| Cₘᵢₙ | Minimum observed concentration during a dosing interval |
| CNS | central nervous system |
| CRA | clinical research associate |
| CRP | c-reactive protein |
| CSF | cerebrospinal fluid |
| C-SSRS | Columbia Suicide Severity Rating Scale |
| DBP | diastolic blood pressure |
| DNA | deoxyribonucleic acid |
| ECG | electrocardiogram |
| eCRF | electronic case report form |
| ESS | Epworth Sleepiness Scale |
| FDA | U.S. Food and Drug Administration |
| FIH | first-in-human |
| GCP | Good Clinical Practice |
| γ-GTP | gamma-glutamyl transpeptidase |
| HBsAg | hepatitis B virus surface antigen |
| HCV | hepatitis C virus |
| HED | human equivalence dose |
| HIV | human immunodeficiency virus |
| HLA | human leukocyte antigen |
| ICH | International Conference on Harmonization of Technical Requirements |
| | for Registration of Pharmaceuticals for Human Use |
| ICSD-3 | International Classification of Sleep Disorders, third edition |
| IEC | independent ethics committee |
| INR | international normalized ratio |
| IRB | Institutional Review Board |
| IV | intravenous |
| KO | knock out |
| KSS | Karolinska Sleepiness Scale |
| LFT | liver function test |
| MedDRA | Medical Dictionary for Regulatory Activities |
| MHRA | The Medicines and Healthcare Products Regulatory Agency |
| MR | Metabolic Ratio |
| MRSD | maximum recommended starting dose |
| MSLT | multiple sleep latency test |
| MWT | maintenance of wakefulness test |
| NOAEL | no observed adverse effect level |
| NT1 | narcolepsy type 1 |
| OX | orexin |
| OX2R | orexin 2 receptor |
| OTC | over-the-counter |
| PD | pharmacodynamics |
| PGx | pharmacogenomics |
| PK | pharmacokinetics |
| PT | Preferred Term |
| PMDA | The Pharmaceuticals and Medical Devices Agency |
| PTR | Peak trough ratio during a dosing interval, at steady state |
| POMS | profile of mood states |
| PSG | polysomnography |
| QTcF | corrected QT with Fredericia correction method |
| R_{ac(AUC)} | Accumulation ratio based on AUC_{τ} |
| R_{ac(Cmax)} | Accumulation ratio based on Cmax |
| RBC | red blood cell |
| REM | rapid eye movement |
| SAE | serious adverse event |
| SAP | statistical analysis plan |
| SBP | systolic blood pressure |
| SOREM | sleep-onset rapid eye movement |
| SSRIs | selective serotonin reuptake inhibitors |
| SUSAR | suspected unexpected serious adverse reactions |
| TEAE | treatment-emergent adverse event |
| tₘₐₓ | Time of first occurrence of Cmax |
| t_{1/2z} | Terminal disposition phase half-life |
| Vₛₛ | Volume of distribution at steady-state after intravenous administration (only |
| | for Compound A) |
| V_{z} | Volume of distribution during the terminal phase after intravenous |
| | administration (only for Compound A) |
| CL | Total clearance after intravenous administration (only for Compound A) |
| ULN | upper limit of normal |
| WBC | white blood cell |
| WT | wild type |

TABLE 5
Summary of Plasma Concentrations by Visit (Cohort 1, 2)
<Pharmacokinetic Analysis Set>

| | | Visit | | | | | | | | | | |
|---|---|---|---|---|---|---|---|---|---|---|---|---|
| | | | | | | | | | | | | 0.17 hr |
| | | | | | | | | | | | | After |
| Analyte/ | | | | | | | | | | | | End of |
| Treatment | Statistics | Predose | 0.5 hr | 1 hr | 1.5 hr | 2 hr | 3 hr | 4 hr | 6 hr | 8 hr | 9 hr | Infusion |
| Cmpd A (ng/mL) | | | | | | | | | | | | |
| Cmpd A 7 mg | N | 6 | 6 | 6 | 6 | 6 | 6 | 6 | 5 | 5 | 5 | 5 |
| | Mean | 0.000 | 9.148 | 11.72 | 13.09 | 14.75 | 16.48 | 15.92 | 12.20 | 12.98 | 13.50 | 9.360 |
| | SD | 0.0000 | 1.4989 | 2.3766 | 2.8196 | 2.7812 | 2.5740 | 3.1276 | 2.5438 | 2.3488 | 2.0174 | 0.98329 |
| | Minimum | 0.00 | 6.99 | 8.68 | 8.96 | 10.9 | 13.2 | 11.1 | 8.82 | 9.50 | 10.2 | 7.91 |
| | Q1 | 0.000 | 7.830 | 9.450 | 12.00 | 12.80 | 15.10 | 15.20 | 11.40 | 12.80 | 13.20 | 9.090 |
| | Median | 0.000 | 9.535 | 11.95 | 13.00 | 14.90 | 15.80 | 15.50 | 12.40 | 13.10 | 13.90 | 9.460 |
| | Q3 | 0.000 | 10.50 | 13.20 | 14.00 | 16.50 | 18.80 | 17.60 | 12.50 | 13.40 | 15.00 | 9.740 |
| | Maximum | 0.00 | 10.5 | 15.1 | 17.6 | 18.5 | 20.2 | 20.6 | 15.9 | 16.1 | 15.2 | 10.6 |
| Cmpd A 14 mg | N | 6 | 6 | 6 | 6 | 6 | 6 | 6 | 6 | 6 | 6 | 6 |
| | Mean | 0.000 | 22.50 | 26.65 | 30.45 | 32.88 | 35.83 | 36.40 | 29.33 | 28.95 | 30.08 | 19.38 |
| | SD | 0.0000 | 3.0113 | 3.9753 | 3.6566 | 3.2646 | 4.6072 | 3.8163 | 5.3339 | 4.5161 | 4.2579 | 3.1403 |
| | Minimum | 0.00 | 18.0 | 21.8 | 27.4 | 29.6 | 31.8 | 31.0 | 23.1 | 22.6 | 22.8 | 14.1 |
| | Q1 | 0.000 | 21.70 | 22.60 | 27.60 | 30.00 | 32.10 | 33.00 | 23.90 | 24.60 | 28.40 | 18.00 |
| | Median | 0.000 | 22.10 | 26.85 | 29.00 | 32.05 | 34.30 | 37.00 | 29.70 | 29.90 | 30.80 | 19.65 |
| | Q3 | 0.000 | 23.90 | 29.70 | 33.60 | 35.80 | 39.00 | 40.00 | 34.40 | 32.30 | 33.50 | 22.20 |
| | Maximum | 0.00 | 27.2 | 32.1 | 36.1 | 37.8 | 43.5 | 40.4 | 35.2 | 34.4 | 34.2 | 22.7 |

| | | Visit | | | | |
|---|---|---|---|---|---|---|
| | | 0.33 hr | 0.5 hr | 0.75 hr | 1 hr | 1.5 hr |
| | | After | After | After | After | After |
| Analyte/ | | End of | End of | End of | End of | End of |
| Treatment | Statistics | Infusion | Infusion | Infusion | Infusion | Infusions |
| Cmpd A (ng/mL) | | | | | | |
| Cmpd A 7 mg | N | 5 | 5 | 5 | 5 | 5 |
| | Mean | 7.336 | 6.728 | 5.548 | 4.908 | 4.250 |
| | SD | 0.93823 | 0.80120 | 0.30203 | 0.47092 | 0.58919 |
| | Minimum | 6.28 | 6.10 | 5.27 | 4.34 | 3.27 |
| | Q1 | 7.120 | 6.260 | 5.350 | 4.820 | 4.150 |
| | Median | 7.160 | 6.460 | 5.380 | 4.830 | 4.500 |
| | Q3 | 7.260 | 6.720 | 5.790 | 4.900 | 4.590 |
| | Maximum | 8.86 | 8.10 | 5.95 | 5.65 | 4.74 |
| Cmpd A 14 mg | N | 6 | 6 | 6 | 6 | 6 |
| | Mean | 16.58 | 14.95 | 12.78 | 11.65 | 10.22 |
| | SD | 3.1530 | 2.9453 | 2.5324 | 2.4444 | 2.3670 |
| | Minimum | 11.5 | 10.6 | 9.06 | 8.00 | 6.95 |
| | Q1 | 14.90 | 12.80 | 10.90 | 10.80 | 8.660 |
| | Median | 17.00 | 15.15 | 12.90 | 11.15 | 10.12 |
| | Q3 | 18.60 | 17.90 | 15.00 | 14.20 | 12.00 |
| | Maximum | 20.5 | 18.1 | 15.9 | 14.6 | 13.5 |

| | | Visit | | | | |
|---|---|---|---|---|---|---|
| | | 2 hr | 3 hr | 4 hr | 6 hr | 15 hr |
| | | After | After | After | After | After |
| Analyte/ | | End of | End of | End of | End of | End of |
| Treatment | Statistics | Infusions | Infusions | Infusions | Infusions | Infusions |
| Cmpd A (ng/mL) | | | | | | |
| Cmpd A 7 mg | N | 5 | 5 | 5 | 5 | 5 |
| | Mean | 2.516 | 1.774 | 1.490 | 0.9622 | 0.1878 |
| | SD | 0.31246 | 0.23255 | 0.33272 | 0.21131 | 0.25812 |
| | Minimum | 2.26 | 1.45 | 1.06 | 0.746 | 0.00 |
| | Q1 | 2.290 | 1.610 | 1.280 | 0.7990 | 0.000 |
| | Median | 2.340 | 1.890 | 1.480 | 0.9060 | 0.000 |
| | Q3 | 2.730 | 1.930 | 1.780 | 1.120 | 0.4380 |
| | Maximum | 2.96 | 1.99 | 1.85 | 1.24 | 0.501 |
| Cmpd A 14 mg | N | 6 | 6 | 6 | 6 | 6 |
| | Mean | 6.888 | 4.567 | 3.873 | 2.355 | 0.7192 |
| | SD | 1.7499 | 0.96757 | 0.98881 | 0.81006 | 0.58301 |
| | Minimum | 4.84 | 3.41 | 2.97 | 1.56 | 0.237 |
| | Q1 | 5.060 | 3.890 | 3.100 | 1.700 | 0.3360 |
| | Median | 7.110 | 4.435 | 3.465 | 2.090 | 0.4820 |
| | Q3 | 8.060 | 5.120 | 5.000 | 3.300 | 0.9980 |
| | Maximum | 9.15 | 6.11 | 5.24 | 3.39 | 1.78 |

| Analyte/ | | Visit | | | | | |
|---|---|---|---|---|---|---|---|
| Treatment | Statistics | Predose | 0.5 hr | 1 hr | 1.5 hr | 2 hr | 3 hr |
| Cmpd A (ng/mL) | | | | | | | |
| Cmpd A 28 mg | N | 5 | 5 | 5 | 5 | 5 | 5 |
| | Mean | 0.000 | 34.40 | 37.88 | 44.90 | 50.06 | 55.02 |
| | SD | 0.0000 | 11.561 | 10.992 | 10.775 | 10.504 | 12.127 |
| | Minimum | 0.00 | 19.1 | 21.7 | 27.2 | 33.7 | 35.6 |
| | Q1 | 0.000 | 29.60 | 36.50 | 44.40 | 45.50 | 51.30 |
| | Median | 0.000 | 32.00 | 36.60 | 47.10 | 54.90 | 59.30 |
| | Q3 | 0.000 | 42.60 | 42.80 | 49.90 | 57.80 | 63.70 |
| | Maximum | 0.00 | 48.7 | 51.8 | 55.9 | 58.4 | 65.2 |
| Cmpd A 56 mg | N | 6 | 6 | 6 | 6 | 6 | 6 |
| | Mean | 0.000 | 71.17 | 81.15 | 92.78 | 99.53 | 114.2 |
| | SD | 0.0000 | 6.2121 | 6.6377 | 13.255 | 12.118 | 16.146 |
| | Minimum | 0.00 | 64.4 | 74.8 | 75.5 | 86.5 | 98.3 |
| | Q1 | 0.000 | 65.00 | 76.20 | 88.80 | 88.80 | 98.60 |
| | Median | 0.000 | 70.70 | 79.05 | 90.15 | 98.95 | 112.0 |
| | Q3 | 0.000 | 75.90 | 86.30 | 96.10 | 110.0 | 131.0 |
| | Maximum | 0.00 | 80.3 | 91.5 | 116 | 114 | 133 |
| Cmpd A 112 mg | N | 6 | 6 | 6 | 6 | 6 | 6 |
| | Mean | 0.000 | 132.2 | 153.8 | 194.5 | 200.3 | 222.5 |
| | SD | 0.0000 | 24.588 | 20.034 | 40.639 | 26.681 | 35.971 |
| | Minimum | 0.00 | 108 | 126 | 141 | 150 | 171 |
| | Q1 | 0.000 | 119.0 | 131.0 | 164.0 | 195.0 | 209.0 |
| | Median | 0.000 | 121.5 | 162.5 | 197.0 | 207.0 | 215.5 |
| | Q3 | 0.000 | 149.0 | 169.0 | 213.0 | 220.0 | 248.0 |
| | Maximum | 0.00 | 174 | 172 | 255 | 223 | 276 |
| Cmpd A 134.4 mg | N | 6 | 6 | 6 | 6 | 6 | 6 |
| | Mean | 0.000 | 161.7 | 204.2 | 228.2 | 248.2 | 287.8 |
| | SD | 0.0000 | 20.897 | 36.169 | 36.967 | 56.198 | 57.339 |
| | Minimum | 0.00 | 147 | 181 | 191 | 192 | 217 |
| | Q1 | 0.000 | 148.0 | 181.0 | 197.0 | 199.0 | 260.0 |
| | Median | 0.000 | 152.5 | 187.0 | 221.0 | 243.5 | 278.5 |
| | Q3 | 0.000 | 169.0 | 216.0 | 250.0 | 276.0 | 305.0 |
| | Maximum | 0.00 | 201 | 273 | 289 | 335 | 388 |

| | | Visit | | | | |
|---|---|---|---|---|---|---|
| | | | | | | 0.17 hr |
| | | | | | | After |
| Analyte/ | | | | | | End of |
| Treatment | Statistics | 4 hr | 6 hr | 8 hr | 9 hr | Infusion |
| Cmpd A (ng/mL) | | | | | | |
| Cmpd A 28 mg | N | 5 | 5 | 5 | 5 | 5 |
| | Mean | 55.00 | 46.82 | 48.68 | 52.52 | 32.52 |
| | SD | 6.9516 | 4.3292 | 8.5025 | 7.2344 | 2.5801 |
| | Minimum | 44.4 | 40.2 | 37.1 | 40.6 | 28.2 |
| | Q1 | 54.20 | 45.40 | 43.30 | 51.00 | 32.70 |
| | Median | 54.50 | 48.00 | 51.70 | 55.30 | 32.80 |
| | Q3 | 58.80 | 48.80 | 52.50 | 57.40 | 34.00 |
| | Maximum | 63.1 | 51.7 | 58.8 | 58.3 | 34.9 |
| Cmpd A 56 mg | N | 6 | 6 | 6 | 6 | 6 |
| | Mean | 117.7 | 91.82 | 102.1 | 115.2 | 63.58 |
| | SD | 13.663 | 17.920 | 17.648 | 13.303 | 9.2569 |
| | Minimum | 107 | 73.5 | 84.9 | 102 | 54.5 |
| | Q1 | 109.0 | 73.80 | 87.20 | 104.0 | 55.60 |
| | Median | 113.0 | 90.80 | 99.65 | 112.5 | 62.50 |
| | Q3 | 120.0 | 103.0 | 108.0 | 122.0 | 66.50 |
| | Maximum | 144 | 119 | 133 | 138 | 79.9 |
| Cmpd A 112 mg | N | 6 | 6 | 6 | 6 | 6 |
| | Mean | 224.0 | 189.2 | 194.2 | 215.8 | 126.2 |
| | SD | 45.242 | 19.052 | 22.675 | 31.714 | 14.331 |
| | Minimum | 154 | 154 | 162 | 169 | 106 |
| | Q1 | 215.0 | 186.0 | 176.0 | 192.0 | 110.0 |
| | Median | 216.5 | 192.0 | 195.0 | 221.0 | 133.0 |
| | Q3 | 252.0 | 205.0 | 216.0 | 245.0 | 137.0 |
| | Maximum | 290 | 206 | 221 | 247 | 138 |
| Cmpd A 134.4 mg | N | 6 | 6 | 6 | 6 | 6 |
| | Mean | 294.0 | 247.8 | 261.7 | 291.8 | 181.7 |
| | SD | 51.088 | 55.470 | 41.220 | 32.096 | 43.173 |
| | Minimum | 214 | 164 | 199 | 241 | 145 |
| | Q1 | 256.0 | 206.0 | 242.0 | 268.0 | 148.0 |
| | Median | 307.5 | 260.0 | 262.5 | 300.5 | 167.5 |
| | Q3 | 324.0 | 295.0 | 291.0 | 311.0 | 207.0 |
| | Maximum | 355 | 302 | 313 | 330 | 255 |

| | | Visit | | | | | |
|---|---|---|---|---|---|---|---|
| | | 0.33 hr | 0.5 hr | 0.75 hr | 1 hr | 1.5 hr | 2 hr |
| | | After | After | After | After | After | After |
| Analyte/ | | End of | End of | End of | End of | End of | End of |
| Treatment | Statistics | Infusion | Infusion | Infusion | Infusion | Infusions | Infusions |
| Cmpd A (ng/mL) | | | | | | | |
| Cmpd A 28 mg | N | 5 | 5 | 5 | 5 | 5 | 5 |
| | Mean | 28.04 | 25.36 | 20.82 | 18.56 | 16.70 | 10.53 |
| | SD | 2.6083 | 5.2219 | 2.2731 | 2.2244 | 2.3098 | 2.3377 |
| | Minimum | 25.4 | 20.9 | 19.0 | 15.6 | 13.6 | 8.06 |
| | Q1 | 26.70 | 21.30 | 19.10 | 17.80 | 14.90 | 8.990 |
| | Median | 27.20 | 24.20 | 19.50 | 18.00 | 18.00 | 9.600 |
| | Q3 | 28.70 | 26.70 | 22.60 | 20.00 | 18.10 | 12.60 |
| | Maximum | 32.2 | 33.7 | 23.9 | 21.4 | 18.9 | 13.4 |
| Cmpd A 56 mg | N | 6 | 6 | 6 | 6 | 6 | 6 |
| | Mean | 54.37 | 50.33 | 45.77 | 41.83 | 33.63 | 21.70 |
| | SD | 10.779 | 11.101 | 9.5728 | 9.9478 | 7.8727 | 7.1420 |
| | Minimum | 44.7 | 38.4 | 33.3 | 30.1 | 27.4 | 15.4 |
| | Q1 | 45.00 | 40.90 | 36.90 | 34.80 | 28.40 | 15.60 |
| | Median | 53.25 | 50.10 | 47.20 | 39.80 | 31.35 | 20.20 |
| | Q3 | 56.20 | 53.10 | 50.40 | 49.50 | 35.00 | 24.20 |
| | Maximum | 73.8 | 69.4 | 59.6 | 57.0 | 48.3 | 34.6 |
| Cmpd A 112 mg | N | 6 | 6 | 6 | 6 | 6 | 6 |
| | Mean | 104.7 | 95.03 | 81.02 | 73.08 | 58.00 | 42.08 |
| | SD | 14.222 | 12.286 | 9.9903 | 9.9062 | 8.7130 | 9.2892 |
| | Minimum | 87.1 | 78.0 | 66.3 | 62.8 | 44.9 | 31.4 |
| | Q1 | 92.10 | 86.50 | 72.40 | 64.10 | 51.50 | 33.00 |
| | Median | 105.5 | 94.85 | 83.95 | 71.55 | 59.00 | 42.50 |
| | Q3 | 111.0 | 103.0 | 85.40 | 79.30 | 64.70 | 49.20 |
| | Maximum | 127 | 113 | 94.1 | 89.2 | 68.9 | 53.9 |
| Cmpd A 134.4 mg | N | 6 | 6 | 6 | 6 | 6 | 6 |
| | Mean | 140.5 | 123.2 | 108.7 | 92.57 | 81.10 | 56.18 |
| | SD | 28.787 | 18.809 | 21.620 | 15.738 | 19.182 | 14.654 |
| | Minimum | 105 | 101 | 82.4 | 75.2 | 65.4 | 42.1 |
| | Q1 | 122.0 | 102.0 | 83.70 | 78.90 | 66.20 | 44.70 |
| | Median | 138.0 | 127.0 | 112.5 | 90.65 | 75.80 | 52.20 |
| | Q3 | 157.0 | 132.0 | 126.0 | 102.0 | 88.40 | 64.40 |
| | Maximum | 183 | 150 | 135 | 118 | 115 | 81.5 |

| | | Visit | | | | |
|---|---|---|---|---|---|---|
| | | 3 hr | 4 hr | 6 hr | 10 hr | 15 hr |
| | | After | After | After | After | After |
| Analyte/ | | End of | End of | End of | End of | End of |
| Treatment | Statistics | Infusions | Infusions | Infusions | Infusions | Infusions |
| Cmpd A (ng/mL) | | | | | | |
| Cmpd A 28 mg | N | 5 | 5 | 5 | 5 | 5 |
| | Mean | 8.746 | 7.458 | 3.948 | 2.510 | 1.241 |
| | SD | 2.9770 | 2.1776 | 1.4345 | 0.88213 | 0.81253 |
| | Minimum | 5.23 | 5.09 | 2.34 | 1.67 | 0.402 |
| | Q1 | 7.640 | 6.020 | 3.040 | 1.810 | 0.8820 |
| | Median | 8.410 | 7.600 | 3.510 | 2.440 | 1.120 |
| | Q3 | 9.050 | 7.780 | 5.060 | 2.760 | 1.220 |
| | Maximum | 13.4 | 10.8 | 5.79 | 3.87 | 2.58 |
| Cmpd A 56 mg | N | 6 | 6 | 6 | 6 | 6 |
| | Mean | 18.22 | 14.62 | 8.382 | 4.343 | 1.885 |
| | SD | 5.7343 | 5.2147 | 4.1282 | 3.3217 | 1.5979 |
| | Minimum | 10.9 | 8.12 | 4.44 | 2.21 | 0.767 |
| | Q1 | 13.90 | 11.20 | 6.650 | 2.320 | 0.8930 |
| | Median | 18.30 | 14.60 | 7.300 | 3.370 | 1.510 |
| | Q3 | 20.40 | 15.70 | 8.200 | 3.790 | 1.560 |
| | Maximum | 27.5 | 23.5 | 16.4 | 11.0 | 5.07 |
| Cmpd A 112 mg | N | 6 | 6 | 6 | 6 | 6 |
| | Mean | 38.13 | 30.68 | 17.42 | 8.967 | 3.897 |
| | SD | 12.634 | 11.927 | 7.5307 | 5.0200 | 2.9704 |
| | Minimum | 19.7 | 17.4 | 9.58 | 3.40 | 1.30 |
| | Q1 | 30.00 | 19.70 | 9.840 | 4.130 | 1.610 |
| | Median | 39.25 | 30.45 | 17.25 | 9.435 | 3.055 |
| | Q3 | 47.30 | 37.80 | 21.60 | 10.10 | 5.040 |
| | Maximum | 53.3 | 48.3 | 29.0 | 17.3 | 9.32 |
| Cmpd A 134.4 mg | N | 6 | 6 | 6 | 6 | 6 |
| | Mean | 41.53 | 33.52 | 19.28 | 10.45 | 4.465 |
| | SD | 14.123 | 10.079 | 7.0365 | 5.9609 | 3.3768 |
| | Minimum | 22.2 | 18.5 | 9.56 | 5.43 | 1.54 |
| | Q1 | 32.50 | 27.80 | 16.10 | 6.380 | 2.300 |
| | Median | 40.60 | 33.20 | 18.25 | 9.030 | 3.635 |
| | Q3 | 51.30 | 41.70 | 23.00 | 11.00 | 4.780 |
| | Maximum | 62.0 | 46.7 | 30.5 | 21.8 | 10.9 |

Visit

| | | | | | | | | | | | | 0.17 hr |
|---|---|---|---|---|---|---|---|---|---|---|---|---|
| | | | | | | | | | | | | After |
| Analyte/ | | | | | | | | | | | | End of |
| Treatment | Statistics | Predose | 0.5 hr | 1 hr | 1.5 hr | 2 hr | 3 hr | 4 hr | 6 hr | 8 hr | 9 hr | Infusion |
| Cmpd A (ng/mL) | | | | | | | | | | | | |
| Cmpd A 180 mg | N | 6 | 6 | 6 | 6 | 6 | 6 | 6 | 6 | 6 | 6 | 6 |
| | Mean | 0.000 | 180.5 | 216.3 | 254.8 | 294.5 | 315.0 | 327.7 | 292.2 | 300.3 | 325.2 | 195.0 |
| | SD | 0.0000 | 24.386 | 31.411 | 34.132 | 34.198 | 25.597 | 29.615 | 24.145 | 27.023 | 30.182 | 32.062 |
| | Minimum | 0.00 | 161 | 173 | 224 | 251 | 276 | 293 | 252 | 275 | 285 | 154 |
| | Q1 | 0.000 | 163.0 | 201.0 | 228.0 | 271.0 | 307.0 | 304.0 | 285.0 | 277.0 | 311.0 | 159.0 |
| | Median | 0.000 | 170.5 | 215.0 | 241.5 | 292.0 | 314.0 | 325.0 | 291.5 | 291.5 | 316.5 | 205.0 |
| | Q3 | 0.000 | 194.0 | 229.0 | 290.0 | 320.0 | 324.0 | 358.0 | 311.0 | 330.0 | 358.0 | 223.0 |
| | Maximum | 0.00 | 224 | 265 | 304 | 341 | 355 | 361 | 322 | 337 | 364 | 224 |
| Cmpd A 240 mg | N | 6 | 6 | 6 | 6 | 6 | 6 | 6 | 6 | 6 | 6 | 6 |
| | Mean | 0.000 | 292.2 | 345.0 | 388.3 | 403.0 | 455.5 | 493.0 | 427.7 | 474.5 | 497.2 | 328.2 |
| | SD | 0.0000 | 73.850 | 79.473 | 70.045 | 58.014 | 76.751 | 100.33 | 79.510 | 70.939 | 70.335 | 44.897 |
| | Minimum | 0.00 | 228 | 265 | 315 | 337 | 393 | 388 | 335 | 393 | 417 | 277 |
| | Q1 | 0.000 | 230.0 | 286.0 | 333.0 | 349.0 | 400.0 | 423.0 | 366.0 | 409.0 | 452.0 | 281.0 |
| | Median | 0.000 | 263.5 | 318.0 | 369.5 | 399.0 | 421.5 | 459.0 | 409.5 | 470.5 | 481.0 | 329.5 |
| | Q3 | 0.000 | 368.0 | 418.0 | 463.0 | 452.0 | 515.0 | 579.0 | 514.0 | 542.0 | 539.0 | 360.0 |
| | Maximum | 0.00 | 400 | 465 | 480 | 482 | 582 | 650 | 532 | 562 | 613 | 392 |

| | | Visit | | | | | |
|---|---|---|---|---|---|---|---|
| | | 0.33 hr | 0.5 hr | 0.75 hr | 1 hr | 1.5 hr | 2 hr |
| | | After | After | After | After | After | After |
| Analyte/ | | End of | End of | End of | End of | End of | End of |
| Treatment | Statistics | Infusion | Infusion | Infusion | Infusion | Infusions | Infusions |
| Cmpd A (ng/mL) | | | | | | | |
| Cmpd A 180 mg | N | 6 | 6 | 6 | 6 | 6 | 6 |
| | Mean | 161.2 | 138.8 | 124.7 | 105.7 | 87.53 | 61.40 |
| | SD | 21.904 | 20.430 | 11.860 | 12.690 | 12.251 | 14.379 |
| | Minimum | 141 | 117 | 101 | 89.8 | 69.8 | 41.2 |
| | Q1 | 144.0 | 120.0 | 127.0 | 90.60 | 76.60 | 53.10 |
| | Median | 157.5 | 137.5 | 128.0 | 109.5 | 89.55 | 59.80 |
| | Q3 | 166.0 | 148.0 | 130.0 | 115.0 | 98.70 | 72.30 |
| | Maximum | 201 | 173 | 134 | 120 | 101 | 82.2 |
| Cmpd A 240 mg | N | 6 | 6 | 6 | 6 | 6 | 6 |
| | Mean | 256.3 | 220.3 | 193.7 | 172.3 | 144.7 | 97.98 |
| | SD | 40.068 | 22.722 | 22.862 | 25.500 | 16.354 | 11.552 |
| | Minimum | 200 | 182 | 148 | 127 | 113 | 77.5 |
| | Q1 | 243.0 | 215.0 | 199.0 | 168.0 | 145.0 | 94.40 |
| | Median | 249.0 | 222.5 | 200.5 | 174.5 | 148.5 | 100.5 |
| | Q3 | 276.0 | 228.0 | 202.0 | 187.0 | 154.0 | 103.0 |
| | Maximum | 321 | 252 | 212 | 203 | 159 | 112 |

| | | Visit | | | | |
|---|---|---|---|---|---|---|
| | | 3 hr | 4 hr | 6 hr | 10 hr | 15 hr |
| | | After | After | After | After | After |
| Analyte/ | | End of | End of | End of | End of | End of |
| Treatment | Statistics | Infusions | Infusions | Infusions | Infusions | Infusions |
| Cmpd A (ng/mL) | | | | | | |
| Cmpd A 180 mg | N | 6 | 6 | 6 | 6 | 6 |
| | Mean | 44.02 | 32.82 | 15.28 | 7.692 | 3.298 |
| | SD | 7.9121 | 8.5523 | 3.2860 | 4.0331 | 1.6227 |
| | Minimum | 30.3 | 22.2 | 11.7 | 3.97 | 1.85 |
| | Q1 | 40.30 | 28.20 | 12.00 | 4.910 | 2.030 |
| | Median | 45.90 | 30.10 | 15.05 | 5.985 | 2.665 |
| | Q3 | 49.20 | 42.00 | 17.70 | 11.50 | 4.760 |
| | Maximum | 52.5 | 44.3 | 20.2 | 13.8 | 5.82 |
| Cmpd A 240 mg | N | 6 | 6 | 6 | 6 | 6 |
| | Mean | 77.10 | 55.97 | 29.47 | 14.42 | 6.740 |
| | SD | 10.890 | 8.2974 | 7.3527 | 4.6449 | 2.7712 |
| | Minimum | 64.7 | 41.9 | 21.8 | 7.04 | 3.23 |
| | Q1 | 68.80 | 53.10 | 23.00 | 10.30 | 3.390 |
| | Median | 75.50 | 56.50 | 28.45 | 16.55 | 7.645 |
| | Q3 | 87.10 | 62.70 | 36.90 | 17.50 | 8.820 |
| | Maximum | 91.0 | 65.1 | 38.2 | 18.6 | 9.71 |

TABLE 6
Summary of Pharmacokinetic Parameters of Cmpd A (Cohort 1, 2)
<Pharmacokinetic Analysis Set>

| | | Statistics | | | | | |
|---|---|---|---|---|---|---|---|
| | | | | N of Geo | | | |
| Variable | Treatment | N | Mean | Mean | Geo Mean | SD | CV |
| AUClast (h*ng/mL) | Cmpd A 7 mg | 5 | 139.6 | 5 | 138.0 | 22.546 | 16.2 |
| | Cmpd A 14 mg | 6 | 325.8 | 6 | 323.1 | 45.793 | 14.1 |
| | Cmpd A 28 mg | 5 | 518.8 | 5 | 513.3 | 81.392 | 15.7 |
| | Cmpd A 56 mg | 6 | 1067 | 6 | 1055 | 178.49 | 16.7 |
| | Cmpd A 112 mg | 6 | 2088 | 6 | 2069 | 298.89 | 14.3 |
| | Cmpd A 134.4 mg | 6 | 2693 | 6 | 2655 | 495.32 | 18.4 |
| AUCinf (h*ng/mL) | Cmpd A 7 mg | 5 | 143.4 | 5 | 141.8 | 22.744 | 15.9 |
| | Cmpd A 14 mg | 6 | 331.3 | 6 | 328.1 | 51.138 | 15.4 |
| | Cmpd A 28 mg | 5 | 529.8 | 5 | 523.5 | 88.288 | 16.7 |
| | Cmpd A 56 mg | 6 | 1078 | 6 | 1065 | 187.70 | 17.4 |
| | Cmpd A 112 mg | 6 | 2113 | 6 | 2092 | 319.85 | 15.1 |
| | Cmpd A 134.4 mg | 6 | 2723 | 6 | 2682 | 519.45 | 19.1 |
| Cmax (ng/mL) | Cmpd A 7 mg | 5 | 16.90 | 5 | 16.70 | 2.8896 | 17.1 |
| | Cmpd A 14 mg | 6 | 37.05 | 6 | 36.83 | 4.3976 | 11.9 |
| | Cmpd A 28 mg | 5 | 58.08 | 5 | 57.56 | 8.2309 | 14.2 |
| | Cmpd A 56 mg | 6 | 123.2 | 6 | 122.6 | 13.014 | 10.6 |
| | Cmpd A 112 mg | 6 | 238.3 | 6 | 235.2 | 40.712 | 17.1 |
| | Cmpd A 134.4 mg | 6 | 308.3 | 6 | 304.9 | 50.702 | 16.4 |
| Ceoi (ng/mL) | Cmpd A 7 mg | 5 | 13.50 | 5 | 13.37 | 2.0174 | 14.9 |
| | Cmpd A 14 mg | 6 | 30.08 | 6 | 29.81 | 4.2579 | 14.2 |
| | Cmpd A 28 mg | 5 | 52.52 | 5 | 52.08 | 7.2344 | 13.8 |
| | Cmpd A 56 mg | 6 | 115.2 | 6 | 114.6 | 13.303 | 11.6 |
| | Cmpd A 112 mg | 6 | 215.8 | 6 | 213.8 | 31.714 | 14.7 |
| | Cmpd A 134.4 mg | 6 | 291.8 | 6 | 290.3 | 32.096 | 11.0 |
| AUC24 (h*ng/mL) | Cmpd A 7 mg | 5 | 141.6 | 5 | 140.1 | 22.479 | 15.9 |
| | Cmpd A 14 mg | 6 | 325.8 | 6 | 323.1 | 45.793 | 14.1 |
| | Cmpd A 28 mg | 5 | 518.8 | 5 | 513.3 | 81.392 | 15.7 |
| | Cmpd A 56 mg | 6 | 1067 | 6 | 1055 | 178.49 | 16.7 |
| | Cmpd A 112 mg | 6 | 2088 | 6 | 2069 | 298.89 | 14.3 |
| | Cmpd A 134.4 mg | 6 | 2693 | 6 | 2655 | 495.32 | 18.4 |

| | | Statistics | | | | |
|---|---|---|---|---|---|---|
| Variable | Treatment | Minimum | Q1 | Median | Q3 | Maximum |
| AUClast (h*ng/mL) | Cmpd A 7 mg | 104 | 137.0 | 142.0 | 150.0 | 165 |
| | Cmpd A 14 mg | 269 | 287.0 | 321.5 | 377.0 | 379 |
| | Cmpd A 28 mg | 394 | 486.0 | 543.0 | 572.0 | 599 |
| | Cmpd A 56 mg | 896 | 930.0 | 1042 | 1100 | 1390 |
| | Cmpd A 112 mg | 1580 | 2000 | 2130 | 2200 | 2490 |
| | Cmpd A 134.4 mg | 2000 | 2450 | 2670 | 2930 | 3440 |
| AUCinf (h*ng/mL) | Cmpd A 7 mg | 108 | 139.0 | 146.0 | 155.0 | 169 |
| | Cmpd A 14 mg | 272 | 288.0 | 322.5 | 386.0 | 397 |
| | Cmpd A 28 mg | 401 | 488.0 | 549.0 | 583.0 | 628 |
| | Cmpd A 56 mg | 900 | 938.0 | 1049 | 1110 | 1420 |
| | Cmpd A 112 mg | 1580 | 2020 | 2155 | 2210 | 2560 |
| | Cmpd A 134.4 mg | 2010 | 2480 | 2685 | 2950 | 3530 |
| Cmax (ng/mL) | Cmpd A 7 mg | 13.2 | 15.40 | 16.50 | 18.80 | 20.6 |
| | Cmpd A 14 mg | 31.8 | 33.00 | 37.00 | 40.00 | 43.5 |
| | Cmpd A 28 mg | 44.4 | 57.80 | 59.30 | 63.70 | 65.2 |
| | Cmpd A 56 mg | 107 | 114.0 | 121.5 | 131.0 | 144 |
| | Cmpd A 112 mg | 171 | 215.0 | 249.5 | 255.0 | 290 |
| | Cmpd A 134.4 mg | 241 | 268.0 | 314.5 | 324.0 | 388 |
| Ceoi (ng/mL) | Cmpd A 7 mg | 10.2 | 13.20 | 13.90 | 15.00 | 15.2 |
| | Cmpd A 14 mg | 22.8 | 28.40 | 30.80 | 33.50 | 34.2 |
| | Cmpd A 28 mg | 40.6 | 51.00 | 55.30 | 57.40 | 58.3 |
| | Cmpd A 56 mg | 102 | 104.0 | 112.5 | 122.0 | 138 |
| | Cmpd A 112 mg | 169 | 192.0 | 221.0 | 245.0 | 247 |
| | Cmpd A 134.4 mg | 241 | 268.0 | 300.5 | 311.0 | 330 |
| AUC24 (h*ng/mL) | Cmpd A 7 mg | 107 | 140.0 | 142.0 | 150.0 | 169 |
| | Cmpd A 14 mg | 269 | 287.0 | 321.5 | 377.0 | 379 |
| | Cmpd A 28 mg | 394 | 486.0 | 543.0 | 572.0 | 599 |
| | Cmpd A 56 mg | 896 | 930.0 | 1042 | 1100 | 1390 |
| | Cmpd A 112 mg | 1580 | 2000 | 2130 | 2200 | 2490 |
| | Cmpd A 134.4 mg | 2000 | 2450 | 2670 | 2930 | 3440 |

| | | Statistics | | | | | | | |
|---|---|---|---|---|---|---|---|---|---|
| Variable | Treatment | N | Mean | SD | Minimum | Q1 | Median | Q3 | Maximum |
| tmax (h) | Cmpd A 7 mg | 5 | 3.000 | 0.7071 | 2.00 | 3.000 | 3.000 | 3.000 | 4.00 |
| | Cmpd A 14 mg | 6 | 3.667 | 0.5164 | 3.00 | 3.000 | 4.000 | 4.000 | 4.00 |
| | Cmpd A 28 mg | 5 | 3.000 | 0.7071 | 2.00 | 3.000 | 3.000 | 3.000 | 4.00 |
| | Cmpd A 56 mg | 6 | 4.500 | 2.2583 | 3.00 | 3.000 | 4.000 | 4.000 | 9.00 |
| | Cmpd A 112 mg | 6 | 4.250 | 2.5249 | 1.50 | 3.000 | 4.000 | 4.000 | 9.00 |
| | Cmpd A 134.4 mg | 6 | 6.333 | 2.9439 | 3.00 | 4.000 | 6.500 | 9.000 | 9.00 |

| | | Statistics | | | | | | | | |
|---|---|---|---|---|---|---|---|---|---|---|
| Variable | Treatment | N | Mean | SD | CV | Minimum | Q1 | Median | Q3 | Maximum |
| t½z (h) | Cmpd A 7 mg | 5 | 4.120 | 1.9190 | 46.6 | 2.27 | 2.830 | 3.220 | 5.560 | 6.72 |
| | Cmpd A 14 mg | 6 | 4.498 | 1.7725 | 39.4 | 2.77 | 3.390 | 4.170 | 4.640 | 7.85 |
| | Cmpd A 28 mg | 5 | 5.142 | 1.8060 | 35.1 | 3.04 | 4.000 | 5.060 | 5.860 | 7.75 |
| | Cmpd A 56 mg | 6 | 3.860 | 0.6478 | 16.8 | 3.13 | 3.540 | 3.760 | 3.910 | 5.06 |
| | Cmpd A 112 mg | 6 | 3.788 | 1.0424 | 27.5 | 2.83 | 3.080 | 3.370 | 4.580 | 5.50 |
| | Cmpd A 134.4 mg | 6 | 3.943 | 0.9492 | 24.1 | 2.66 | 3.520 | 3.760 | 4.500 | 5.46 |
| Vss (L) | Cmpd A 7 mg | 5 | 82.88 | 29.543 | 35.6 | 44.5 | 57.90 | 100.0 | 102.0 | 110 |
| | Cmpd A 14 mg | 6 | 77.07 | 22.804 | 29.6 | 52.8 | 63.40 | 72.65 | 81.90 | 119 |
| | Cmpd A 28 mg | 5 | 116.1 | 35.215 | 30.3 | 80.3 | 89.00 | 105.0 | 146.0 | 160 |
| | Cmpd A 56 mg | 6 | 91.40 | 14.122 | 15.5 | 75.5 | 79.70 | 89.10 | 102.0 | 113 |
| | Cmpd A 112 mg | 6 | 97.83 | 22.194 | 22.7 | 58.0 | 89.00 | 104.5 | 109.0 | 122 |
| | Cmpd A 134.4 mg | 6 | 88.47 | 12.476 | 14.1 | 75.0 | 78.20 | 86.95 | 95.70 | 108 |
| Vz (L) | Cmpd A 7 mg | 5 | 292.2 | 124.03 | 42.4 | 165 | 169.0 | 303.0 | 386.0 | 438 |
| | Cmpd A 14 mg | 6 | 271.7 | 79.238 | 29.2 | 168 | 238.0 | 253.0 | 318.0 | 400 |
| | Cmpd A 28 mg | 5 | 391.8 | 117.09 | 29.9 | 251 | 294.0 | 406.0 | 498.0 | 510 |
| | Cmpd A 56 mg | 6 | 290.7 | 31.156 | 10.7 | 253 | 273.0 | 281.5 | 318.0 | 337 |
| | Cmpd A 112 mg | 6 | 288.8 | 55.185 | 19.1 | 207 | 241.0 | 301.0 | 336.0 | 347 |
| | Cmpd A 134.4 mg | 6 | 284.7 | 65.265 | 22.9 | 207 | 232.0 | 275.5 | 353.0 | 365 |
| CL (L/h) | Cmpd A 7 mg | 5 | 50.00 | 9.0785 | 18.2 | 41.4 | 45.10 | 48.10 | 50.30 | 65.1 |
| | Cmpd A 14 mg | 6 | 43.07 | 6.4856 | 15.1 | 35.3 | 36.30 | 43.40 | 48.60 | 51.4 |
| | Cmpd A 28 mg | 5 | 54.20 | 9.9541 | 18.4 | 44.6 | 48.10 | 51.00 | 57.40 | 69.9 |
| | Cmpd A 56 mg | 6 | 53.10 | 8.2202 | 15.5 | 39.3 | 50.50 | 53.45 | 59.70 | 62.2 |
| | Cmpd A 112 mg | 6 | 54.10 | 9.0437 | 16.7 | 43.7 | 50.70 | 52.00 | 55.50 | 70.7 |
| | Cmpd A 134.4 mg | 6 | 50.90 | 9.8018 | 19.3 | 38.1 | 45.60 | 50.35 | 54.30 | 66.7 |
| AUCinf D (h*ng/mL/mg) | Cmpd A 7 mg | 5 | 20.48 | 3.2522 | 15.9 | 15.4 | 19.90 | 20.80 | 22.20 | 24.1 |
| | Cmpd A 14 mg | 6 | 23.70 | 3.6475 | 15.4 | 19.5 | 20.60 | 23.05 | 27.60 | 28.4 |
| | Cmpd A 28 mg | 5 | 18.90 | 3.1528 | 16.7 | 14.3 | 17.40 | 19.60 | 20.80 | 22.4 |
| | Cmpd A 56 mg | 6 | 19.27 | 3.3572 | 17.4 | 16.1 | 16.80 | 18.75 | 19.80 | 25.4 |
| | Cmpd A 112 mg | 6 | 18.87 | 2.8682 | 15.2 | 14.1 | 18.00 | 19.25 | 19.70 | 22.9 |
| | Cmpd A 134.4 mg | 6 | 20.23 | 3.8339 | 18.9 | 15.0 | 18.40 | 19.95 | 21.90 | 26.2 |
| Cmax D (ng/mL/mg) | Cmpd A 7 mg | 5 | 2.416 | 0.41113 | 17.0 | 1.89 | 2.200 | 2.360 | 2.690 | 2.94 |
| | Cmpd A 14 mg | 6 | 2.647 | 0.31532 | 11.9 | 2.27 | 2.360 | 2.640 | 2.860 | 3.11 |
| | Cmpd A 28 mg | 5 | 2.076 | 0.29348 | 14.1 | 1.59 | 2.060 | 2.120 | 2.280 | 2.33 |
| | Cmpd A 56 mg | 6 | 2.200 | 0.23160 | 10.5 | 1.91 | 2.040 | 2.170 | 2.340 | 2.57 |
| | Cmpd A 112 mg | 6 | 2.130 | 0.36304 | 17.0 | 1.53 | 1.920 | 2.230 | 2.280 | 2.59 |
| | Cmpd A 134.4 mg | 6 | 2.293 | 0.37977 | 16.6 | 1.79 | 1.990 | 2.340 | 2.410 | 2.89 |

| | | Statistics | | | | | |
|---|---|---|---|---|---|---|---|
| | | | | N of Geo | | | |
| Variable | Treatment | N | Mean | Mean | Geo Mean | SD | CV |
| AUClast (h*ng/mL) | Cmpd A 180 mg | 6 | 2998 | 6 | 2994 | 164.73 | 5.5 |
| | Cmpd A 240 mg | 6 | 4585 | 6 | 4542 | 698.25 | 15.2 |
| AUCinf (h*ng/mL) | Cmpd A 180 mg | 6 | 3017 | 6 | 3013 | 160.21 | 5.3 |
| | Cmpd A 240 mg | 6 | 4627 | 6 | 4585 | 689.28 | 14.9 |
| Cmax (ng/mL) | Cmpd A 180 mg | 6 | 345.2 | 6 | 344.5 | 23.549 | 6.8 |
| | Cmpd A 240 mg | 6 | 512.2 | 6 | 506.3 | 87.027 | 17.0 |
| Ceoi (ng/mL) | Cmpd A 180 mg | 6 | 325.2 | 6 | 324.0 | 30.182 | 9.3 |
| | Cmpd A 240 mg | 6 | 497.2 | 6 | 493.2 | 70.335 | 14.1 |
| AUC24 (h*ng/mL) | Cmpd A 180 mg | 6 | 2998 | 6 | 2994 | 164.73 | 5.5 |
| | Cmpd A 240 mg | 6 | 4585 | 6 | 4542 | 698.25 | 15.2 |

| | | Statistics | | | | |
|---|---|---|---|---|---|---|
| Variable | Treatment | Minimum | Q1 | Median | Q3 | Maximum |
| AUClast (h*ng/mL) | Cmpd A 180 mg | 2710 | 2950 | 3035 | 3050 | 3210 |
| | Cmpd A 240 mg | 3860 | 4010 | 4395 | 5320 | 5530 |
| AUCinf (h*ng/mL) | Cmpd A 180 mg | 2730 | 2990 | 3050 | 3060 | 3220 |
| | Cmpd A 240 mg | 3930 | 4060 | 4430 | 5330 | 5580 |
| Cmax (ng/mL) | Cmpd A 180 mg | 312 | 318.0 | 358.0 | 361.0 | 364 |
| | Cmpd A 240 mg | 423 | 452.0 | 483.0 | 582.0 | 650 |
| Ceoi (ng/mL) | Cmpd A 180 mg | 285 | 311.0 | 316.5 | 358.0 | 364 |
| | Cmpd A 240 mg | 417 | 452.0 | 481.0 | 539.0 | 613 |
| AUC24 (h*ng/mL) | Cmpd A 180 mg | 2710 | 2950 | 3035 | 3050 | 3210 |
| | Cmpd A 240 mg | 3860 | 4010 | 4395 | 5320 | 5530 |

| | | Statistics | | | | | | | |
|---|---|---|---|---|---|---|---|---|---|
| Variable | Treatment | N | Mean | SD | Minimum | Q1 | Median | Q3 | Maximum |
| tmax (h) | Cmpd A 180 mg | 6 | 6.333 | 2.9439 | 3.00 | 4.000 | 6.500 | 9.000 | 9.00 |
| | Cmpd A 240 mg | 6 | 5.333 | 2.5033 | 3.00 | 4.000 | 4.000 | 8.000 | 9.00 |

Statistics

| Variable | Treatment | N | Mean | SD | CV | Minimum | Q1 | Median | Q3 | Maximum |
|---|---|---|---|---|---|---|---|---|---|---|
| t½z (h) | Cmpd A 180 mg | 6 | 3.363 | 0.8523 | 25.3 | 2.43 | 2.980 | 3.225 | 3.360 | 4.96 |
| | Cmpd A 240 mg | 6 | 4.033 | 1.2843 | 31.8 | 2.25 | 3.090 | 4.230 | 4.370 | 6.03 |
| Vss (L) | Cmpd A 180 mg | 6 | 90.75 | 24.140 | 26.6 | 65.6 | 76.00 | 81.45 | 111.0 | 129 |
| | Cmpd A 240 mg | 6 | 97.48 | 28.378 | 29.1 | 56.1 | 75.30 | 99.25 | 125.0 | 130 |
| Vz (L) | Cmpd A 180 mg | 6 | 290.8 | 77.783 | 26.7 | 207 | 251.0 | 268.5 | 320.0 | 430 |
| | Cmpd A 240 mg | 6 | 313.0 | 128.81 | 41.2 | 146 | 254.0 | 293.0 | 361.0 | 531 |
| CL (L/h) | Cmpd A 180 mg | 6 | 59.83 | 3.3381 | 5.6 | 55.9 | 58.80 | 59.10 | 60.10 | 66.0 |
| | Cmpd A 240 mg | 6 | 52.78 | 7.4882 | 14.2 | 43.0 | 45.00 | 54.35 | 59.00 | 61.0 |
| AUCinf D (h*ng/mL/mg) | Cmpd A 180 mg | 6 | 16.73 | 0.91360 | 5.5 | 15.1 | 16.60 | 16.90 | 17.00 | 17.9 |
| | Cmpd A 240 mg | 6 | 19.28 | 2.8799 | 14.9 | 16.4 | 16.90 | 18.45 | 22.20 | 23.3 |
| Cmax D (ng/mL/mg) | Cmpd A 180 mg | 6 | 1.918 | 0.13152 | 6.9 | 1.73 | 1.770 | 1.990 | 2.010 | 2.02 |
| | Cmpd A 240 mg | 6 | 2.133 | 0.36533 | 17.1 | 1.76 | 1.880 | 2.010 | 2.430 | 2.71 |

TABLE 7
Summary of Plasma Concentrations by Visit (Cohort 5, 6, 7)
<Pharmacokinetic Analysis Set>

| | | Visit | | | | | | | | | |
|---|---|---|---|---|---|---|---|---|---|---|---|
| | | | | | | | | 0.17 hr | 0.5 hr | 2 hr | |
| | | | | | | | | Afte | Afte | After | |
| Analyte/ | | | | | | | | End of | End of | End of | At Bed |
| Treatment | Statistics | Predose | 1 hr | 2 hr | 4 hr | 6 hr | 9 hr | Infusion | Infusion | Infusions | Time |
| Compound A (ng/mL) | | | | | | | | | | | |
| Compound A 5 mg | N | 6 | 6 | 6 | 6 | 6 | 6 | 5 | 5 | 5 | 5 |
| | Mean | 0.000 | 6.383 | 8.747 | 10.09 | 7.905 | 9.643 | 5.888 | 4.424 | 2.358 | 1.071 |
| | SD | 0.0000 | 1.3394 | 1.0704 | 2.3062 | 1.8301 | 1.0265 | 1.8385 | 1.4571 | 0.77115 | 0.43704 |
| | Minimum | 0.00 | 4.33 | 7.07 | 7.59 | 5.71 | 7.73 | 4.11 | 2.76 | 1.37 | 0.740 |
| | Q1 | 0.000 | 5.900 | 7.820 | 8.010 | 6.000 | 9.320 | 4.810 | 3.860 | 2.100 | 0.7900 |
| | Median | 0.000 | 6.255 | 9.150 | 9.615 | 7.970 | 9.955 | 5.520 | 3.990 | 2.260 | 0.8550 |
| | Q3 | 0.000 | 7.340 | 9.480 | 12.50 | 9.580 | 10.40 | 6.110 | 4.840 | 2.570 | 1.180 |
| | Maximum | 0.00 | 8.22 | 9.81 | 13.2 | 10.2 | 10.5 | 8.89 | 6.67 | 3.49 | 1.79 |
| Compound A 11.2 mg | N | 4 | 4 | 4 | 4 | 4 | 4 | 4 | 4 | 4 | 4 |
| | Mean | 0.000 | 14.38 | 17.30 | 21.50 | 19.53 | 21.23 | 12.38 | 9.545 | 4.838 | 1.923 |
| | SD | 0.0000 | 1.2712 | 1.7795 | 2.6331 | 1.3326 | 1.7896 | 1.8927 | 2.1740 | 0.62142 | 0.47282 |
| | Minimum | 0.00 | 12.9 | 15.4 | 18.3 | 17.7 | 18.6 | 10.8 | 7.26 | 3.94 | 1.43 |
| | Q1 | 0.000 | 13.55 | 16.20 | 19.70 | 18.70 | 20.15 | 11.15 | 8.225 | 4.420 | 1.540 |
| | Median | 0.000 | 14.30 | 17.05 | 21.50 | 19.75 | 21.85 | 11.80 | 9.210 | 5.070 | 1.890 |
| | Q3 | 0.000 | 15.20 | 18.40 | 23.30 | 20.35 | 22.30 | 13.60 | 10.87 | 5.255 | 2.305 |
| | Maximum | 0.00 | 16.0 | 19.7 | 24.7 | 20.9 | 22.6 | 15.1 | 12.5 | 5.27 | 2.48 |
| Compound A 44.8 mg | N | 4 | 4 | 4 | 4 | 4 | 4 | 4 | 4 | 4 | 4 |
| | Mean | 0.000 | 61.53 | 74.08 | 96.00 | 90.00 | 96.25 | 68.70 | 53.93 | 25.85 | 11.37 |
| | SD | 0.0000 | 18.920 | 16.445 | 22.699 | 20.132 | 23.643 | 17.568 | 16.257 | 11.637 | 6.8086 |
| | Minimum | 0.00 | 44.7 | 52.4 | 73.2 | 68.4 | 68.5 | 53.3 | 35.7 | 11.2 | 4.26 |
| | Q1 | 0.000 | 49.10 | 62.20 | 78.50 | 74.50 | 80.30 | 56.30 | 42.85 | 17.10 | 6.685 |
| | Median | 0.000 | 56.50 | 76.35 | 92.90 | 88.30 | 95.25 | 64.15 | 52.50 | 26.85 | 10.36 |
| | Q3 | 0.000 | 73.95 | 85.95 | 113.5 | 105.5 | 112.2 | 81.10 | 65.00 | 34.60 | 16.05 |
| | Maximum | 0.00 | 88.4 | 91.2 | 125 [symbol] | 115 [symbol] | 126 [symbol] | 93.2 | 75.0 | 38.5 | 20.5 |

[symbol]

indicates data missing or illegible when filed

TABLE 8
Summary of Pharmacokinetic Parameters of Cmpd A (Cohort 5, 6, 7)
<Pharmacokinetic Analysis Set>

| | | Statistics | | | | | | | | | | |
|---|---|---|---|---|---|---|---|---|---|---|---|---|
| | | | | N of Geo | | | | | | | | |
| Variable | Treatment | N | Mean | Mean | Geo Mean | SD | CV | Minimum | Q1 | Median | Q3 | Maximum |
| AUClast (h*ng/mL) | Cmpd A 5 mg | 4 | 87.15 | 4 | 86.15 | 14.777 | 17.0 | 67.3 | 77.75 | 89.15 | 96.55 | 103 |
| | Cmpd A 11.2 mg | 4 | 199.0 | 4 | 198.7 | 13.540 | 6.8 | 182 | 190.0 | 199.5 | 208.0 | 215 |
| | Cmpd A 44.8 mg | 4 | 941.0 | 4 | 910.8 | 275.10 | 29.2 | 636 | 740.0 | 919.0 | 1142 | 1290 |
| AUCinf (h*ng/mL) | Cmpd A 5 mg | 4 | 89.28 | 4 | 88.45 | 13.682 | 15.3 | 70.9 | 80.75 | 91.10 | 97.80 | 104 |
| | Cmpd A 11.2 mg | 4 | 201.0 | 4 | 200.7 | 13.216 | 6.6 | 184 | 192.0 | 202.0 | 210.0 | 216 |
| | Cmpd A 44.8 mg | 4 | 959.3 | 4 | 925.3 | 296.03 | 30.9 | 638 | 743.5 | 929.5 | 1175 | 1340 |
| Cmax (ng/mL) | Cmpd A 5 mg | 5 | 10.25 | 5 | 10.10 | 1.9527 | 19.1 | 7.73 | 9.920 | 9.990 | 10.40 | 13.2 |
| | Cmpd A 11.2 mg | 4 | 21.98 | 4 | 21.86 | 2.5303 | 11.5 | 18.6 | 20.30 | 22.30 | 23.65 | 24.7 |
| | Cmpd A 44.8 mg | 4 | 98.33 | 4 | 96.48 | 21.981 | 22.4 | 73.2 | 82.65 | 97.05 | 114.0 | 126 |
| Ceoi (ng/mL) | Cmpd A 5 mg | 5 | 9.708 | 5 | 9.649 | 1.1339 | 11.7 | 7.73 | 9.920 | 9.990 | 10.40 | 10.5 |
| | Cmpd A 11.2 mg | 4 | 21.23 | 4 | 21.17 | 1.7896 | 8.4 | 18.6 | 20.15 | 21.85 | 22.30 | 22.6 |
| | Cmpd A 44.8 mg | 4 | 96.25 | 4 | 94.04 | 23.643 | 24.6 | 68.5 | 80.30 | 95.25 | 112.2 | 126 |
| AUC24 (h*ng/mL) | Cmpd A 5 mg | 4 | 88.80 | 4 | 88.00 | 13.388 | 15.1 | 70.7 | 80.40 | 90.75 | 97.20 | 103 |
| | Cmpd A 11.2 mg | 4 | 199.0 | 4 | 198.7 | 13.540 | 6.8 | 182 | 190.0 | 199.5 | 208.0 | 215 |
| | Cmpd A 44.8 mg | 4 | 941.0 | 4 | 910.8 | 275.10 | 29.2 | 636 | 740.0 | 919.0 | 1142 | 1290 |

| | | Statistics | | | | | | | |
|---|---|---|---|---|---|---|---|---|---|
| Variable | Treatment | N | Mean | SD | Minimum | Q1 | Median | Q3 | Maximum |
| tmax (h) | Cmpd A 5 mg | 5 | 8.000 | 2.2361 | 4.00 | 9.000 | 9.000 | 9.000 | 9.00 |
| | Cmpd A 11.2 mg | 4 | 7.750 | 2.5000 | 4.00 | 6.500 | 9.000 | 9.000 | 9.00 |
| | Cmpd A 44.8 mg | 4 | 6.500 | 2.8868 | 4.00 | 4.000 | 6.500 | 9.000 | 9.00 |

| | | Statistics | | | | | | | | |
|---|---|---|---|---|---|---|---|---|---|---|
| Variable | Treatment | N | Mean | SD | CV | Minimum | Q1 | Median | Q3 | Maximum |
| t½z (h) | Cmpd A 5 mg | 4 | 3.465 | 1.0998 | 31.7 | 2.23 | 2.685 | 3.380 | 4.245 | 4.87 |
| | Cmpd A 11.2 mg | 4 | 3.923 | 1.0265 | 26.2 | 2.74 | 3.070 | 4.020 | 4.775 | 4.91 |
| | Cmpd A 44.8 mg | 4 | 4.130 | 1.1981 | 29.0 | 3.13 | 3.160 | 3.885 | 5.100 | 5.62 |
| Vss (L) | Cmpd A 5 mg | 4 | 111.7 | 26.464 | 23.7 | 88.0 | 88.95 | 110.5 | 134.5 | 138 |
| | Cmpd A 11.2 mg | 4 | 114.1 | 14.964 | 13.1 | 96.4 | 104.7 | 113.5 | 123.5 | 133 |
| | Cmpd A 44.8 mg | 4 | 116.8 | 7.8049 | 6.7 | 110 | 110.0 | 116.5 | 123.5 | 124 |
| Vz (L) | Cmpd A 5 mg | 4 | 282.3 | 88.729 | 31.4 | 177 | 213.5 | 284.5 | 351.0 | 383 |
| | Cmpd A 11.2 mg | 4 | 316.8 | 84.263 | 26.6 | 205 | 252.0 | 336.5 | 381.5 | 389 |
| | Cmpd A 44.8 mg | 4 | 281.3 | 35.855 | 12.7 | 238 | 254.5 | 282.0 | 308.0 | 323 |
| CL (L/h) | Cmpd A 5 mg | 4 | 57.05 | 9.5577 | 16.8 | 47.9 | 51.25 | 54.90 | 62.85 | 70.5 |
| | Cmpd A 11.2 mg | 4 | 55.90 | 3.7692 | 6.7 | 51.8 | 53.40 | 55.45 | 58.40 | 60.9 |
| | Cmpd A 44.8 mg | 4 | 50.18 | 15.533 | 31.0 | 33.4 | 38.85 | 48.55 | 61.50 | 70.2 |
| AUCinf D (h*ng/mL/mg) | Cmpd A 5 mg | 4 | 17.88 | 2.7621 | 15.5 | 14.2 | 16.15 | 18.20 | 19.60 | 20.9 |
| | Cmpd A 11.2 mg | 4 | 17.95 | 1.1958 | 6.7 | 16.4 | 17.15 | 18.05 | 18.75 | 19.3 |
| | Cmpd A 44.8 mg | 4 | 21.45 | 6.6581 | 31.0 | 14.2 | 16.60 | 20.80 | 26.30 | 30.0 |
| Cmax D (ng/mL/mg) | Cmpd A 5 mg | 5 | 2.050 | 0.38936 | 19.0 | 1.55 | 1.980 | 2.000 | 2.080 | 2.64 |
| | Cmpd A 11.2 mg | 4 | 1.963 | 0.22809 | 11.6 | 1.66 | 1.810 | 1.990 | 2.115 | 2.21 |
| | Cmpd A 44.8 mg | 4 | 2.195 | 0.49088 | 22.4 | 1.63 | 1.845 | 2.170 | 2.545 | 2.81 |

TABLE 9
Summary of Plasma Concentrations by Visit (Cohort 4)
<Pharmacokinetic Analysis Set>

| | | Visit | | | | | | | | | | |
|---|---|---|---|---|---|---|---|---|---|---|---|---|
| | | | | | | | | | | | | 0.17 hr |
| | | | | | | | | | | | | After |
| | | | | | | | | | | | | End of |
| Analyte | Statistics | Predose | 0.5 hr | 1 hr | 1.5 hr | 2 hr | 3 hr | 4 hr | 6 hr | 8 hr | 9 hr | Infusion |
| Cmpd A (ng/mL) | | | | | | | | | | | | |
| | N | 4 | 4 | 4 | 4 | 4 | 4 | 4 | 4 | 4 | 4 | 4 |
| | Mean | 0.000 | 119.2 | 161.5 | 193.3 | 186.5 | 215.8 | 234.0 | 233.8 | 176.8 | 184.0 | 113.0 |
| | SD | 0.0000 | 30.217 | 27.441 | 34.568 | 24.076 | 9.8784 | 28.671 | 22.441 | 26.005 | 15.875 | 17.603 |
| | Minimum | 0.00 | 87.8 | 137 | 155 | 161 | 202 | 213 | 216 | 157 | 169 | 94.9 |
| | Q1 | 0.000 | 96.40 | 140.0 | 164.5 | 171.0 | 209.0 | 216.0 | 217.5 | 161.5 | 171.0 | 101.5 |
| | Median | 0.000 | 115.5 | 156.0 | 194.5 | 183.0 | 218.0 | 223.5 | 227.0 | 167.5 | 182.0 | 110.0 |
| | Q3 | 0.000 | 142.0 | 183.0 | 222.0 | 202.0 | 222.5 | 252.0 | 250.0 | 192.0 | 197.0 | 124.5 |
| | Maximum | 0.00 | 158 | 197 | 229 | 219 | 225 | 276 | 265 | 215 | 203 | 137 |

| | | Visit | | | | | |
|---|---|---|---|---|---|---|---|
| | | 0.33 hr | 0.5 hr | 0.75 hr | 1 hr | 1.5 hr | 2 hr |
| | | After | After | After | After | After | After |
| | | End of | End of | End of | End of | End of | End of |
| Analyte | Statistics | Infusion | Infusion | Infusion | Infusion | Infusions | Infusions |
| Cmpd A (ng/mL) | | | | | | | |
| | N | 4 | 4 | 4 | 4 | 4 | 4 |
| | Mean | 92.68 | 85.58 | 72.88 | 60.93 | 46.63 | 30.58 |
| | SD | 7.2431 | 8.2148 | 5.9539 | 2.6260 | 3.3718 | 3.4587 |
| | Minimum | 82.7 | 73.4 | 65.0 | 57.9 | 43.5 | 26.0 |
| | Q1 | 87.35 | 80.75 | 68.80 | 58.75 | 44.50 | 27.95 |
| | Median | 94.70 | 88.85 | 73.60 | 61.15 | 45.80 | 31.25 |
| | Q3 | 98.00 | 90.40 | 76.95 | 63.10 | 48.75 | 33.20 |
| | Maximum | 98.6 | 91.2 | 79.3 | 63.5 | 51.4 | 33.8 |

| | | Visit | | | | |
|---|---|---|---|---|---|---|
| | | 3 hr | 4 hr | 6 hr | 10 hr | 15 hr |
| | | After | After | After | After | After |
| | | End of | End of | End of | End of | End of |
| Analyte | Statistics | Infusions | Infusions | Infusions | Infusions | Infusions |
| Cmpd A (ng/mL) | | | | | | |
| | N | 4 | 4 | 4 | 4 | 4 |
| | Mean | 22.13 | 16.33 | 7.968 | 5.365 | 2.445 |
| | SD | 1.8572 | 2.4102 | 2.3247 | 3.4514 | 2.3070 |
| | Minimum | 19.8 | 13.4 | 5.11 | 2.58 | 1.12 |
| | Q1 | 20.75 | 14.60 | 6.490 | 3.300 | 1.225 |
| | Median | 22.25 | 16.35 | 7.980 | 4.240 | 1.380 |
| | Q3 | 23.50 | 18.05 | 9.445 | 7.430 | 3.665 |
| | Maximum | 24.2 | 19.2 | 10.8 | 10.4 | 5.90 |

TABLE 10
Summary of CSF Concentrations by Visit (Cohort 4)
<Pharmacokinetic Analysis Set>

| | | Visit |
|---|---|---|
| Analyte | Statistics | 6 hr |
| Cerebrospinal Fluid Cmpd A (ng/mL | N | 4 |
| | Mean | 6.730 |
| | SD | 1.9154 |
| | Minimum | 4.66 |
| | Q1 | 5.205 |
| | Median | 6.630 |
| | Q3 | 8.255 |
| | Maximum | 9.00 |

TABLE 11
Summary of Average Sleep Latency in MWT (Cohort 5, 6, 7)
<Pharmacodynamic Analysis Set>
Average Sleep Latency in MWT

| | | Cmpd A | Cmpd A | Cmpd A |
|---|---|---|---|---|
| | Placebo | 5 mg | 11.2 mg | 44.8 mg |
| Statistics | (N = 13) | (N = 6) | (N = 4) | (N = 4) |
| N | 13 | 6 | 4 | 4 |
| Mean | 2.88 | 22.38 | 37.59 | 40.00 |
| SD | 1.646 | 10.125 | 4.813 | 0.000 |
| Minimum | 0.6 | 5.1 | 30.4 | 40.0 |
| Q1 | 1.50 | 20.50 | 35.19 | 40.00 |
| Median | 2.50 | 21.69 | 40.00 | 40.00 |
| Q3 | 4.38 | 32.25 | 40.00 | 40.00 |
| Maximum | 5.8 | 33.0 | 40.0 | 40.0 |

TABLE 13
Summary of Daytime Sleepiness as Assessed by KSS by Visit (Cohort 5, 6, 7)
<Pharmacodynamic Analysis Set>
KSS

| | Placebo | | Cmpd A 5 mg | | Cmpd A 11.2 mg | | Cmpd A 44.8 mg | |
|---|---|---|---|---|---|---|---|---|
| | (N = 13) | | (N = 6) | | (N = 4) | | (N = 4) | |
| | Observed | Change | Observed | Change | Observed | Change | Observed | Change |
| Visit/ | Value | from | Value | from | Value | from | Value | from |
| Statistics | at Visit | Baseline | at Visit | Baseline | at Visit | Baseline | at Visit | Baseline |
| Predose | | | | | | | | |
| N | 13 | | 6 | | 4 | | 4 | |
| Mean | 3.8 | | 5.0 | | 3.8 | | 2.8 | |
| SD | 2.09 | | 2.28 | | 2.22 | | 1.26 | |
| Minimum | 1 | | 1 | | 2 | | 1 | |
| Q1 | 3.0 | | 4.0 | | 2.5 | | 2.0 | |
| Median | 3.0 | | 5.5 | | 3.0 | | 3.0 | |
| Q3 | 6.0 | | 7.0 | | 5.0 | | 3.5 | |
| Maximum | 7 | | 7 | | 7 | | 4 | |
| 1 hr | | | | | | | | |
| N | 13 | 13 | 6 | 6 | 4 | 4 | 4 | 4 |
| Mean | 5.6 | 1.8 | 2.7 | −2.3 | 1.8 | −2.0 | 1.3 | −1.5 |
| SD | 2.36 | 2.03 | 1.86 | 2.58 | 0.96 | 2.83 | 0.50 | 1.29 |
| Minimum | 2 | −1 | 1 | −6 | 1 | −6 | 1 | −3 |
| Q1 | 3.0 | 0.0 | 1.0 | −4.0 | 1.0 | −4.0 | 1.0 | −2.5 |
| Median | 6.0 | 2.0 | 2.5 | −2.5 | 1.5 | −1.0 | 1.0 | −1.5 |
| Q3 | 7.0 | 3.0 | 3.0 | 0.0 | 2.5 | 0.0 | 1.5 | −0.5 |
| Maximum | 9 | 6 | 6 | 1 | 3 | 0 | 2 | 0 |
| 2 hr | | | | | | | | |
| N | 13 | 13 | 6 | 6 | 4 | 4 | 4 | 4 |
| Mean | 5.8 | 2.0 | 2.2 | −2.8 | 2.3 | −1.5 | 1.3 | −1.5 |
| SD | 2.24 | 2.35 | 1.17 | 2.32 | 1.50 | 3.42 | 0.50 | 1.00 |
| Minimum | 1 | −2 | 1 | −6 | 1 | −6 | 1 | −2 |
| Q1 | 5.0 | 0.0 | 1.0 | −5.0 | 1.0 | −4.0 | 1.0 | −2.0 |
| Median | 7.0 | 3.0 | 2.0 | −2.5 | 2.0 | −1.0 | 1.0 | −2.0 |
| Q3 | 7.0 | 4.0 | 3.0 | −1.0 | 3.5 | 1.0 | 1.5 | −1.0 |
| Maximum | 9 | 6 | 4 | 0 | 4 | 2 | 2 | 0 |
| 3 hr | | | | | | | | |
| N | 13 | 13 | 6 | 6 | 4 | 4 | 4 | 4 |
| Mean | 3.8 | 0.0 | 1.8 | −3.2 | 2.5 | −1.3 | 1.8 | −1.0 |
| SD | 2.17 | 1.96 | 0.98 | 2.04 | 1.00 | 2.22 | 0.96 | 0.82 |
| Minimum | 1 | −4 | 1 | −6 | 1 | −4 | 1 | −2 |
| Q1 | 2.0 | −1.0 | 1.0 | −4.0 | 2.0 | −3.0 | 1.0 | −1.5 |
| Median | 3.0 | 0.0 | 1.5 | −3.5 | 3.0 | −1.0 | 1.5 | −1.0 |
| Q3 | 5.0 | 1.0 | 3.0 | −2.0 | 3.0 | 0.5 | 2.5 | −0.5 |
| Maximum | 9 | 3 | 3 | 0 | 3 | 1 | 3 | 0 |
| 4 hr | | | | | | | | |
| N | 13 | 13 | 6 | 6 | 4 | 4 | 4 | 4 |
| Mean | 5.7 | 1.9 | 2.2 | −2.8 | 2.0 | −1.8 | 1.8 | −1.0 |
| SD | 2.02 | 3.17 | 1.60 | 2.04 | 0.82 | 2.36 | 0.96 | 0.82 |
| Minimum | 3 | −3 | 1 | −6 | 1 | −5 | 1 | −2 |
| Q1 | 4.0 | 0.0 | 1.0 | −4.0 | 1.5 | −3.5 | 1.0 | −1.5 |
| Median | 6.0 | 3.0 | 1.5 | −2.5 | 2.0 | −1.0 | 1.5 | −1.0 |
| Q3 | 7.0 | 4.0 | 3.0 | −2.0 | 2.5 | 0.0 | 2.5 | −0.5 |
| Maximum | 9 | 6 | 5 | 0 | 3 | 0 | 3 | 0 |
| 5 hr | | | | | | | | |
| N | 13 | 13 | 6 | 6 | 4 | 4 | 4 | 4 |
| Mean | 4.8 | 1.0 | 2.0 | −3.0 | 2.0 | −1.8 | 1.3 | −1.5 |
| SD | 1.88 | 2.45 | 1.10 | 2.00 | 1.15 | 3.10 | 0.50 | 1.29 |
| Minimum | 2 | −3 | 1 | −6 | 1 | −6 | 1 | −3 |
| Q1 | 3.0 | −1.0 | 1.0 | −4.0 | 1.0 | −4.0 | 1.0 | −2.5 |
| Median | 5.0 | 1.0 | 2.0 | −3.0 | 2.0 | −1.0 | 1.0 | −1.5 |
| Q3 | 6.0 | 3.0 | 3.0 | −2.0 | 3.0 | 0.5 | 1.5 | −0.5 |
| Maximum | 7 | 6 | 3 | 0 | 3 | 1 | 2 | 0 |
| 6 hr | | | | | | | | |
| N | 13 | 13 | 6 | 6 | 4 | 4 | 4 | 4 |
| Mean | 5.0 | 1.2 | 1.8 | −3.2 | 2.3 | −1.5 | 1.5 | −1.3 |
| SD | 2.27 | 3.52 | 0.98 | 2.04 | 1.50 | 3.32 | 0.58 | 1.26 |
| Minimum | 2 | −3 | 1 | −6 | 1 | −6 | 1 | −3 |
| Q1 | 3.0 | −1.0 | 1.0 | −4.0 | 1.0 | −4.0 | 1.0 | −2.0 |
| Median | 4.0 | 1.0 | 1.5 | −3.5 | 2.0 | −0.5 | 1.5 | −1.0 |
| Q3 | 7.0 | 4.0 | 3.0 | −2.0 | 3.5 | 1.0 | 2.0 | −0.5 |
| Maximum | 9 | 8 | 3 | 0 | 4 | 1 | 2 | 0 |
| 7 hr | | | | | | | | |
| N | 13 | 13 | 6 | 6 | 4 | 4 | 4 | 4 |
| Mean | 6.2 | 2.5 | 2.5 | −2.5 | 2.8 | −1.0 | 1.5 | −1.3 |
| SD | 2.20 | 3.50 | 1.38 | 2.07 | 0.50 | 2.16 | 0.58 | 1.26 |
| Minimum | 3 | −3 | 1 | −6 | 2 | −4 | 1 | −3 |
| Q1 | 4.0 | 0.0 | 1.0 | −3.0 | 2.5 | −2.5 | 1.0 | −2.0 |
| Median | 7.0 | 3.0 | 2.5 | −2.5 | 3.0 | −0.5 | 1.5 | −1.0 |
| Q3 | 8.0 | 5.0 | 4.0 | −1.0 | 3.0 | 0.5 | 2.0 | −0.5 |
| Maximum | 9 | 8 | 4 | 0 | 3 | 1 | 2 | 0 |
| 8 hr | | | | | | | | |
| N | 13 | 13 | 6 | 6 | 4 | 4 | 4 | 4 |
| Mean | 5.3 | 1.5 | 2.3 | −2.7 | 2.3 | −1.5 | 2.0 | −0.8 |
| SD | 1.70 | 2.40 | 1.03 | 1.51 | 0.96 | 2.65 | 0.82 | 0.50 |
| Minimum | 3 | −2 | 1 | −4 | 1 | −5 | 1 | −1 |
| Q1 | 4.0 | 0.0 | 1.0 | −4.0 | 1.5 | −3.5 | 1.5 | −1.0 |
| Median | 5.0 | 2.0 | 3.0 | −3.0 | 2.5 | −1.0 | 2.0 | −1.0 |
| Q3 | 6.0 | 2.0 | 3.0 | −2.0 | 3.0 | 0.5 | 2.5 | −0.5 |
| Maximum | 9 | 6 | 3 | 0 | 3 | 1 | 3 | 0 |
| 9 hr | | | | | | | | |
| N | 13 | 13 | 6 | 6 | 4 | 4 | 4 | 4 |
| Mean | 4.1 | 0.3 | 2.5 | −2.5 | 2.5 | −1.3 | 1.5 | −1.3 |
| SD | 2.33 | 2.87 | 1.52 | 1.64 | 1.29 | 2.87 | 1.00 | 1.50 |
| Minimum | 1 | −4 | 1 | −5 | 1 | −5 | 1 | −3 |
| Q1 | 3.0 | −2.0 | 1.0 | −3.0 | 1.5 | −3.5 | 1.0 | −2.5 |
| Median | 3.0 | 0.0 | 2.5 | −2.5 | 2.5 | −0.5 | 1.0 | −1.0 |
| Q3 | 6.0 | 2.0 | 3.0 | −2.0 | 3.5 | 1.0 | 2.0 | 0.0 |
| Maximum | 9 | 6 | 5 | 0 | 4 | 1 | 3 | 0 |
| 10 hr | | | | | | | | |
| N | 13 | 13 | 6 | 6 | 4 | 4 | 4 | 4 |
| Mean | 4.5 | 0.8 | 2.7 | −2.3 | 5.0 | 1.3 | 1.3 | −1.5 |
| SD | 2.40 | 2.59 | 2.34 | 2.25 | 1.41 | 3.59 | 0.50 | 1.29 |
| Minimum | 1 | −4 | 1 | −6 | 3 | −4 | 1 | −3 |
| Q1 | 3.0 | −1.0 | 1.0 | −3.0 | 4.0 | −1.0 | 1.0 | −2.5 |
| Median | 6.0 | 1.0 | 2.0 | −2.5 | 5.5 | 2.5 | 1.0 | −1.5 |
| Q3 | 7.0 | 3.0 | 3.0 | 0.0 | 6.0 | 3.5 | 1.5 | −0.5 |
| Maximum | 7 | 4 | 7 | 0 | 6 | 4 | 2 | 0 |
| 11 hr | | | | | | | | |
| N | 13 | 13 | 6 | 6 | 4 | 4 | 4 | 4 |
| Mean | 3.3 | −0.5 | 3.3 | −1.7 | 4.3 | 0.5 | 1.5 | −1.3 |
| SD | 2.06 | 2.60 | 1.86 | 2.16 | 0.50 | 2.52 | 0.58 | 1.26 |
| Minimum | 1 | −4 | 1 | −5 | 4 | −3 | 1 | −3 |
| Q1 | 2.0 | −3.0 | 2.0 | −3.0 | 4.0 | −1.0 | 1.0 | −2.0 |
| Median | 3.0 | −1.0 | 3.0 | −1.5 | 4.0 | 1.0 | 1.5 | −1.0 |
| Q3 | 5.0 | 1.0 | 5.0 | 0.0 | 4.5 | 2.0 | 2.0 | −0.5 |
| Maximum | 7 | 4 | 6 | 1 | 5 | 3 | 2 | 0 |

TABLE 14
ANOVA of Daytime Sleepiness as Assessed by KSS by Visit (Cohort 5, 6, 7)
<Pharmacodynamic Analysis Set>
KSS

| | | | | | | | | 90% CI | | 95% CI | |
|---|---|---|---|---|---|---|---|---|---|---|---|
| Visit | | Pairs | Estimate | SE | DF | t-value | p-value | Lower | Upper | Lower | Upper |
| 1 | hr | Cmpd A 5 mg - Placebo | −2.000 | 0.6761 | 9.00 | −2.9581 | 0.0160 | −3.239 | −0.761 | −3.529 | −0.471 |
| | | Cmpd A 11.2 mg - Placebo | −3.500 | 0.8281 | 9.00 | −4.2268 | 0.0022 | −5.018 | −1.982 | −5.373 | −1.627 |
| | | Cmpd A 44.8 mg - Placebo | −6.947 | 0.9687 | 9.00 | −7.1722 | <0.0001 | −8.723 | −5.172 | −9.139 | −4.756 |
| 2 | hr | Cmpd A 5 mg - Placebo | −3.500 | 1.1064 | 9.00 | −3.1633 | 0.0115 | −5.528 | −1.472 | −6.003 | −0.997 |
| | | Cmpd A 11.2 mg - Placebo | −3.500 | 1.3551 | 9.00 | −2.5829 | 0.0296 | −5.984 | −1.016 | −6.565 | −0.435 |
| | | Cmpd A 44.8 mg - Placebo | −4.763 | 1.5852 | 9.00 | −3.0048 | 0.0148 | −7.669 | −1.857 | −8.349 | −1.177 |
| 3 | hr | Cmpd A 5 mg - Placebo | −1.333 | 0.6997 | 9.00 | −1.9055 | 0.0891 | −2.616 | −0.051 | −2.916 | 0.250 |
| | | Cmpd A 11.2 mg - Placebo | −1.500 | 0.8570 | 9.00 | −1.7503 | 0.1140 | −3.071 | 0.071 | −3.439 | 0.439 |
| | | Cmpd A 44.8 mg - Placebo | −3.263 | 1.0025 | 9.00 | −3.2551 | 0.0099 | −5.101 | −1.425 | −5.531 | −0.995 |
| 4 | hr | Cmpd A 5 mg - Placebo | −3.333 | 1.2024 | 9.00 | −2.7723 | 0.0217 | −5.537 | −1.129 | −6.053 | −0.613 |
| | | Cmpd A 11.2 mg - Placebo | −4.000 | 1.4726 | 9.00 | −2.7162 | 0.0238 | −6.699 | −1.301 | −7.331 | −0.669 |
| | | Cmpd A 44.8 mg - Placebo | −3.895 | 1.7227 | 9.00 | −2.2609 | 0.0501 | −7.053 | −0.737 | −7.792 | 0.002 |
| 5 | hr | Cmpd A 5 mg - Placebo | −2.667 | 0.9373 | 9.00 | −2.8451 | 0.0192 | −4.385 | −0.949 | −4.787 | −0.546 |
| | | Cmpd A 11.2 mg - Placebo | −3.000 | 1.1479 | 9.00 | −2.6134 | 0.0281 | −5.104 | −0.896 | −5.597 | −0.403 |
| | | Cmpd A 44.8 mg - Placebo | −3.737 | 1.3428 | 9.00 | −2.7828 | 0.0213 | −6.198 | −1.275 | −6.775 | −0.699 |
| 6 | hr | Cmpd A 5 mg - Placebo | −3.500 | 1.2287 | 9.00 | −2.8485 | 0.0191 | −5.752 | −1.248 | −6.280 | −0.720 |
| | | Cmpd A 11.2 mg - Placebo | −3.000 | 1.5049 | 9.00 | −1.9935 | 0.0774 | −5.759 | −0.241 | −6.404 | 0.404 |
| | | Cmpd A 44.8 mg - Placebo | −2.868 | 1.7604 | 9.00 | −1.6294 | 0.1377 | −6.095 | 0.359 | −6.851 | 1.114 |
| 7 | hr | Cmpd A 5 mg - Placebo | −3.500 | 1.3311 | 9.00 | −2.6293 | 0.0274 | −5.940 | −1.060 | −6.511 | −0.489 |
| | | Cmpd A 11.2 mg - Placebo | −3.500 | 1.6303 | 9.00 | −2.1468 | 0.0603 | −6.489 | −0.511 | −7.188 | 0.188 |
| | | Cmpd A 44.8 mg - Placebo | −5.447 | 1.9071 | 9.00 | −2.8563 | 0.0189 | −8.943 | −1.951 | −9.762 | −1.133 |
| 8 | hr | Cmpd A 5 mg - Placebo | −2.667 | 0.9114 | 9.00 | −2.9258 | 0.0169 | −4.337 | −0.996 | −4.729 | −0.605 |
| | | Cmpd A 11.2 mg - Placebo | −2.500 | 1.1163 | 9.00 | −2.2396 | 0.0519 | −4.546 | −0.454 | −5.025 | 0.025 |
| | | Cmpd A 44.8 mg - Placebo | −4.632 | 1.3058 | 9.00 | −3.5468 | 0.0062 | −7.025 | −2.238 | −7.586 | −1.678 |
| 9 | hr | Cmpd A 5 mg - Placebo | −0.667 | 0.8727 | 9.00 | −0.7639 | 0.4645 | −2.266 | 0.933 | −2.641 | 1.307 |
| | | Cmpd A 11.2 mg - Placebo | −2.000 | 1.0688 | 9.00 | −1.8713 | 0.0941 | −3.959 | −0.041 | −4.418 | 0.418 |
| | | Cmpd A 44.8 mg - Placebo | −4.579 | 1.2503 | 9.00 | −3.6624 | 0.0052 | −6.871 | −2.287 | −7.407 | −1.751 |
| 10 | hr | Cmpd A 5 mg - Placebo | −1.167 | 0.7992 | 9.00 | −1.4598 | 0.1784 | −2.632 | 0.298 | −2.975 | 0.641 |
| | | Cmpd A 11.2 mg - Placebo | −1.500 | 0.9788 | 9.00 | −1.5325 | 0.1598 | −3.294 | 0.294 | −3.714 | 0.714 |
| | | Cmpd A 44.8 mg - Placebo | −2.342 | 1.1450 | 9.00 | −2.0455 | 0.0711 | −4.441 | −0.243 | −4.932 | 0.248 |
| 11 | hr | Cmpd A 5 mg - Placebo | 0.667 | 0.6301 | 9.00 | 1.0581 | 0.3176 | −0.488 | 1.822 | −0.759 | 2.092 |
| | | Cmpd A 11.2 mg - Placebo | −1.000 | 0.7717 | 9.00 | −1.2958 | 0.2273 | −2.415 | 0.415 | −2.746 | 0.746 |
| | | Cmpd A 44.8 mg - Placebo | −0.737 | 0.9027 | 9.00 | −0.8162 | 0.4354 | −2.392 | 0.918 | −2.779 | 1.305 |

Note:
The analysis was performed using the GLM procedure because asymptotic variance matrices of covariance parameter estimates produced by the MIXED procedure were singular in some of the ANOVA models.

TABLE 15
ANOVA of Change from Baseline in Daytime Sleepiness as Assessed by KSS by Visit (Cohort 5, 6, 7)
<Pharmacodynamic Analysis Set>
KSS

| | | | | | | | | 90% CI | | 95% CI | |
|---|---|---|---|---|---|---|---|---|---|---|---|
| Visit | | Pairs | Estimate | SE | DF | t-value | p-value | Lower | Upper | Lower | Upper |
| 1 | hr | Cmpd A 5 mg - Placebo | −2.833 | 1.1008 | 9.00 | −2.5738 | 0.0300 | −4.851 | −0.815 | −5.324 | −0.343 |
| | | Cmpd A 11.2 mg - Placebo | −4.500 | 1.3482 | 9.00 | −3.3377 | 0.0087 | −6.971 | −2.029 | −7.550 | −1.450 |
| | | Cmpd A 44.8 mg - Placebo | −5.237 | 1.5772 | 9.00 | −3.3204 | 0.0089 | −8.128 | −2.346 | −8.805 | −1.669 |
| 2 | hr | Cmpd A 5 mg - Placebo | −4.333 | 1.4274 | 9.00 | −3.0359 | 0.0141 | −6.950 | −1.717 | −7.562 | −1.104 |
| | | Cmpd A 11.2 mg - Placebo | −4.500 | 1.7482 | 9.00 | −2.5741 | 0.0300 | −7.705 | −1.295 | −8.455 | −0.545 |
| | | Cmpd A 44.8 mg - Placebo | −3.053 | 2.0450 | 9.00 | −1.4927 | 0.1697 | −6.801 | 0.696 | −7.679 | 1.573 |
| 3 | hr | Cmpd A 5 mg - Placebo | −2.167 | 1.0803 | 9.00 | −2.0057 | 0.0759 | −4.147 | −0.186 | −4.610 | 0.277 |
| | | Cmpd A 11.2 mg - Placebo | −2.500 | 1.3231 | 9.00 | −1.8896 | 0.0914 | −4.925 | −0.075 | −5.493 | 0.493 |
| | | Cmpd A 44.8 mg - Placebo | −1.553 | 1.5477 | 9.00 | −1.0032 | 0.3420 | −4.390 | 1.284 | −5.054 | 1.949 |
| 4 | hr | Cmpd A 5 mg - Placebo | −4.167 | 1.2544 | 9.00 | −3.3217 | 0.0089 | −6.466 | −1.867 | −7.004 | −1.329 |
| | | Cmpd A 11.2 mg - Placebo | −5.000 | 1.5363 | 9.00 | −3.2546 | 0.0099 | −7.816 | −2.184 | −8.475 | −1.525 |
| | | Cmpd A 44.8 mg - Placebo | −2.184 | 1.7971 | 9.00 | −1.2154 | 0.2551 | −5.479 | 1.110 | −6.250 | 1.881 |
| 5 | hr | Cmpd A 5 mg - Placebo | −3.500 | 1.3153 | 9.00 | −2.6610 | 0.0260 | −5.911 | −1.089 | −6.475 | −0.525 |
| | | Cmpd A 11.2 mg - Placebo | −4.000 | 1.6109 | 9.00 | −2.4831 | 0.0348 | −6.953 | −1.047 | −7.644 | −0.356 |
| | | Cmpd A 44.8 mg - Placebo | −2.026 | 1.8844 | 9.00 | −1.0753 | 0.3102 | −5.481 | 1.428 | −6.289 | 2.237 |
| 6 | hr | Cmpd A 5 mg - Placebo | −4.333 | 1.6471 | 9.00 | −2.6310 | 0.0273 | −7.353 | −1.314 | −8.059 | −0.607 |
| | | Cmpd A 11.2 mg - Placebo | −4.000 | 2.0172 | 9.00 | −1.9829 | 0.0787 | −7.698 | −0.302 | −8.563 | 0.563 |
| | | Cmpd A 44.8 mg - Placebo | −1.158 | 2.3597 | 9.00 | −0.4907 | 0.6354 | −5.484 | 3.168 | −6.496 | 4.180 |
| 7 | hr | Cmpd A 5 mg - Placebo | −4.333 | 1.8581 | 9.00 | −2.3321 | 0.0446 | −7.739 | −0.927 | −8.537 | −0.130 |
| | | Cmpd A 11.2 mg - Placebo | −4.500 | 2.2757 | 9.00 | −1.9774 | 0.0794 | −8.672 | −0.328 | −9.648 | 0.648 |
| | | Cmpd A 44.8 mg - Placebo | −3.737 | 2.6621 | 9.00 | −1.4037 | 0.1940 | −8.617 | 1.143 | −9.759 | 2.285 |
| 8 | hr | Cmpd A 5 mg - Placebo | −3.500 | 1.1204 | 9.00 | −3.1238 | 0.0122 | −5.554 | −1.446 | −6.035 | −0.965 |
| | | Cmpd A 11.2 mg - Placebo | −3.500 | 1.3722 | 9.00 | −2.5506 | 0.0312 | −6.015 | −0.985 | −6.604 | −0.396 |
| | | Cmpd A 44.8 mg - Placebo | −2.921 | 1.6052 | 9.00 | −1.8197 | 0.1022 | −5.864 | 0.022 | −6.552 | 0.710 |
| 9 | hr | Cmpd A 5 mg - Placebo | −1.500 | 1.0140 | 9.00 | −1.4792 | 0.1732 | −3.359 | 0.359 | −3.794 | 0.794 |
| | | Cmpd A 11.2 mg - Placebo | −3.000 | 1.2419 | 9.00 | −2.4156 | 0.0389 | −5.277 | −0.723 | −5.809 | −0.191 |
| | | Cmpd A 44.8 mg - Placebo | −2.868 | 1.4528 | 9.00 | −1.9744 | 0.0798 | −5.532 | −0.205 | −6.155 | 0.418 |
| 10 | hr | Cmpd A 5 mg - Placebo | −2.000 | 1.2731 | 9.00 | −1.5709 | 0.1506 | −4.334 | 0.334 | −4.880 | 0.880 |
| | | Cmpd A 11.2 mg - Placebo | −2.500 | 1.5593 | 9.00 | −1.6033 | 0.1433 | −5.358 | 0.358 | −6.027 | 1.027 |
| | | Cmpd A 44.8 mg - Placebo | −0.632 | 1.8240 | 9.00 | −0.3463 | 0.7371 | −3.975 | 2.712 | −4.758 | 3.495 |
| 11 | hr | Cmpd A 5 mg - Placebo | −0.167 | 0.9502 | 9.00 | −0.1754 | 0.8646 | −1.908 | 1.575 | −2.316 | 1.983 |
| | | Cmpd A 11.2 mg - Placebo | −2.000 | 1.1637 | 9.00 | −1.7186 | 0.1198 | −4.133 | 0.133 | −4.633 | 0.633 |
| | | Cmpd A 44.8 mg - Placebo | 0.974 | 1.3613 | 9.00 | 0.7152 | 0.4926 | −1.522 | 3.469 | −2.106 | 4.053 |

Note:
The analysis was performed using the GLM procedure because asymptotic variance matrices of covariance parameter estimates produced by the MIXED procedure were singular in some of the ANOVA models.

What is claimed is:

1. A method for treating narcolepsy type 1 in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

2. The method of claim 1, wherein Cmax for administration of Compound (I) is about 8.30 ng/mL or more.

3. The method of claim 1, wherein AUC∞ for administration of Compound (I) is about 75.6 ng*h/mL or more.

4. The method of claim 1, wherein Cmax/Dose for administration of Compound (I) is about 1.66 ng/mL/mg or more.

5. The method of claim 4, wherein the administration is non-oral administration.

6. The method of claim 1, wherein AUC∞/Dose for administration of Compound (I) is about 15.1 ng*h/mL/mg or more.

7. The method of claim 6, wherein the administration is non-oral administration.

8. The method of claim 1, wherein the administration is non-oral administration.

9. The method of claim 8, wherein the non-oral administration is intravenous administration, subcutaneous administration, transdermal administration or transmucosal administration.

10. The method of claim 9, wherein non-oral administration is intravenous administration.

11. The method of claim 1, wherein the administration is a single daily administration or a multiple daily administration.

12. A method for increasing wakefulness or decreasing excessive sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 5.04 ng/mL or more for about 1 hour or more.

13. The method of claim 12, wherein the administration is non-oral administration.

14. A method for decreasing cataplexy-like events in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

15. The method of claim 14, wherein the administration is non-oral administration.

16. A method for increasing intracellular calcium concentration in a subject in need thereof, comprising administering to the subject methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 5.04 ng/mL or more for about 1 hour or more.

17. A method for increasing sleep latency in maintenance of wakefulness test (MWT) in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 5.04 ng/mL or more for about 1 hour or more.

18. The method of claim 17, wherein the administration is non-oral administration.

19. A method for improving Karolinska Sleepiness Scale (KSS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 5.04 ng/mL or more for about 1 hour or more.

20. The method of claim 19, wherein the administration is non-oral administration.

21. A method for treating a disease associated with reduced orexin level in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 5.04 ng/mL or more for about 1 hour or more.

22. The method of claim 21, wherein the administration is non-oral administration.

23. A method for increasing alertness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 5.04 ng/mL or more for about 1 hour or more.

24. The method of claim 23, wherein the subject is suffering from or diagnosed as narcolepsy type 1.

25. A method for improving Epworth Sleepiness Scale (ESS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 5.04 ng/mL or more for about 1 hour or more.

26. The method of claim 25, wherein the subject is suffering from or diagnosed as narcolepsy type 1.

27. The method of any of claims 1-26, wherein the effective amount is between about 3 mg to about 500 mg.

28. The method of claim 27, wherein the effective amount is between about 5 mg to about 300 mg.

29. The method of claim 27, wherein the effective amount is between about 5 mg to about 100 mg.

30. The method of claim 27, wherein the effective amount is between about 5 mg to about 50 mg.

31. The method of any of claims 1-26, wherein Compound (I) is administered at least once per day.

32. The method of any of claims 1-26, further comprising administering one or more additional therapies.

33. The method of claim 32, wherein the one or more additional therapies is selected from a stimulant, antidepressant, central nervous system depressant, and histamine 3 (H3) receptor antagonist.

34. A pharmaceutical composition comprising (a) methyl 3-((methylsulfonyl)amino)-2-(((4-phenylcyclohexyl)oxy)methyl)piperidine-1-carboxylate (Compound (I)), or a salt thereof, and (b) a pharmaceutically acceptable carrier therefor,
which provides a plasma concentration for Compound (I) of about 5.04 ng/mL or more for about 1 hour or more.

35. The pharmaceutical composition of claim 34, which provides a Cmax for Compound (I) of about 8.30 ng/mL or more.

36. The pharmaceutical composition of claim 34 or 35, which provides an AUC∞ for Compound (I) of about 75.6 ng*h/mL or more.

37. The pharmaceutical composition of claim 34, 35 or 36, which is formulated for non-oral administration.

38. The method of any of claims 1-33, wherein Compound (I) is an optically active compound.

39. The method of any of claims 1-33, wherein Compound (I) is methyl (2R,3S)-3-((methylsulfonyl)amino)-2-(((cis-4-phenylcyclohexyl)oxy)methyl)piperidine-1-carboxylate (Compound (A)).

40. The method of any of claims 1-33, 38, and 39, wherein the plasma concentration for Compound (I) of about 5.04 ng/ml or more for a period of about 1 hour or more represents an average plasma concentration level for a group of treated subjects and the time period of 1 hour or more begins at any time point following administration.

41. The method of any of claims 1-33, 38 and 39, wherein the plasma concentration for Compound (I) of about 5.04 ng/ml or more for a period of about 1 hour or more represents the plasma concentration level for the individually treated subject and the time period of 1 hour or more begins at any time point following administration to that subject.
